(19) United States
(12) Patent Application Publication
(10) Pub. No.: US 2026/0151417 A1
(43) Pub. Date: Jun. 4, 2026

(54) Nucleic acid regulation of c9orf72

(71) Applicant: uniQure biopharma B.V., Amsterdam (NL)

(72) Inventors: Vanessa Zancanella, Amsterdam (NL); Ying Pui Liu, Amsterdam (NL); Irena Bockaj, Amsterdam (NL); Morgane Sarah Wartel, Amsterdam (NL); Sebastian Niklas Kieper, Amsterdam (NL); Astrid Valles Sánchez, Amsterdam (NL); Seyda Acar Broekmans, Amsterdam (NL); Melvin Maurice Evers, Amsterdam (NL)

(73) Assignee: uniQure biopharma B.V., Amsterdam (NL)

(21) Appl. No.: 18/851,092

(22) PCT Filed: Apr. 11, 2023
(86) PCT No.: PCT/EP2023/059431

§ 371 Date: Sep. 26, 2024

(30) Foreign Application Priority Data
Apr. 12, 2022 (EP) 22167884.0
Oct. 7, 2022 (EP) 22200265.1

(51) Int. Cl. A61K 31/7088; A61K 45/06; A61K 48/00; A61P 25/28; C12N 15/113; C12N 15/864

(52) U.S. Cl. CPC A61K 31/7088; A61K 45/06; A61K 48/005; A61P 25/28; C12N 15/1137; C12N 15/8645; C12N 2310/531; C12N 2750/14143; C12N 2830/34

(57) ABSTRACT

The present invention relates to a nucleic acid comprising two or more RNA encoding sequences, wherein at least one of the sequences comprises a guide sequence substantially complementary to part of the C9orf72 gene. The invention also relates to associated AAVs, compositions, pharmaceutical compositions and uses in treatments thereof.

### FIELD OF THE INVENTION

The present invention relates to the fields of biotechnology, medicine and gene therapy. Specifically, the invention relates to a nucleic acid comprising two or more RNA encoding sequences, wherein at least one of the sequences comprises a guide sequence substantially complementary to part of the c9orf72 gene. The invention also relates to associated AAVs, compositions, pharmaceutical compositions and uses in treatments thereof.

### BACKGROUND OF THE INVENTION

Amyotrophic lateral sclerosis (ALS) is a uniformly fatal disease characterized by degeneration of upper and lower motor neurons, leading to progressive paralysis, respiratory failure, and death in 2-5 years after diagnosis. It is also the most common adult motor neuron disease, with a prevalence of 5 per 100,000 and a lifetime risk of 1:400-1:800. About a third of those with ALS also develop frontotemporal dementia (FTD), a destruction of neurons in the brain that causes profound personality changes and disability. The two diseases are similar in both pathology and genetics.

One of the therapeutical approaches for ALS is targeting genetic mutations known to cause ALS. Among these, expansion of a GGGGCC (G4C2) hexanucleotide repeat in the human chromosome 9 open reading frame 72 (c9orf72) gene has been found in 40% of familial and 8% of sporadic cases of ALS.

The c9orf72 locus is located on the reverse strand of chromosome 9 (41 kb). It includes two non-coding exons (1a and 1b) and 10 coding exons (from 2 to 11); the G4C2 repeat is localized between the two non-coding exons (1a and 1b). c9orf72 gives rise to three coding variants. Variant 1, V1 (NM_145005), is a short transcript including non-coding exon 1a and exons 2 to 5 as the coding sequence. V2 (NM_018325) and V3 (NM_001256054) differ in their inclusion of the non-coding exon 1b or 1a, respectively, and share exons 2 to 11 as the coding sequence. Alternative splicing of these three RNA variants results in the production of two different isoforms: the 222-amino acid (aa) isoform (C9-short of 24 kDa) encoded by V1, while the 481-aa isoform (C9-long of 54 kDa) is encoded by V2 and V3. In most people, the repeat length is 2. However, in a pathological state, the G4C2 repeat is abnormally expanded. Hundreds to thousands of repeats may be observed in ALS patients, with a small proportion of patients having an intermediate expansion of the order of 20 to 30 repeats in size. It has been suggested that a threshold of 20 or 23 repeats may be used to discriminate between pathogenic and neutral expansions, with an expansion of 24 or more repeats is considered pathogenic (lacoangeli, A. et al. 2019 Acta Neuropathol Commun 7, 115).

Bidirectional transcription of the hexanucleotide repeat expansion (HRE) generates G4C2 sense and G2C4 antisense expanded RNAs. The presence of G4C2 expanded transcripts is associated to two main pathological mechanisms: a loss-of-function effect, causing c9orf72 haploinsufficiency, and a gain of function associated with the expression of abnormal bidirectionally transcribed RNAs carrying the repeat that accumulate in RNA foci or are translated into dipeptide repeat proteins (DPRs) via repeat-associated non-ATG (RAN) translation.

Previous attempts to target these repeat expansions include single-stranded silencing RNAs (ss-siRNAs) (Hu J. et al. 2015Chem. Biol. 22:1505-1511) and engineered artificial anti-c9orf72-targeting miRNAs (miC) (Martier R et al. 2019 Mol Ther Nucleic Acids. 14:593-608). The silencing efficacy of adeno-associated virus (AAV) 5-miC has been tested in human-derived induced pluripotent stem cell (iPSC) neurons and in an ALS mouse model (Martier R et al. 2019 Mol Ther Nucleic Acids. 16:26-37).

These attempts based on ss-siRNAs and/or engineered artificial anti-c9orf72-targeting miRNAs indistinctively targeted all three coding variants of the c9orf72 gene. Additionally, there may be potential toxicity issues related to the undesired expression of the passenger strands.

There is, therefore, still a need to develop new gene therapy strategies to effectively target pathological variants of the c9orf72 gene and to minimize potential toxicity issues.

### SUMMARY OF THE INVENTION

The present invention solves the problem in the prior art by using nucleic acids comprising at least two RNA encoding sequences, wherein each of the RNA comprises a hairpin and at least one of the RNA encoding sequences comprises a guide sequence substantially complementary to part of a c9orf72 gene of choice, preferably the human c9orf72 (NCBI accession number: NG_031977.2). The invention provides for a highly versatile system, which allows for the simultaneous use of different guide sequences. As shown herein, the invention provides for an optimized and highly potent inhibition of RNA expression. Consequently, when using the nucleic acids or AAVs of the invention in gene therapy, a significant decrease not only in the concentration of each dose but also in the number of doses needed (single dose administration) is to be expected. This in turn results in lower toxicity and immunogenicity risks associated with the use of said nucleic acids or AVVs of the invention. Additionally, the costs of the therapy based on the nucleic acids or AAVs of the invention are expected to be lower than other gene therapy products, presenting a substantial economic advantage.

Thus, a first aspect of the invention relates to a nucleic acid comprising a sequence encoding a first RNA and a sequence encoding a second RNA (“nucleic acid of the invention”), wherein: the first and second RNA each comprise a hairpin; the second RNA comprises a guide sequence of at least 19 nucleotides substantially complementary to part of the c9orf72 gene; and the genomic sequence of the c9orf72 gene comprises a G4C2 hexanucleotide repeat expansion of at least 24 repeats in a non-coding region.

A second aspect of the invention relates to an expression cassette comprising the nucleic acid of the invention, where the expression cassette is a DNA molecule.

A third aspect of the invention relates to an adeno-associated virus (AAV) vector (“AAV vector of the invention”) comprising the nucleic acid or expression cassette of the invention.

A fourth aspect of the invention relates to pharmaceutical compositions comprising the nucleic acid, expression cassette, or the AAV vector of the invention.

A fifth aspect of the invention relates to the use as a medicament of the nucleic acid, the expression cassette, the AAV vector or the pharmaceutical composition of the invention.

A sixth aspect of the invention relate to a kit comprising the nucleic acid, the expression cassette, the AAV vector, or pharmaceutical composition of the invention.

A seventh aspect of the invention relates to cells comprising the nucleic acid, the cassette or the AAV vector of the invention.

Further aspects of the invention relate to a kit comprising the nucleic acid, AAV vector, or pharmaceutical composition of the invention, and to cells comprising the nucleic acid or the AAV vector of the invention.

### DESCRIPTION OF THE INVENTION

### Definitions

Unless defined otherwise, technical and scientific terms used herein have the same meaning as commonly understood by one of ordinary skill in the art to which this disclosure belongs. One skilled in the art will recognize many methods and materials similar or equivalent to those described herein, which could be used in the practice of the present invention.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

For the purposes of the present invention, the term “obtained” is considered to be a preferred embodiment of the term “obtainable”. If hereinafter e.g. an antibody is defined to be obtainable from a specific source, this is also to be understood to disclose an antibody which is obtained from this source.

As used herein, the term “and/or” indicates that one or more of the stated cases may occur, alone or in combination with at least one of the stated cases, up to with all of the stated cases.

As used herein, with “At least” a particular value means that particular value or more. For example, “at least 2” is understood to be the same as “2 or more” i.e., 2, 3, 4, 5, 6, 7, 8, 9, 10, 11, 12, 13, 14, 15, . . . ,etc.

The word “about” or “approximately” when used in association with a numerical value (e.g. about 10) preferably means that the value may be the given value (of 10) more or less 0.1% of the value.

As used herein, “an effective amount” is meant the amount of an agent required to ameliorate the symptoms of a disease relative to an untreated patient. The effective amount of active agent(s) used to practice the present invention for therapeutic treatment of, for example a cancer, varies depending upon the manner of administration, the age, body weight, and general health of the subject. Ultimately, the attending physician or veterinarian will decide the appropriate amount and dosage regimen. Such amount is referred to as an “effective” amount, which may (in the case of a viral delivery vector) be determined as genome copies per kilogram (GC/kg). Thus, in connection with the administration of a drug which, in the context of the current disclosure, is “effective against” a disease or condition indicates that administration in a clinically appropriate manner results in a beneficial effect for at least a statistically significant fraction of patients, such as an improvement of symptoms, a cure, a reduction in at least one disease sign or symptom, extension of life, improvement in quality of life, or other effect generally recognized as positive by medical doctors familiar with treating the particular type of disease or condition.

The use of a substance as a medicament as described in this document can also be interpreted as the use of said substance in the manufacture of a medicament. Similarly, whenever a substance is used for treatment or as a medicament, it can also be used for the manufacture of a medicament for treatment. Products for use as a medicament described herein can be used in methods of treatments, wherein such methods of treatment comprise the administration of the product for use.

The terms “homology”, “sequence identity” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods.

“Sequence identity” and “sequence similarity” can be determined by alignment of two peptide or two nucleotide sequences using global or local alignment algorithms, depending on the length of the two sequences. Sequences of similar lengths are preferably aligned using global alignment algorithms (e.g. Needleman Wunsch) which align the sequences optimally over the entire length, while sequences of substantially different lengths are preferably aligned using local alignment algorithms (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have a substantially different overall length, local alignments, such as those using the Smith Waterman algorithm, are preferred.

Alternatively, percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. 1990 J. Mol. Biol. 215:403-410. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., 1997 Nucleic Acids Res. 25 (17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

Within the context of the present invention, the term “variant thereof”, when referring to a given sequence (SEQ ID), includes any nucleic acids which retain at least some of the properties of the corresponding native nucleic, for example, reduction of RNA expression. The term “variant” may include any nucleic acids with at least 50; 55; 60; 65; 70; 75; 80; 90; or 95% sequence identity with the native nucleic acid.

In some embodiments, the sequence of the nucleic acid of the invention is codon optimised. Codon optimisation refers to experimental approaches designed to improve the codon composition of a recombinant gene based on various criteria without altering the amino acid sequence. This is possible because most amino acids are encoded by more than one codon. Most codon-optimization approaches avoid the use of rare codons. However, different approaches vary in the extent of other features considered, including mRNA elements that can inhibit expression, nucleotide context of the initiation codon, mRNA secondary structures, sequence repeats, nucleotide composition, internal ribosome entry sites, promoter sequences, and putative splice donor and acceptor sites. In addition, some programs consider protein structural information, intragenic poly (A) sites, stop codons in alternative reading frames, and dinucleotides that are targets for RNase cleavage, mutation, and methylation-dependent gene silencing. The person skilled in the art has within their understanding the requirements needed to design such a codon-optimised nucleic acid.

As used herein, the term “selectively hybridizing”, “hybridizes selectively” and similar terms are intended to describe conditions for hybridization and washing under which nucleotide sequences at least 66%, at least 70%, at least 75%, at least 80%, more preferably at least 85%, even more preferably at least 90%, preferably at least 95%, more preferably at least 98% or more preferably at least 99% homologous to each other typically remain hybridized to each other. That is to say, such hybridizing sequences may share at least 45%, at least 50%, at least 55%, at least 60%, at least 65, at least 70%, at least 75%, at least 80%, more preferably at least 85%, even more preferably at least 90%, more preferably at least 95%, more preferably at least 98% or more preferably at least 99% sequence identity.

A preferred, non-limiting example of such hybridization conditions is hybridization in 6X sodium chloride/sodium citrate (SSC) at about 45° C., followed by one or more washes in 1×SSC, 0.1% SDS at about 50° C., preferably at about 55° C., preferably at about 60° C. and even more preferably at about 65° C.

Highly stringent conditions include, for example, hybridization at about 68° C. in 5×SSC/5×Denhardt's solution/1.0% SDS and washing in 0.2×SSC/0.1% SDS at room temperature. Alternatively, washing may be performed at 42° C.

The skilled artisan will know which conditions to apply for stringent and highly stringent hybridization conditions. Additional guidance regarding such conditions is readily available in the art, for example, in Sambrook et al., 1989 Molecular Cloning, A Laboratory Manual, Cold Spring Harbor Press, N.Y.;

and Ausubel et al. (eds.), Sambrook and Russell 2001 “Molecular Cloning: A Laboratory Manual (3^{rd} edition), Cold Spring Harbor Laboratory, Cold Spring Harbor Laboratory Press, New York 1995, Current Protocols in Molecular Biology, (John Wiley & Sons, N.Y.).

Of course, a polynucleotide which hybridizes only to a poly A sequence (such as the 3′ terminal poly (A) tract of mRNAs), or to a complementary stretch of T (or U) residues, would not be included in a polynucleotide of the invention used to specifically hybridize to a portion of a nucleic acid of the invention, since such a polynucleotide would hybridize to any nucleic acid molecule containing a poly (A) stretch or the complement thereof (e.g., practically any double-stranded cDNA clone).

A “nucleic acid construct” or “nucleic acid vector” is herein understood to mean a man-made nucleic acid molecule resulting from the use of recombinant DNA technology. The term “nucleic acid construct” therefore does not include naturally occurring nucleic acid molecules although a nucleic acid construct may comprise (parts of) naturally occurring nucleic acid molecules. A “vector” is a nucleic acid construct (typically DNA or RNA) that serves to transfer an exogenous nucleic acid sequence (i.e. DNA or RNA) into a host cell. A vector is preferably maintained in the host by at least one of autonomous replication and integration into the host cell's genome. The terms “expression vector” or “expression construct” refer to nucleotide sequences that are capable of affecting expression of a gene in host cells or host organisms compatible with such sequences. These expression vectors typically include at least one “expression cassette” that is the functional unit capable of affecting expression of a sequence encoding a product to be expressed and wherein the coding sequence is operably linked to the appropriate expression control sequences, which at least comprises a suitable transcription regulatory sequence and optionally, 3′ transcription termination signals. Additional factors necessary or helpful in affecting expression may also be present, such as expression enhancer elements. The expression vector will be introduced into a suitable host cell and be able to affect expression of the coding sequence in an in vitro cell culture of the host cell. A preferred expression vector will be suitable for expression of viral proteins and/or nucleic acids, particularly recombinant AAV proteins and/or nucleic acids.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer or biological entity.

The term “reporter” may be used interchangeably with marker, although it is mainly used to refer to visible markers, such as green fluorescent protein (GFP) or luciferase.

The terms “protein” or “polypeptide” are used interchangeably and refer to molecules consisting of a chain of amino acids, without reference to a specific mode of action, size, 3-dimensional structure or origin.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′-untranslated sequence (3′-end) comprising a polyadenylation site. “Expression of a gene” refers to the process wherein a DNA region which is operably linked to appropriate regulatory regions, particularly a promoter, is transcribed into an RNA, which is biologically active, i.e. which is capable of being translated into a biologically active protein or peptide.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. In this context, the use of only “homologous” sequence elements allows the construction of “self-cloned” genetically modified organisms (GMO's) (self-cloning is defined herein as in European Directive 98/81/EC Annex II). When used to indicate the relatedness of two nucleic acid sequences the term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed later.

The terms “heterologous” and “exogenous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous and exogenous nucleic acids or proteins are not endogenous to the cell into which they are introduced but have been obtained from another cell or are synthetically or recombinantly produced. Generally, though not necessarily, such nucleic acids encode proteins, i.e. exogenous proteins, that are not normally produced by the cell in which the DNA is transcribed or expressed. Similarly, exogenous RNA encodes for proteins not normally expressed in the cell in which the exogenous RNA is present. Heterologous/exogenous nucleic acids and proteins may also be referred to as foreign nucleic acids or proteins. Any nucleic acid or protein that one of skill in the art would recognize as foreign to the cell in which it is expressed is herein encompassed by the term heterologous or exogenous nucleic acid or protein. The terms heterologous and exogenous also apply to non-natural combinations of nucleic acid or amino acid sequences, i.e. combinations where at least two of the combined sequences are foreign with respect to each other.

As used herein, the term “non-naturally occurring” when used in reference to an organism means that the organism has at least one genetic alternation that is not normally found in a naturally occurring strain of the referenced species, including wild-type strains of the referenced species. Genetic alterations include, for example, modifications introducing expressible nucleic acids encoding proteins or enzymes, other nucleic acid additions, nucleic acid deletions, nucleic acid substitutions, or other functional disruption of the organism's genetic material. Such modifications include, for example, coding regions and functional fragments thereof for heterologous or homologous polypeptides for the referenced species. Additional modifications include, for example, non-coding regulatory regions in which the modifications alter expression of a gene or operon. Genetic modifications to nucleic acid molecules encoding enzymes, or functional fragments thereof, can confer a biochemical reaction capability or a metabolic pathway capability to the non-naturally occurring organism that is altered from its naturally occurring state.

As used herein, the term “operably linked” refers to a linkage of polynucleotide (or polypeptide) elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

An expression control sequence is “operably linked” to a nucleotide sequence when the expression control sequence controls and regulates the transcription and/or the translation of the nucleotide sequence. Thus, an expression control sequence can include promoters, enhancers, internal ribosome entry sites (IRES), transcription terminators, a start codon in front of a protein-encoding gene, splicing signal for introns, and stop codons.

The term “expression control sequence” is intended to include, at a minimum, a sequence whose presence is designed to influence expression, and can also include additional advantageous components. For example, leader sequences and fusion partner sequences are expression control sequences. The term can also include the design of the nucleic acid sequence such that undesirable, potential initiation codons in and out of frame, are removed from the sequence. It can also include the design of the nucleic acid sequence such that undesirable potential splice sites are removed. It includes sequences or polyadenylation sequences (pA) which direct the addition of a polyA signal, i.e., a string of adenine residues at the 3′-end of a mRNA, sequences referred to as polyA sequences. It also can be designed to enhance mRNA stability. Expression control sequences which affect the transcription and translation stability, e.g., promoters, as well as sequences which affect the translation, e.g., Kozak sequences, are known in insect cells.

Expression control sequences can be of such nature as to modulate the nucleotide sequence to which it is operably linked such that lower expression levels or higher expression levels are achieved.

### DETAILED DESCRIPTION OF THE INVENTION

A first aspect of the invention relates to a nucleic acid comprising a sequence encoding a first RNA and a sequence encoding a second RNA, wherein the first and second RNA each comprise a hairpin; the second RNA comprises a guide sequence of at least 19 nucleotides substantially complementary to part of the c9orf72 gene; and wherein the c9orf72 gene comprises a GGGGCC (G4C2) hexanucleotide repeat expansion of at least 24 repeats in a non-coding region.

### Nucleic Acids

The term “nucleic acid” as used herein takes its regular meaning in the art. Thus, the term “RNA” or “RNA molecule” or “ribonucleic acid molecule” as used herein refers to a polymer of ribonucleotides (e.g., 2, 3, 4, 5, 10, 15, 20, 25, 30, or more ribonucleotides) and the term “DNA” or “DNA molecule” or “deoxyribonucleic acid molecule” as used herein refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssDNA and ssRNA, respectively) or multi-stranded (e.g., double stranded, i.e., dsDNA and dsRNA, respectively). “mRNA” or “messenger RNA” is single-stranded RNA that specifies the amino acid sequence of one or more polypeptide chains. This information is translated during protein synthesis when ribosomes bind to the mRNA.

As used herein, the term “small interfering RNA” (“siRNA”) (also referred to in the art as “short interfering RNAs”) refers to an RNA duplex (or RNA analog) comprising about 10-50 nucleotides (or nucleotide analogs) which is capable of directing or mediating RNA interference. Preferably, an siRNA comprises between about 15-30 nucleotides or nucleotide analogs, more preferably between about 16-25 nucleotides (or nucleotide analogs), even more preferably between about 18-23 nucleotides (or nucleotide analogs), and even more preferably between about 19-22 nucleotides (or nucleotide analogs) (e.g., 19, 20, 21 or 22 nucleotides or nucleotide analogs). The term “short” siRNA refers to an siRNA comprising about 21 nucleotides (or nucleotide analogs), for example, 19, 20, 21 or 22 nucleotides. The term “long” siRNA refers to an siRNA comprising about 24-25 nucleotides, for example, 23, 24, 25 or 26 nucleotides. Short siRNAs may, in some instances, include fewer than 19 nucleotides, e.g., 16, 17 or 18 nucleotides, provided that the shorter siRNA retains the ability to mediate RNAi. Likewise, long siRNAs may, in some instances, include more than 26 nucleotides, provided that the longer siRNA retains the ability to mediate RNAi absent further processing, e.g., enzymatic processing, to a short siRNA.

As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be induced, for example, to silence the expression of target genes. Double stranded RNA structures that are suitable for inducing RNAi are well known in the art. For example, a small interfering RNA (siRNA) can induce RNAi. An siRNA comprises two separate RNA strands, one strand comprising a first RNA sequence and the other strand comprising a second RNA sequence, thus a first and a second strand. An siRNA design that is often used involves consecutive base pairs with a 3′ overhang. The first and/or second strand may comprise a 3′-overhang. The 3′-overhang preferably is a dinucleotide overhang on both strands of the siRNA. Such a design is based on observed Dicer processing of larger double stranded RNAs that results in siRNAs having these features. The 3′-overhang may be comprised in the first strand. The 3′-overhang may be in addition to the first strand. The length of the two strands of which an siRNA is composed may be 19, 20, 21, 25 22, 23, 24, 25, 26 or 27 nucleotides or more.

siRNAs may also serve as Dicer substrates. For example, a Dicer substrate may be a 27-mer consisting of two strands of RNA that have 27 consecutive base pairs. The first strand is positioned at the 3′-end of the 27-mer duplex. At the 3′-ends, like with siRNAs, each or one of the strands may comprise a two nucleotide overhang. The 3′-overhang may be comprised in the first strand. The 3′-overhang may be in addition to the first strand. 5′ from the first strand, additional sequences may be included that are either complementary to the target RNA sequence adjacent or not. The other end of the siRNA Dicer substrate is blunt ended. This Dicer substrate design may result in a preference in processing by Dicer such that an siRNA can be formed like the siRNA design as described above, having 19 consecutive base pairs and 2 nucleotide overhangs at both 3′-ends.

In any case, siRNAs, or the like, are composed of two separate RNA strands (Fire et al. 1998 Nature. 1998 19; 391 (6669): 806-1 1), each RNA strand comprising or consisting of the first and second RNA strand of the two or more RNAs in accordance with the invention. Thus, the nucleic acid of the invention may be said to derive a first and second RNA strand, which is the first RNA, and a third and fourth RNA strand, which is the second RNA. In some embodiments, the nucleic acid of the invention may be said to derive a first and second RNA strand, which is the first RNA, a third and fourth RNA strand, which is the second RNA, and, where applicable, a fifth and a sixth strand, which is the third RNA. Alternative naming conventions for each first, second, third, fourth, fifth and sixth RNA strands are within the scope of the invention, which may be complementary, substantially complementary, or unique to each other, or in any other required arrangement as discussed herein.

The loop sequence may also be a stem-loop sequence, whereby the double stranded region of the shRNA is extended. Like the siRNA Dicer substrate described above, a shRNA can be processed by e.g. Dicer to provide for an siRNA having an siRNA design such as described above, having e.g. 19 consecutive base pairs and 2 nucleotide overhangs at both 3′-ends. In case the shRNA is to be processed by Dicer, it is preferred to have the first and second strands at the end of the shRNA, i.e. such that the putative strands of the siRNA are linked via a stem loop sequence: 5′-first strand-stem loop sequence-second strand-optional 2 nt overhang sequence-3′. Or, conversely, 5′-second strand-stem loop sequence-first strand-optional 2 nt overhang sequence-3′. Another shRNA design may be a shRNA structure that is processed by the RNAi machinery to provide for an activated RNA-induced silencing complex (RISC) that does not require Dicer processing (Liu et al. 2013 Nucleic Acids Res. 41 (6): 3723-33, incorporated herein by reference), so called AgoshRNAs, which are based on a structure very similar to the miR-451 scaffold as described below. Such a shRNA structure comprises in its loop sequence part of the first RNA sequence. Such an shRNA structure may also consist of the first strand, followed immediately by the second strand.

### Spacers

In some embodiments of the nucleic acid of the invention, the sequence encoding the first RNA is followed by a spacer comprising at least 15 nucleotides (also referred to as nt or nts through this application) followed by the sequence encoding the second RNA. Thus, preferably, in a 5′ to 3′ direction, the sequence encoding the first RNA is followed by a first spacer comprising at least 15 nucleotides, which spacer is followed by the sequence encoding the second RNA. It is understood that the 5′ to 3′ direction refers to the coding strand in case of a double-stranded (ds) nucleic acid.

As detailed above, the nucleic acid may be said to derive a first and second RNA strand, which is the first RNA, and a third and fourth RNA strand, which is the second RNA, wherein the sequences encoding the first and second RNA strands are followed by a spacer of at least 15 nucleotides followed by the sequences encoding the third and fourth RNA strands.

In some embodiments, the nucleic acid of the invention comprises a third sequence encoding a third RNA.

In preferred embodiments of the invention, in a 5′ to 3′ direction, the sequence encoding the second RNA is followed by a second spacer comprising at least 15 nucleotides, which second spacer is followed by the sequence encoding the third RNA.

Thus, the nucleic acid may be said to derive a first and second RNA strand, which is the first RNA, a third and fourth RNA strand, which is the second RNA, and a fifth and sixth RNA strands, which is the third RNA, wherein the sequences encoding the first and second RNA strands are followed by a spacer of at least 15 nucleotides followed by the sequences encoding the third and fourth RNA strands, said third and fourth strands followed by a spacer of at least 15 nucleotides and the sequences encoding the fifth and sixth RNA strands.

In some embodiments of the invention, the spacer comprises at least 25; at least 30; or at least 35 nucleotides.

In some embodiments of the invention, the spacer comprises at least: 15; 20; 25; 30; 35; 40; 45; 50; 55; 60; 65; 70; 75; 80; 85; 90; 95; 100; 105; 110; 115; 120; 125; 130; 135; 140; 145; 150; 155; 160; 165; 170; 175; 180; 185; 190; 195; or 200 nucleotides.

In some specific embodiments, the spacer of the invention comprises 15; 16; 17; 18; 19; 20; 21; 22; 23; 24; 25; 26; 27; 28; 29; 30; 31; 32; 33; 34; 35; 36; 37; 38; or 40 nucleotides.

In some specific embodiments of the invention, the spacer comprises 75; 76; 77; 78; 79; 80; 81; 82; 83; 84; 85; 86; 87; 88; 89; 90; 91; 92; 93; 94; 95; 96; 97; 98; 99; 100; 101; 102; 103; 104; or 105 nucleotides.

In some embodiments of the invention, the spacer comprises or consists of a sequence selected from the group consisting of: SEQ ID NO. 225; SEQ ID NO. 226; SEQ ID NO. 227; and variants thereof.

Thus, in some specific embodiments of the invention, the sequences encoding the first and second RNA strands are followed by a spacer of at least 75 nucleotides and the sequences encoding the third and fourth RNA strands.

In some embodiments of the invention, the sequences encoding the first and second RNA strands are followed by a spacer comprising or consisting of SEQ ID NO. 225 and the sequences encoding the third and fourth RNA strands.

In some specific embodiments, the sequences encoding the first and second RNA strands are followed by a spacer of at least 75 nucleotides and the sequences encoding the third and fourth

RNA strands are followed by a spacer of at least 15 nucleotides and the sequences encoding the fifth and sixth RNA strands. In some specific examples of the invention, the sequences encoding the first and second RNA strands are followed by a spacer comprising or consisting of SEQ ID NO. 225 and the sequences encoding the third and fourth RNA strands are followed by a spacer comprising or consisting of SEQ ID NO. 227 and the sequences encoding the fifth and sixth RNA strands.

In some embodiments, if one or both RNAs are shRNA to be processed by Dicer, the relevant shRNA structures mentioned above are also applicable. In some embodiment, if one or both RNAs are AgoshRNAs, the relevant shRNA structures mentioned above are also applicable. Thus, one or both RNAs may be processed by the same or different RNAi machinery (Dicer or Drosha, as described above).

### RNA Scaffolds

A double stranded RNA according to the invention may be incorporated in a pre-miRNA or pri-miRNA scaffold. MicroRNAs, i.e. miRNA, are guide strands that originate from double stranded RNA molecules that are endogenously expressed e.g. in mammalian cells. A miRNA is processed from a pre-miRNA precursor molecule, similar to the processing of a shRNA or an extended siRNA as described above, by the RNAi machinery and incorporated in RISC (Tijsterman M, Plasterk RH. Dicers at RISC; the mechanism of RNAi. Cell. 2004 Apr. 2;1 17 (1): 1-3). A pre-miRNA is a hairpin RNA molecule that can be part of a larger RNA molecule (pri-miRNA), e.g. comprised in an intron, which is first processed by Drosha to form a pre-miRNA hairpin molecule. The pre-miRNA molecule is a shRNA-like molecule that can subsequently be processed by Dicer to result in an siRNA-like double stranded RNA duplex. The miRNA, i.e. the guide strand, that is part of the double stranded RNA duplex is subsequently incorporated in RISC.

miR-144

The first RNA of the nucleic acid of the invention may be incorporated in a pre-miRNA or a pri-miRNA scaffold derived from miR-144. In other words, and as described above, the first and second strands of the first RNA of the invention are incorporated in a pre-miRNA or a pri-miRNA scaffold derived from miR-144. Thus, the first RNA can be described as a hairpin or a double stranded RNA that is substantially complementary to itself. In some embodiments, the hairpin in the first RNA comprises at least 70 nucleotides.

In some specific embodiments, the nucleic acid of the invention comprises a first, a second and a third RNA, wherein the first RNA is incorporated in a pre-miRNA or a pri-miRNA scaffold derived from miR-144 and the second and third RNA are incorporated in a pre-miRNA or a pri-miRNA scaffold derived from miR-451. Preferably, the sequence encoding the second RNA is followed by a spacer comprising at least 15 nucleotides and the sequence encoding the third RNA.

In specific embodiments, the first RNA comprises SEQ ID NO. 181 or a variant thereof. In some embodiments, the variant thereof is SEQ ID NO. 228. In more specific embodiments, the first RNA comprises SEQ ID NO 181 or a variant thereof and the second and third RNA comprises SEQ ID NO. 229 or a variant thereof. In said embodiments, the first RNA is processed by Dicer; therefore, the putative strands of the subsequent siRNA are linked via a stem loop sequence: 5′-first strand-apical loop sequence-second strand-optional 2 nt overhang sequence-3′ or, conversely, 5′-second strand-apical loop sequence-first strand-optional 2 nt overhang sequence-3′.

Additionally, in some embodiments where the first RNA is incorporated in a pre-miRNA or a pri-miRNA scaffold derived from miR-144, the first RNA is mutated to reduce processing and/or expression of the first RNA. In some specific embodiments, SEQ ID NO. 181 or the variant thereof is mutated to reduce processing and/or expression of the third RNA.

In some embodiments, the mutation is a single point mutation. In other words, the first RNA comprises a single point mutation to reduce processing and/or expression of the first RNA. Any mismatch, bulge or GU wobble introduced within positions 4-8 of the DROSHA cleavage site may impair the enzymatic activity of DROSHA. Double and triple mismatches, bulges or wobbles within said positions further decrease the activity of DROSHA. Therefore, any of the following single nucleotide polymorphisms (SNPs) and combinations thereof within the 4-8 nucleotide stretch of mir-144 may alter (pre-)-mir-144 expression.

In some embodiments, the first RNA comprises at least one mutation selected from the group consisting of: T>G at position 4; A>T or G at position 5; T>A at position 6; C>G or T at position 7; and A>T or G at position 8.

In some preferred embodiments, the first RNA comprises a single point mutation A>T at position 5. The skilled person can easily determine whether this is the case by using standard assays and appropriate controls such as described in the examples and as known in the art.

The specific combination of miR-144/miR-451 scaffolds, specifically those comprising SEQ ID NO. 228 or SEQ ID NO. 181 and those comprising SEQ ID NO. 229, within the nucleic acid of the invention is particularly helpful within the context of gene therapy and RNA silencing. miR-144 and miR-451 are examples of clustered miRNAs regulated in trans, wherein miR-144 regulates the processing of miR-451 by Ago2. Specifically, miR-144 enhances miR-451 biogenesis in trans by repressing Dicer and, in turn, repressing global canonical miRNA processing (Kretov et al. 2020, Molecular Cell 78, 317-328). Thus, where this combination of scaffolds is used, the first RNA of the invention plays a key role in enhancing the biogenesis of the second and third RNA of the invention, and therefore, the delivery of the guide sequences comprised in said first and second RNAs.

The second RNA of the nucleic acid of the invention comprise a guide sequence of at least 19 nucleotides substantially complementary to part of the c9orf72 gene. In some embodiments of the invention, as described below, the first RNA of the nucleic acid also comprises a guide sequence of at least 19 nucleotides substantially complementary to part of the c9orf72 gene. In some specific embodiments of the invention where the nucleic acid comprises a sequence encoding a third RNA, said third RNA also comprises a guide sequence of at least 19 nucleotides substantially complementary to part of the c9orf72 gene. In preferred embodiments of the invention, the c9orf72 gene is a human gene.

An RNA molecule such as present in nature, i.e. a pri-miRNA, a pre-miRNA or a miRNA duplex, may be used as a scaffold for producing an artificial miRNA that specifically targets a gene of choice. Based on the predicted RNA structure of the RNA molecule as present in nature, e.g. as predicted using e.g. m-fold software using standard settings (Zuker. Nucleic Acids Res. 31 (13), 3406-3415, 2003), the natural miRNA sequence as it is present in the RNA structure (i.e. duplex, pre-miRNA or pri-miRNA), and the sequence present in the structure that is substantially complementary therewith are removed and replaced with a first strand and a second strand according to the invention, that are the first strand and second strand of the first RNA, or the first strand and second strand of the second RNA, which may also be referred to as the third and fourth strands. Thus, using the first and second strand for solely exemplary purpose, the first strand and the second strand are preferably selected such that the predicted secondary RNA structures that are formed, i.e. of the pre-miRNA, pri-miRNA and/or miRNA duplex, resemble the corresponding predicted original secondary structure of the natural RNA sequences. pre-miRNA, pri-miRNA and miRNA duplexes (that consist of two separate RNA strands that are hybridized via complementary base pairing) as found in nature, are often not fully base paired, i.e. not all nucleotides that correspond with the first and second strand as defined above are base paired, and the first and second strand are often not of the same length. How to use miRNA precursor molecules as scaffolds for any selected target RNA sequence and substantially complementary first strand is described e.g. in Liu YP Nucleic Acids Res. 2008 36 (9): 281 1-24.

A pri-miRNA can be processed by the RNAi machinery of the cell. The pri-miRNA comprising flanking sequences at the 5′-end and the 3′-end of a pre-miRNA hairpin and/or shRNA like molecule. Such a pri-miRNA hairpin can be processed by Drosha to produce a pre-miRNA. The length of the flanking sequences can vary but may be around 80 nt in length (Zeng and Cullen, J Biol Chem. 2005 280 (30): 27595-603; Cullen, Mol Cell. 2004 16 (6): 861-5). The minimal length of the single-stranded flanks can easily be determined as when it becomes too short, the RNA molecule may lose its function because e.g. Drosha processing fails resulting in sequence specific inhibition being reduced or even absent. In some embodiments, the pri-miRNA scaffold carrying the first and second strand according to the invention has a 5′-sequence flank and a 3′ sequence flank relative to the predicted pre-miRNA structure of at least 5, at least 10, at least 15, at least 20, at least 30, at least 40, or at least 50 nucleotides. Preferably, the pri-miRNA derived flanking sequences (5′ and 3′) comprised in the miRNA scaffold are derived from the same naturally occurring pri-miRNA sequence. Preferably, pre-miRNA and/or the pri-miRNA derived flanking sequences (5′ and 3′) and/or loop sequences comprised in the miRNA scaffold are derived from the same naturally occurring pri-miRNA sequence. As the (putative) guide strand RNA as comprised in the endogenous miRNA sequence can be replaced by a sequence including (or consisting of) the first strand, and the passenger strand sequence replaced by a sequence including (or consisting of) the second strand, it is understood that flanking sequences and/or loop sequences of the pri-miRNA or pre-miRNA sequences of the endogenous sequence may include minor sequence modifications such that the predicted structure of the scaffold miRNA sequence (e.g. M-fold predicted structure) is the same as the predicted structure of the endogenous miRNA sequence.

The first and second strands and the third and fourth strands, such as to form two double stranded RNAs, i.e. the first and second RNA of the invention, are encoded by an expression cassette. It is understood that, unless otherwise stated, any additional RNAs comprised in the nucleic acid of the invention, such as the third RNA of the invention as described below, are also encoded by the expression cassette. It is also understood that when the double stranded RNAs are to be e.g. two siRNAs, consisting of two strands each, that there may be two or more expression cassettes required. When each double stranded RNA is comprised in a single RNA molecule, e.g. encoding a shRNA, pre-miRNA or pri-miRNA, one expression cassette per RNA molecule may suffice. A pol II expression cassette may comprise a promoter sequence a sequence encoding an RNA to be expressed followed by a polyadenylation sequence. In case the double stranded RNAs that are expressed comprise a pri-miRNA scaffold, the encoded RNA sequence may encode for intron sequences and exon sequences and 3′-UTR's and 5′-UTRs. A pol Ill expression cassette in general comprises a promoter sequence, followed by a sequence encoding an RNA (e.g. shRNA sequence, pre-miRNA, or a strand of the double stranded RNAs to be comprised in e.g. an siRNA or 5 extended siRNA). A pol I expression cassette may comprise a pol I promoter, followed by an RNA encoding sequence and a 3′-sequence. Expression cassettes for double stranded RNAs are well known in the art, and any type of expression cassette can suffice, e.g. one may use a pol III promoter, a pol II promoter or a pol I promoter (i.a. ter Brake et al., 2008 Mol Ther. Mar; 16 (3): 557-64, Maczuga et al. 2012 BMC Biotechnol. July 24; 12:42). In some embodiments, and as further described below, the expression cassette is a DNA molecule.

As is clear from the above, the first and second strands comprised in a double stranded RNA can contain additional nucleotides and/or nucleotide sequences. Any double stranded RNA of the invention may be comprised in a single RNA sequence or comprised in two separate RNA strands. Whatever design is used, it is designed such that, from the first and second RNA sequences, an antisense RNA molecule comprising the first or the second strand, as further explained below, in whole or a substantial part thereof, can be processed by the RNAi machinery, such that it is incorporated in the RISC complex to have its action, i.e. to induce RNAi against the RNA target sequence comprised in an RNA encoded by the c9orf72 gene. The sequence comprising or consisting of the first or second strand, in whole or a substantial part thereof, is capable of sequence specifically targeting RNA encoded by a human c9orf72 gene. Hence, as long as the double stranded RNA is capable of inducing RNAi, such a double stranded RNA is contemplated in the invention.

In some embodiments, the double stranded RNAs according to the invention are comprised in a pre-miRNA scaffold, a pri-miRNA scaffold, a shRNA, or an siRNA. Preferably, the first and second strand of said double stranded RNA encoded by the expressed cassette are to be contained in a single transcript. It is understood that the expressed transcript in subsequent processing, i.e. cleavage, results in the single transcript being processed into multiple separate RNA molecules.

The term complementary is herein defined herein as nucleotides of a nucleic acid sequence that can bind to another nucleic acid sequence through hydrogen bonds, i.e. nucleotides that are capable of base pairing. Ribonucleotides, the building blocks of RNA are composed of monomers (nucleotides) containing a sugar, phosphate and a base that is either a purine (guanine, adenine) or pyrimidine (uracil, cytosine). Complementary RNA strands form double stranded RNA. A double stranded RNA may be formed from two separate complementary RNA strands or the two complementary RNA strands may be comprised in one RNA strand. In complementary RNA strands, the nucleotides cytosine and guanine (C and G) can form a base pair, guanine and uracil (G and U), and uracil and adenine (U and A) can form a base pair as well. The term substantial complementarity means that is not require to have the first and second RNA sequence to be fully complementary, or to have the first RNA sequence and target RNA sequence or sequences of RNA encoded by the c9orf72 gene to be fully complementary.

miR-451

In some embodiments of the invention, the second RNA is incorporated in a pre-miRNA or a pri-miRNA scaffold derived from miR-451. In some embodiments of the invention, the second RNA comprises SEQ ID NO. 229 or a variant thereof.

As stated above, in some embodiments of the invention the nucleic acid comprises a sequence encoding a third RNA. In preferred embodiments, the third RNA comprises a hairpin and a guide sequence of at least 19 nucleotides substantially complementary to part of the c9orf72 gene, wherein the c9orf72 gene comprises a G4C2 hexanucleotide repeat expansion of at least 24 repeats in a non-coding region. In some specific embodiments, the third RNA is also incorporated in a pre-miRNA or a pri-miRNA scaffold derived from miR-451. In some embodiments of the invention, the third RNA comprises SEQ ID NO. 229 or a variant thereof.

Thus, and as shown in the examples, the third and fourth strands of the nucleic acid of the invention may be incorporated in a pre-miRNA or a pri-miRNA scaffold derived from miR-451. Similarly, also as shown in the examples, the fifth and sixth strands of the nucleic acid of the invention, where present, may also be incorporated in a pre-miRNA or a pri-miRNA scaffold derived from miR-451.

The miR-451 scaffold is found to be particularly useful within the present invention as it can induce RNA interference that can result in mainly guide strand induced RNA interference. The pri-miR-451 scaffold does not result in a passenger strand because the processing is different from the canonical miRNA processing pathway (Cheloufi et al. 2010 465 (7298): 584-9 and Yang et al., 2010 Proc Natl Acad Sci USA. 107 (34): 15163-8). The scaffolds represent an excellent candidate to develop a gene therapy product because unwanted potential off-targeting by passenger strands can be largely, if not completely, avoided. As the passenger strand (corresponding to the second sequence) may result in the targeting of transcripts other than RNA encoded by the gene of choice, using such scaffolds may prevent such unwanted targeting. Hence, it is preferred that selected scaffolds produce less than 15%; less than 10%; less than 5%; less than 4%; or less than 3% of passenger strands.

A miRNA-451 scaffold, as shown in the examples, and as shown in FIG. 3, preferably comprises from 5′ to 3′, firstly 5′-CUUGGGAAUGGCAAGG-3′ (SEQ ID NO. 231), followed by a sequence of 22 nucleotides, comprising or consisting of the first RNA sequence, followed by a sequence of 17 nucleotides, which can be regarded as the second RNA sequence, which is complementary over its entire length with nucleotides 2-18 of said sequence of 22 nucleotides, subsequently followed by sequence 5′-MWCUUGCUAUACCCAGA-3′ (wherein M is an A or a C or a G and W is an A or a U) (SEQ ID NO. 232). Preferably the first 5′-A/G/C nucleotide of the latter sequence is not to base pair with the first nucleotide of the first strand of the first or second RNA.

Such a scaffold may comprise further flanking sequences as found in the original pri-miR-451 scaffold. Alternatively, the hairpin stem sequences 5′-CUUGGGAAUGGCAAGG′-3′ (SEQ ID NO. 231) and 5′-MWCUUGCUAUACCCAGA-3′ (SEQ ID NO. 232) may be replaced by hairpin stem sequences of other pri-mRNA structures. As is clear from the above, the sequence of the scaffold may differ not only with regard to the (putative) guide strand sequence, and sequence complementary thereto, as present in the wild-type scaffold, but may also comprise additional mutations in the 5′ sequence, loop and 3′ sequence as well, as additional mutations may be required to provide for an RNA structure that is predicted to mimic the secondary structure of the wild-type scaffold. Such a scaffold may be comprised in a larger RNA transcript, e.g. a pol II expressed transcript, comprising e.g. a 5′ UTR and a 3′UTR and a poly A. Flanking structures may also be absent.

In some embodiments, an expression cassette in accordance with the invention thus encodes for a shRNA-like structure having a sequence of at least 22 nucleotides, comprising or consisting of the first strand of the RNA, followed by a sequence of 18 nucleotides, which can be regarded to be the second strand of the RNA, which is complementary over its entire length with nucleotides 2-18 of said sequence of 22 nucleotides. The latter shRNA-like structure derived from the miR-451 scaffold can be referred to as a pre-miRNA scaffold from miR-451. Alternatively, the flanking sequences, 5′-UGCCCKGGNN-3′ (SEQ ID NO. 233) and 5′-RSAUGGCA-3′ (SEQ ID NO. 234) may be replaced by flanking sequences of other pre-miRNA structures. Flanking structures may also be absent.

In some embodiments, an expression cassette according to the invention is provided, wherein the first strand of the second RNA and, where present, the first strand of the third RNA, is substantially complementary to a target RNA sequence comprised in antisense RNA transcripts encoded by the human c9orf72 gene. Preferably, said first strand of the second and third RNA is substantially complementary to a sequence selected from the group consisting of: SEQ ID NO. 1, 2, 3 or 90. More preferably said first strand of the second and third RNA has a length of 18, 19, 20, 21, or 22 nucleotides. More preferably, said first strand of the second and third RNA is fully complementary over its entire length with said target sequence. Most preferably, said first strand of the second or third RNA has a length of 19, 20, 21, or 22 nucleotides, wherein said first strand of the second and third RNA is fully complementary over its entire length with said target sequence.

As described below, the first strand of each of the second and third RNA is to be combined with a second strand of each of the second and third RNA. The skilled person is capable of designing and selecting a suitable second strand of the first and second RNA in order to provide for a first and second strand for the first and second RNA that can induce RNA interference when expressed in a cell.

Suitable first and second strands for the first, second and third RNA can be found in tables 1 and 2, respectively.

TABLE 1
Suitable first strands for the first,
second and third RNA.

| | Suitable first strands |
|---|---|
| SEQ ID NO. | (5′-sequence-3′) |
| 4 | CUAGCGGGACACCGUAGGUUAC |
| 5 | UCCUAGCGGGACACCGUAGGUU |
| 6 | ACGCACCUCUCUUUCCUAGCGG |
| 7 | UGACGCACCUCUCUUUCCUAGC |
| 8 | UUGACGCACCUCUCUUUCCUAG |
| 9 | UUUGACGCACCUCUCUUUCCUA |
| 10 | UCGCUGUUUGACGCACCUCUCU |
| 11 | UGUCGCUGUUUGACGCACCUCU |
| 12 | UUGUCGCUGUUUGACGCACCUC |
| 13 | ACUUGUCGCUGUUUGACGCACC |
| 14 | UACGUGGGCGGAACUUGUCGCU |
| 15 | UUACGUGGGCGGAACUUGUCGC |
| 16 | UUUACGUGGGCGGAACUUGUCG |
| 17 | UUUUACGUGGGCGGAACUUGUC |
| 18 | AGCGUCAUCUUUUACGUGGGCG |
| 19 | AAGCGUCAUCUUUUACGUGGGC |
| 20 | CAAGCGUCAUCUUUUACGUGGG |
| 21 | AGCGAGUACUGUGAGAGCAAGU |
| 22 | CAGCGAGUACUGUGAGAGCAAG |
| 23 | UCAGCGAGUACUGUGAGAGCAA |
| 24 | CACCCUCAGCGAGUACUGUGAG |
| 25 | UCACCCUCAGCGAGUACUGUGA |
| 26 | UUCACCCUCAGCGAGUACUGUG |
| 27 | GUUCACCCUCAGCGAGUACUGU |
| 28 | UGUUCACCCUCAGCGAGUACUG |
| 29 | CUUGUUCACCCUCAGCGAGUAC |
| 30 | UCUUGUUCACCCUCAGCGAGUA |
| 31 | UUCUUGUUCACCCUCAGCGAGU |
| 32 | AGGUCUUUUCUUGUUCACCCUC |
| 33 | CAGGUCUUUUCUUGUUCACCCU |
| 34 | UCAGGUCUUUUCUUGUUCACCC |
| 35 | AUCAGGUCUUUUCUUGUUCACC |
| 36 | UAUCAGGUCUUUUCUUGUUCAC |
| 37 | UUAUCAGGUCUUUUCUUGUUCA |
| 38 | CUUUAUCAGGUCUUUUCUUGUU |
| 39 | GGUUAAUCUUUAUCAGGUCUUU |
| 40 | UGGUUAAUCUUUAUCAGGUCUU |
| 41 | CUGGUUAAUCUUUAUCAGGUCU |
| 42 | UCUGGUUAAUCUUUAUCAGGUC |
| 43 | UUCUGGUUAAUCUUUAUCAGGU |
| 44 | UCCUUGUUUUCUUCUGGUUAAU |
| 45 | GCGGUUGUUUCCCUCCUUGUUU |
| 46 | UGCGGUUGUUUCCCUCCUUGUU |
| 47 | CUGCGGUUGUUUCCCUCCUUGU |
| 48 | GCUGCGGUUGUUUCCCUCCUUG |
| 49 | UGCUACAGGCUGCGGUUGUUUC |
| 50 | UUGCUACAGGCUGCGGUUGUUU |
| 51 | CUUGCUACAGGCUGCGGUUGUU |
| 52 | GAGCUUGCUACAGGCUGCGGUU |
| 53 | AGAGCUUGCUACAGGCUGCGGU |
| 54 | AGUUCCAGAGCUUGCUACAGGC |
| 55 | GAGUUCCAGAGCUUGCUACAGG |
| 56 | UGAGUUCCAGAGCUUGCUACAG |
| 57 | CUGAGUUCCAGAGCUUGCUACA |
| 58 | CGACUCCUGAGUUCCAGAGCUU |
| 59 | CGCGACUCCUGAGUUCCAGAGC |
| 60 | CGCGCGACUCCUGAGUUCCAGA |
| 61 | CCUAGCGCGCGACUCCUGAGUU |
| 62 | ACGUAAAAGAUGACGCUUGGUG |
| 63 | UCCGCCCACGUAAAAGAUGACG |
| 64 | CGGUGUCCCGCUAGGAAAGAGA |
| 65 | UGUGGGUUUAGGAGGUGUGUGU |
| 66 | AGCUCCGAGAUGACACAGACUU |
| 67 | UGCUUCACAGCUCCGAGAUGAC |
| 68 | ACAGACUUGCUUAAAGGAAGUG |

TABLE 2
Suitable second strands for the
second and third RNA.

| | Suitable second strands |
|---|---|
| SEQ ID NO. | (5′-sequence-3′) |
| 69 | CCUACGGUGUCCCGCUAC |
| 70 | UACGGUGUCCCGCUAGGC |
| 71 | UAGGAAAGAGAGGUGCGC |
| 72 | GGAAAGAGAGGUGCGUCC |
| 73 | GAAAGAGAGGUGCGUCAC |
| 74 | AAAGAGAGGUGCGUCAAC |
| 75 | AGGUGCGUCAAACAGCGC |
| 76 | GUGCGUCAAACAGCGACC |
| 77 | UGCGUCAAACAGCGACAC |
| 79 | CGUCAAACAGCGACAAGC |
| 80 | ACAAGUUCCGCCCACGUC |
| 81 | CAAGUUCCGCCCACGUAC |
| 82 | AAGUUCCGCCCACGUAAC |
| 83 | AGUUCCGCCCACGUAAAC |
| 84 | CACGUAAAAGAUGACGCC |
| 85 | ACGUAAAAGAUGACGCUC |
| 86 | CGUAAAAGAUGACGCUUC |
| 87 | GCUCUCACAGUACUCGCC |
| 88 | CUCUCACAGUACUCGCUC |
| 89 | UCUCACAGUACUCGCUGC |
| 90 | CAGUACUCGCUGAGGGUC |
| 91 | AGUACUCGCUGAGGGUGC |
| 92 | GUACUCGCUGAGGGUGAC |
| 93 | UACUCGCUGAGGGUGAAA |
| 94 | ACUCGCUGAGGGUGAACC |
| 95 | UCGCUGAGGGUGAACAAC |
| 96 | CGCUGAGGGUGAACAAGC |
| 97 | GCUGAGGGUGAACAAGAC |
| 98 | GUGAACAAGAAAAGACCC |
| 99 | UGAACAAGAAAAGACCUC |
| 100 | GAACAAGAAAAGACCUGC |
| 101 | AACAAGAAAAGACCUGAC |
| 102 | ACAAGAAAAGACCUGAUC |
| 103 | CAAGAAAAGACCUGAUAC |
| 104 | AGAAAAGACCUGAUAAAC |
| 105 | ACCUGAUAAAGAUUAACA |
| 106 | CCUGAUAAAGAUUAACCC |
| 107 | CUGAUAAAGAUUAACCAC |
| 108 | UGAUAAAGAUUAACCAGC |
| 109 | GAUAAAGAUUAACCAGAC |
| 110 | ACCAGAAGAAAACAAGGC |
| 111 | AAGGAGGGAAACAACCGA |
| 112 | AGGAGGGAAACAACCGCC |
| 113 | GGAGGGAAACAACCGCAC |
| 114 | GAGGGAAACAACCGCAGA |
| 115 | CAACCGCAGCCUGUAGCC |
| 116 | AACCGCAGCCUGUAGCAC |
| 117 | ACCGCAGCCUGUAGCAAC |
| 118 | GCAGCCUGUAGCAAGCUA |
| 119 | CAGCCUGUAGCAAGCUCC |
| 120 | GUAGCAAGCUCUGGAACC |
| 121 | UAGCAAGCUCUGGAACUA |
| 122 | AGCAAGCUCUGGAACUCC |
| 123 | GCAAGCUCUGGAACUCAC |
| 124 | UCUGGAACUCAGGAGUCC |
| 125 | UGGAACUCAGGAGUCGCC |
| 126 | GAACUCAGGAGUCGCGCC |
| 127 | CAGGAGUCGCGCGCUAGC |
| 128 | AAGCGUCAUCUUUUACGC |
| 129 | AUCUUUUACGUGGGCGGC |
| 130 | UUUCCUAGCGGGACACCC |
| 131 | ACACCUCCUAAACCCACC |
| 132 | CUGUGUCAUCUCGGAGCC |
| 133 | UCUCGGAGCUGUGAAGCC |
| 134 | UCCUUUAAGCAAGUCUGC |

Said first strand of the second and third RNA is preferably comprised in a miRNA scaffold, more preferably a miR-451 scaffold, such as shown in the examples. A suitable scaffold comprising a first and second strand for the second and third RNA in accordance with the invention can be a sequence such as SEQ ID NO 229.

Targeting these target RNA sequences, utilizing said first strand of the first and second RNA, were found to be particularly useful for reducing expression of antisense and sense RNA transcripts encoded by the human c9orf72 gene. By targeting human c9orf72 this way, the current inventors were able to efficiently reduce human c9orf72 gene expression and thus reduce the formation of RNA foci and dipeptide repeat proteins. Ultimately this may reverse, prevent, slow down the progression of, or completely halt neurodegeneration.

In some embodiments of the invention, the first RNA i.e., the first and second strands of the nucleic acid of the invention, is incorporated into a pre-miRNA or a pri-miRNA scaffold derived from miR-144. In some specific embodiments of the invention, the first RNA is incorporated into a miR-144 scaffold, and the second RNA is incorporated into a miR-451 scaffold.

As stated above, in certain embodiments, the first RNA comprises a first guide sequence. In those embodiments of the invention where the first RNA is incorporated into a miR-144 scaffold, the first guide sequence may be incorporated into the first or the second strand of the first RNA.

In some embodiments of the invention, the first RNA comprises a sequence selected from the group consisting of: SEQ ID NO. 181 or a variant thereof; SEQ ID NO. 230 or a variant thereof; and the second RNA comprises SEQ ID NO. 229 or a variant thereof. As stated above, the variants of SEQ ID NO. 181 comprise SEQ ID NO. 228.

In preferred embodiments of the invention, the first RNA comprises: SEQ ID NO. 230; a guide sequence of at least 19 nucleotides substantially complementary to a target sequence in a transcript encoded by a human c9orf72 gene; and the genomic sequence of the human c9orf72 gene comprises a G4C2 hexanucleotide repeat expansion in a non-coding region. In these embodiments, both the first and the second RNA comprise a guide sequence.

In some embodiments of the invention, the two or more guide sequences comprised in the RNAs of the invention are the same sequence. In other embodiments, the two or more guide sequences comprised in the RNAs of the invention are different sequences.

In those embodiments where the first RNA of the invention comprises a guide sequence, and the first RNA is incorporated into a miR-144 scaffold, the miRNA-144 scaffold for use in the invention preferably comprises from 5′ to 3′, firstly 5′-UGGGGCCCUGGCUM-3′ (wherein M is an A or a C or a G or a U) (SEQ ID NO. 235), followed by a sequence of 22 nucleotides, comprising or consisting of a first RNA sequence, followed by a sequence of 15 nucleotides which can be regarded as the apical loop 5′-UUUGCGAUGAGAWMM-3′ (wherein W is preferably a C or a G, but can also be a A or a U, and is to base pair with the last nucleotide of the first strand of the first RNA; and wherein M is an A or a C and is not to base pair with nucleotide 21 of the first strand of the first RNA), followed by a sequence of 20 nucleotides, which can be regarded as the second RNA sequence, and which is complementary over its entire length with nucleotides 1 and 3-10 and 12-20 of said sequence of 22 nucleotides, except nucleotide 18 that forms a mismatch with nucleotide 2 of the first strand, subsequently followed by sequence 5′-AGUCCGGGCACCCCC3′ (SEQ ID NO. 237).

Such a scaffold may comprise flanking sequences as found in the original pri-miR-144 scaffold. Alternatively, said flanking sequences may be replaced by flanking sequences of other pri-mRNA structures.

In some embodiments of the invention, the guide sequence is incorporated into the first strand of the first RNA. In some other embodiments of the invention, the guide sequence is incorporated into the second strand of the first RNA.

In those embodiments where the first RNA of the invention comprises a guide sequence, and the first RNA is incorporated into a miR-144 scaffold, the guide sequence may be comprised within the 5p- or 3p-arm of the scaffold. DROSHA processing of miR-144, subsequently followed by DICER processing, generate a miRNA duplex which ultimately enters the process of miRNA strand selection. Strand selection is operated within the RISC and determines which strand will become the active strand (also referred to as the guide strand) and which strand will be degraded (passenger strand) (Noland and Doudna, 2013 RNA, 19:639-648). Guide strand selection is highly determined by thermodynamic characteristics of the miRNA duplex, and thus it may be influenced by modifying the nucleotide sequence encoding the miRNA. Generally speaking, the strand with the lower thermodynamic stability at its 5′ end will become the guide strand. Another key feature of human miRNA guide strands is a 5′ end U-bias associated with an enrichment for A and G nucleotides, whilst the passenger strands exhibit a 5′ end C-bias and an enrichment for C and U nucleotides (Hai Yang Hu et al. 2009, BMC Genomics 2009, 10:413).

Conveniently, since the guide strand selection is sequence dependent, the miR-144 constructs of the invention comprising guide strands may be designed as 5p-or 3p-guide-containing regions (SEQ ID NO. 230). Importantly, the probability with which the desired guide strand of the invention will be selected in RISC can be modulated by single nucleotide variations such as selecting U as the first nucleotide of the first strand (5p-design) or the first nucleotide of the second strand (3p-designs) and selecting C or G as nucleotide 20 of the first strand (5p-designs) or nucleotide 19 of the second strand (3p-designs).

As with miR-451 scaffolds, the sequence of the miR-144 scaffold may differ not only with regard to the (putative) guide strand sequence, and sequence complementary thereto, as present in the wild-type scaffold, but may also comprise additional mutations in the 5′, loop and 3′ sequence as well, as additional mutations may be required to provide for an RNA structure that is predicted to mimic the secondary structure of the wild-type scaffold. Again, such a scaffold may be comprised in a larger RNA transcript, e.g. a pol II expressed transcript, comprising e.g. a 5′ UTR and a 3′UTR and a poly A. Flanking structures may also be absent. An expression cassette in accordance with the invention may thus express a shRNA-like structure having a sequence of 22 nucleotides, comprising or consisting of the first strand of the first RNA, followed by a sequence of 17 nucleotides, which can be regarded to be the second strand of the first RNA, which is complementary over its entire length with nucleotides 1 and 3-10 and 11-19 of said sequence of 22 nucleotides, except nucleotide 18 that forms a mismatch with nucleotide 2 of the first strand. The latter shRNA-like structure derived from the miR-144 scaffold can be referred to as a pre-miRNA scaffold from miR-144. Alternatively, the flanking sequences, 5′-ATCGGCGCTATGCTTCCTGTGCCCCCAG-3′ (SEQ ID NO. 237) and 5′-AGCTCTGGAGCCTGACAAGGAGGACAGGAGAGATGCTGCAAGCCCAAGAAGCTCTCTGCTC AGCCTGTCACAACCTACTGACTGCCAGGGCA-3′ (SEQ ID NO. 225) may be replaced by flanking sequences of other pri-mRNA structures. Flanking structures may also be absent.

In some embodiments, an expression cassette according to the invention is provided, wherein said first or second strand of the first RNA is substantially complementary to a target RNA sequence comprised in antisense RNA transcripts encoded by the human c9orf72 gene.

In some embodiments, the first or the second strand of the first RNA is substantially complementary to a sequence selected from the group consisting of: SEQ ID NO. 1, 2, 3 or 90. In some preferred embodiments, the first or the second strand of the first RNA has a length of 18, 19, 20, 21, or 22 nucleotides. In some specific embodiments, the first or second strand of the first RNA is fully complementary over its entire length with the target sequence. In some preferred embodiments, the first or the second strand of the second RNA has a length of 19, 20, 21, or 22 nucleotides, wherein said first strand of the second RNA is fully complementary over its entire length with the target sequence. The first or strand of the second RNA can be selected from the group consisting of SEQ ID NO. 4 to SEQ ID NO. 134, as disclosed in Tables 1 and 2.

As explained above, where the first strand of the first RNA comprises a guide sequence, the first strand is to be combined with a second strand of the first RNA, which may also be referred to as the first and second strands of the invention. As above, the skilled person is capable of designing and selecting a suitable second strand of the first RNA; suitable second strands of the first RNA that can be contemplated are listed in Table 1.

Similarly, where the second strand of the first RNA comprises a guide sequence, the second strand is to be combined with a first strand of the first RNA, which may again be referred to as the first and second strands of the invention. As above, the skilled person is well capable of designing and selecting a suitable first strand of the first RNA, with suitable first strands of the first RNA listed in Table 1.

As above, targeting these target RNA sequences, utilizing such first or second strand of the first RNA and such first strand of the second RNA, was found to be in particular useful for reducing expression of antisense and sense RNA transcripts encoded by the human c9orf72 gene.

A suitable scaffold comprising a first and second strand for the first RNA in accordance with the invention can be a sequence such as SEQ ID NO. 230.

Thus, in some embodiments of the invention, the first RNA comprises SEQ ID NO. 230 or a variant thereof. In some more specific embodiments of the invention, the first RNA comprises SEQ ID NO. 230 or a variant thereof; and the second RNA comprises SEQ ID NO. 229 or a variant thereof.

### Guide Strands

The first, second and third RNA to be expressed in accordance with the invention may comprise, in whole or a substantial part thereof, a guide strand. For convenience and clarity, the invention will be explained for a RNA below; the same embodiments apply to the first, second and third RNA when they comprise a guide strand.

In certain embodiments of the invention, the guide strand may also be referred to as antisense strand as it is complementary (“anti”) to a target RNA sequence in a sense or antisense transcript, the sense or antisense target RNA sequence being comprised in an RNA encoded by a gene of choice. Thus, the first RNA also comprise a “sense strand”, that may have substantial sequence identity with, or be identical to, the target RNA sequence. Therefore, the first RNA can be described as a hairpin or a double stranded RNA that is substantially complementary to itself.

Said double stranded RNA according to the invention is to induce RNA interference, thereby reducing expression of c9orf72 transcripts, which includes knocking down of c9orf72 derived transcripts. Transcripts that may be targeted may include splice variants, and unspliced RNA transcripts. Thus, an RNA encoded by a human c9orf72 gene is understood to comprise unspliced mRNAs comprising a 5′ untranslated region (UTR), intron and exon sequences, followed by a 3′ UTR and a polyA signal, and also splice variants thereof. The double stranded RNA according to the invention may also induce transcriptional silencing.

Reducing expression of c9orf72 transcripts is herein thus preferably understood as reducing the steady state level of any transcript encoded by the c9orf72 gene in a target cell such that less of the transcripts is available in the cell for translation, thereby reducing the steady state level of the c9orf72 protein in the target cell. Reducing expression of a transcript encoded by the c9orf72 gene therefore does not necessarily involve reducing de novo transcription of the gene but rather increased degradation of a transcript encoded by the c9orf72 gene.

In some embodiments, the double stranded RNA according to the invention comprises a first RNA sequence and a second RNA sequence, wherein the first and second RNA sequence are substantially complementary, and wherein the first RNA sequence has a sequence length of at least 19 nucleotides and is substantially complementary to a target RNA sequence of an RNA encoded by the c9orf72 gene, which first RNA sequence is capable of inducing RNA interference to sequence-specifically reduce expression of an RNA transcript comprising the target RNA sequence. In some further embodiments, said induction of RNA interference to reduce expression of an RNA transcript comprising the target RNA sequence means that it is to reduce c9orf72 gene expression.

Similarly, in some embodiments, the double stranded RNA according to the invention comprises a first RNA sequence and a second RNA sequence, wherein the first and second RNA sequences are substantially complementary, and wherein the second RNA sequence has a sequence length of at least 19 nucleotides and is substantially complementary to a target RNA sequence of an RNA encoded by a first or second gene, which first RNA sequence is capable of inducing RNA interference to sequence-specifically reduce expression of an RNA transcript comprising the target RNA sequence. In some further embodiments, said induction of RNA interference to reduce expression of an RNA transcript comprising the target RNA sequence means that it is to reduce the first and second gene expression.

The skilled person can easily determine whether this is the case by using standard luciferase reporter assays and appropriate controls such as described in the examples and as known in the art (Zhuang et al. 2006 Methods Mol Biol.342:181-7). For example, a luciferase reporter comprising a target RNA sequence can be used to show that the double stranded RNA according to the invention is capable of sequence specific knock down. Furthermore, levels of c9orf72 expression can be determined by detecting c9orf72 mRNA (nuclear and/or cytoplasmic), c9orf72 protein, reduction of formed RNA foci (sense and/or antisense) and reduction of dipeptide repeat proteins (soluble and/or insoluble).

As used herein, the term “RNA silencing” refers to a group of sequence-specific regulatory mechanisms (e.g. RNA interference (RNAi), transcriptional gene silencing (TGS), post-transcriptional gene silencing (PTGS), quelling, co-suppression, and translational repression) mediated by RNA molecules which result in the inhibition or “silencing” of the expression of a corresponding protein-coding gene. RNA silencing has been observed in many types of organisms, including plants, animals, and fungi.

It is understood that “substantially complementary” in this context means that it is not required to have all the nucleotides of the guide sequence and the target sequence to be base paired, i.e. to be fully complementary, or all the nucleotides of the guide sequence and the target sequence to be base paired. As long as the second RNA is capable of inducing RNA interference to thereby sequence-specifically target a sequence comprising the target RNA sequence, such substantial complementarity is contemplated in accordance with the invention.

The substantial complementarity between the strand complementary to the target RNA sequence, also referred to as part of the c9orf72 gene, preferably consists of at most two mismatched nucleotides, more preferably having one mismatched nucleotide, most preferably having no mismatches. It is understood that one mismatched nucleotide means that over the entire length of the strand complementary to the target RNA sequence when base paired with the target RNA sequence one nucleotide does not base pair with the target RNA sequence. Having no mismatches means that all nucleotides of the strand complementary to the target RNA base pair with the target RNA sequence, having 2 mismatches means two nucleotides of the strand complementary to the target RNA do not base pair with the target RNA sequence. The strand complementary to the target RNA may also comprise additional nucleotides that do not have complementarity to the target RNA sequence, and may be longer than e.g. 21 nucleotides. In such a scenario, the substantial complementarity is determined over the entire length of the target RNA sequence. This means that the target RNA sequence in this embodiment has either no, one or two mismatches over its entire length when base paired with the strand complementary to the target RNA.

As shown in the example section, double stranded RNAs comprising a strand complementary to the target RNA length of 22 nucleotides were tested. These strands complementary to the target RNA had no mismatches and were fully complementary with the target RNA sequence. Having a few mismatches between the strand complementary to the target RNA and the target RNA sequence may however be allowed according to the invention, as long as the double stranded RNA according to the invention is capable of reducing expression of transcripts comprising the target RNA sequence, such as a luciferase reporter or e.g. a transcript comprising the target RNA sequence.

Substantial complementarity between the strand complementary to the target RNA and the target RNA sequence consists of having no, one or two mismatches over the entire length of either the strand complementary to the target RNA or the target RNA sequence encoded by an RNA of the human c9orf72, whichever is the shortest.

As said, a mismatch according to the invention means that a nucleotide of the first (or second) and third strand (the strand) does not base pair with the target RNA sequence encoded by an RNA of the first or second gene. Nucleotides that do not base pair are A and A, G and G, C and C, U and U, A and C, C and U, or A and G. A mismatch may also result from a deletion of a nucleotide, or an insertion of a nucleotide. When the mismatch is a deletion in the strand sequence, this means that a nucleotide of the target RNA sequence is not base paired with the sequence when compared with the entire length of the strand sequence. Nucleotides that can base pair are A-U, G-C and G-U. A G-U base pair is also referred to as a G-U wobble, or wobble base pair. In one embodiment the number of G-U base pairs between the strand sequence and the target RNA sequence is 0, 1 or 2.

In some embodiments, there are no mismatches between the strand RNA sequence and the target RNA sequence and a G-U base pair or G-U pairs are allowed. Preferably, there may be no G-U base pairs between the strand sequence and the target RNA sequence, or the strand sequence and the target RNA sequence only have base pairs that are A-U or G-C. Preferably, there are no G-U base pairs and no mismatches between the RNA strand sequence and the target RNA sequence. The strand sequence of the double stranded RNA according to invention preferably is fully complementary to the target RNA sequence, said complementarity consisting of G-U, G-C and A-U base pairs. The strand sequence of the double stranded RNA according to invention more preferably is fully complementary to the target RNA sequence, said complementarity consisting of G-C and A-U base pairs.

Therefore, in some embodiments, the strand sequence and the target RNA sequence have at least 15, 16, 17, 18, or 19 nucleotides that base pair. Preferably the strand and the target RNA sequence are substantially complementary, said complementarity comprising at least 19 base pairs. In other embodiments, the strand has at least 8, 9, 10, 11, 12, 13 or 14 consecutive nucleotides that base pair with consecutive nucleotides of the target RNA sequence. In other embodiments, the strand has at least 19 consecutive nucleotides that base pair with consecutive nucleotides of the target RNA sequence. In other embodiments, the strand comprises at least 19 consecutive nucleotides that base pair with 19 consecutive nucleotides of the target RNA sequence. In other embodiments, the strand has at least 17 nucleotides that base pair with the target RNA sequence and have at least 15 consecutive nucleotides that base pair with consecutive nucleotides of the target RNA sequence. The sequence length of the first strand is preferably at most 21, 22, 23, 24, 25, 26, or 27 nucleotides. In other embodiments, the strand has at least 20 consecutive nucleotides that base pair with 20 consecutive nucleotides of the target RNA sequence. In other embodiments, the strand comprises at least 21 consecutive nucleotides that base pair with 21 consecutive nucleotides of the target RNA sequence.

As said, it may be not required to have full complementarity (i.e. full base pairing (no mismatches) and no G-U base pairs) between the first or second strand of the first RNA and the first strand of the second RNA, and the target RNA sequence as such a strand can still allow for sufficient suppression of gene expression. Also, not having full complementarity may be contemplated for example to avoid or reduce off-target RNA sequence specific gene suppression while maintaining sequence specific inhibition of transcripts comprising the target RNA sequence. However, it may be preferred to have full complementarity as it may result in more potent inhibition. Without being bound by theory, having full complementarity between the first or second strand of the first RNA and the first strand of the second RNA, and the target RNA sequence may allow for the activated RISC complex comprising said first or second strand of the first RNA or the first strand of the second RNA (or a substantial part thereof) to cleave its target RNA sequence, whereas having mismatches may hamper cleavage and can result in mainly allowing inhibition of translation, of which the latter may result in less potent inhibition.

With regard to the second strand on the RNA of the invention, the second strand is substantially complementary with the first strand on the RNA. The second strand combined with the first strand forms a double stranded RNA. As said, this is to form a suitable substrate for the RNA interference machinery such that a guide sequence derived from the first strand is comprised in the RISC complex in order to sequence specifically inhibit expression of its target, i.e. RNA encoded by the first or second gene. The sequence of the second strand has sequence similarity with the target RNA sequence. However, the substantial complementarity of the second strand with the first strand may be selected to have less substantial complementarity as compared with the substantial complementarity between the first strand and the target RNA sequence. Hence, the second strand may comprise 0, 1, 2, 3, 4, or more mismatches, 0, 1, 2, 3, or more GU wobble base pairs, and may comprise insertions of 0, 1, 2, 3, 4, nucleotides and/or deletions of 0, 1, 2, 3, 4, nucleotides. It is understood that, where the guide sequence is comprised within the second strand of the first RNA, or the second strand of the invention, the description above applies to the first strand of the invention.

Preferably the first strand and the second strand are substantially complementary, said complementarity comprising 0, 1, 2 or 3 G U base pairs and/or wherein said complementarity comprises at least 17 base pairs. These mismatches, G-U wobble base pairs, insertions and deletions, are with regard to the first strand, i.e. the double stranded region that is formed between the first and second strands. As long as the first and second strands can substantially base pair, and are capable of inducing sequence specific inhibition of an RNA encoded by the first or second gene, such substantial complementarity is allowed according to the invention. It is also understood that substantially complementarity between the first and the second strands may depend on the double stranded RNA design of choice. It may depend for example on the miRNA scaffold that is chosen for in which the double stranded RNA is to be incorporated

As clear from the above, the substantial complementarity between the first strand and second strand of the RNA may comprise mismatches, deletions and/or insertions relative to a first and second RNA sequence being fully complementary (i.e. fully base paired). In some embodiments, the first and second strands of the RNA have at least 11 consecutive base pairs. Hence, at least 11 consecutive nucleotides of the first strand and at least 11 consecutive nucleotides of the second strand are fully complementary. In some embodiment the first and second strands of the RNA have at least 15 nucleotides that base pair. Said base pairing between at least 15 nucleotides of the first strand and at least 15 nucleotides of the second strand may consist of G-U, G-C and A-U base pairs, or may consist of G-C and A-U base pairs.

In some embodiments, the first and second RNA sequences have at least 15 nucleotides that base pair and have at least 11 consecutive base pairs. In another embodiments, the first RNA sequence and the second RNA sequence are substantially complementary, wherein said complementarity comprises at least 17 base pairs. Said 17 base pairs may preferably be 17 consecutive base pairs, said base pairing consisting of G-U, G-C and A-U base pairs or consisting of G-C and A-U base pairs.

The invention thus provides for an expression cassette encoding the first strand and second strand of the RNA wherein the first and second strands are substantially complementary, wherein the first strand has a sequence length of at least 19 nucleotides and is substantially complementary to a target RNA sequence comprised in an RNA encoded by a human c9orf72 gene.

For clarity, since the embodiments are described in reference to an RNA, the invention thus provides for an expression cassette encoding: the first strand and second strand of the first RNA wherein the first and second strands are substantially complementary, wherein the first or second strand has a sequence length of at least 19 nucleotides and is substantially complementary to a target RNA sequence comprised in an RNA encoded by the c9orf72 gene; and the first strand and second strand of the second RNA wherein the first and second strands are substantially complementary, wherein the first strand has a sequence length of at least 19 nucleotides and is substantially complementary to a target RNA sequence comprised in an RNA encoded by the c9orf72 gene.

The invention also provides for an expression cassette encoding: the first strand and second strand of the first RNA wherein the first and second strands are substantially complementary; the first strand and second strand of the second RNA wherein the first and second strands are substantially complementary, wherein the first strand has a sequence length of at least 19 nucleotides and is substantially complementary to a target RNA sequence comprised in an RNA encoded by a human c9orf72 gene; and the first strand and second strand of the third RNA wherein the first and second strands are substantially complementary, wherein the first strand has a sequence length of at least 19 nucleotides and is substantially complementary to a target RNA sequence comprised in an RNA encoded by a human c9orf72 gene.

In some embodiments of the invention, each of the guide sequences is substantially complementary to a sequence selected from the group consisting of: SEQ ID NO. 1, 2, 3 or 90.

As stated above, in some embodiments of the invention, the first and second guide sequences of the invention are the same sequence. In other embodiments, the first and second guide sequences of the invention are different sequences.

The first, second and third RNA of the invention may be incorporated into miRNA scaffolds. The miRNA scaffold sequence is processed by the RNAi machinery as present in the cell. In some specific embodiments of the invention, the processing of the miRNA scaffold sequence results in: guide sequences comprising the first strand of the first RNA, or a substantial part thereof, in the range of 21-30 nucleotides; and guide sequences comprising the first strand of the second RNA, or a substantial part thereof, in the range of 21-30 nucleotides. In some other specific embodiments of the invention, the processing of the miRNA scaffold sequence results in: guide sequences comprising the second strand of the first RNA, or a substantial part thereof, in the range of 21-30 nucleotides; and guide sequences comprising the first strand of the second RNA, or a substantial part thereof, in the range of 21-30 nucleotides.

Such guide strands are capable of reducing c9orf72 transcript expression by targeting the selected target sequences.

As shown in the examples, the first strand of the first RNA and the first strand of the second or third RNA, or, alternatively, the second strand of the first RNA and the first strand of the second and third RNA, as it is encoded by the expression cassette of the invention, is comprised in part or in whole, in a guide strand when it has been processed by the RNAi machinery of the cell. Hence, the guide strand that is to be generated from the RNA encoded by the expression cassette, comprising the first or second strand of the first RNA and the first strand of the second RNA, is to comprise at least 18 nucleotides of the second RNA sequence of the double stranded RNA.

Preferably, such a guide strand comprises at least 19 nucleotides, 20 nucleotides, 21 nucleotides, or 22 nucleotides. A guide strand can comprise the first or second strand of the first or the first strand of the second RNA sequence, also as a whole. In selecting a miRNA scaffold, the first or second strand of the first RNA sequence and the first strand in the second RNA sequence, can be selected such that it is to replace the original guide strand. However, this does not necessarily mean that the guide strand produced from such an artificial scaffold is identical in length to the first or second strand of the first RNA or the first strand of the second RNA selected, nor that the first or second strand of the first RNA or the first strand of the second RNA is in its entirety to be found in the guide strand that is produced.

In a second aspect of the invention, there is provided an expression cassette comprising the nucleic acid of the invention, wherein the expression cassette is a DNA molecule. In some specific embodiments, the nucleic acid comprised within the cassette is operably linked to a promoter and optionally to a poly-A signal. In some embodiments, the expression cassette comprises, in 5′ to 3′ order, the following elements: at least one promoter, at least a first and second RNA and at least a poly A signal.

The nucleotide sequence comprising an expression cassette or expression cassettes as defined above for expression in a mammalian cell comprises at least one mammalian cell-compatible expression control sequence, e.g. a promoter, that is/are operably linked to the sequence coding for the gene product of interest, thus forming an expression cassette for expression of the gene product of interest in mammalian target cell to be treated by gene therapy with the gene product of interest. Many such promoters are known in the art (see Sambrook and Russel, 2001, supra).

Constitutive promoters that are broadly expressed in many cell-types, such as the CMV promoter, may be used. However, more preferred will be promoters that are inducible, tissue-specific, cell-type-specific, or cell cycle-specific. Preferably a pol II promoter is used, such as a CAG promoter (i.a. Miyazaki et al. 1989 Gene 79 (2): 269-77; Niwa N. et al. 1991 Gene. 108 (2): 193-9), a PGK promoter, or a CMV promoter (Such as depicted e.g. in FIG. 2 of WO2016102664). For any diseases primarily affect the brain, it may be particularly useful to use a neurospecific promoter.

Thus, in some embodiments of the invention, the promoter is a promoter capable of driving transcription in a brain cell. Examples of suitable neuron-specific promoters are Neuron-Specific Enolase (NSE), human synapsin 1 (hSYN), calcium-calmodulin-dependent protein kinase Il (CaMKII), tubulin a| (Tα1), platelet-derived growth factor B-chain (PDGF-β), methyl-CpG binding protein 2 (MeCP2) (Hioki et al. 2007 Gene Ther. 14 (11): 872-82; Powell S K et al. 2015 Discov Med. 19 (102): 49-57). Examples of suitable ubiquitous promoters are cytomegalovirus (CMV), chicken β-actin (CBA), CMV early enhancer/chicken β-actin (CAG), hybrid forms of the CBA, human elongation factor-1α (EF-1α), Rous sarcoma virus (RSV), phosphoglycerate kinase 1 promoter (PGK), β glucuronidase (GUSB), and ubiquitin C (UBC). Other suitable promoters that can be contemplated are inducible promoters, i.e. a promoter that initiates transcription only when the host cell is exposed to some particular stimulus.

The expression cassette comprising the nucleic acid of the invention encodes a polyA signal. In some embodiments of the invention, the polyadenylation (polyA) tail is the simian virus 40 polyA (SV40 polyA), the bovine growth hormone polyA (bGH polyA), the human growth hormone polyA (hGH polyA), or the synthetic polyA (spA polyA) from rabbit β-globin (Powell S K et al., supra).

The expression cassettes according to the invention can be transferred to a cell, using e.g. transfection methods. Any suitable means may suffice to transfer an expression cassette according to the invention. Preferably, the expression cassettes according to the invention are comprised in a viral vector, preferably a gene therapy vector. Preferably, gene therapy vectors are used that stably transfer the expression cassette to the cells such that stable expression of the double stranded RNAs that induce sequence specific inhibition of the first and second gene can be achieved. Suitable vectors may be lentiviral vectors, retrotransposon based vector systems, SV40 vectors, Adenoviral vectors, Vaccinia virus vectors, Herpes simplex virus vectors or AAV vectors. It is understood that as e.g. lentiviral vectors carry an RNA genome, the RNA genome will encode for the said expression cassette such that after transduction of a cell, the said DNA sequence and said expression cassette is formed. Preferably, the gene therapy vector is a viral vector. Preferably the viral vector is an AAV. Therefore, in some embodiments, an expression cassette as disclosed herein is flanked by Inverted Terminal Repeats.

Thus, in preferred embodiments of the invention, the expression cassette comprising the nucleic acid of the invention is flanked by at least one AAV Inverted Terminal Repeat (ITR). In some specific embodiments, the expression cassette is flanked by one 5′ ITR and one 3′ ITR. In other words, the expression cassette is flanked by an ITR sequence at the 5′ end of the cassette and an ITR sequence at the 3′ end of the cassette.

### AAVs

A third aspect of the present invention relates to an adeno-associated virus (AAV) comprising the expression cassette of the invention.

In some embodiments of the invention, the AAV comprises the expression cassette of the invention.

Recombinant parvoviruses, in particular dependoviruses such as infectious human or simian adeno-associated virus (AAV), and the components thereof (e.g. a parvovirus genome), may be used as vectors for introduction and/or expression of nucleic acids in mammalian cells, preferably human cells. An AAV vector is defined as a recombinantly produced AAV or AAV particle that comprises a polynucleotide to be delivered into a host cell, either in vivo, ex vivo or in vitro. Herein, an AAV vector construct refers to the polynucleotide comprising the viral genome or part thereof, usually at least one ITR, and a transgene. Herein, a transgene refers to a nucleotide sequence of interest and may comprise promotor and/or regulatory sequences necessary for expression as well as sequences encoding the gene of interest.

A “recombinant parvoviral or AAV vector” (or “rAAV vector”) or a “parvoviral or AAV vector” herein refers to a parvoviral or AAV virion (i.e. a capsid), comprising (or “packaging”) one or more nucleotide sequences of interest, genes of interest or “transgenes” that is/are flanked by at least one parvoviral or AAV inverted terminal repeat sequence (ITR). Preferably, the transgene(s) is/are flanked by ITRs, one on each side of the transgene(s). Such (r) AAV vectors can be replicated and packaged into infectious viral particles when present in a suitable host cell that is expressing AAV rep and cap gene products (i.e. AAV Rep and Cap proteins). When the transgene(s) of interest that is/are flanked by at least one ITR is incorporated into a larger nucleic acid construct (e.g. in a chromosome or in another vector such as a plasmid or baculovirus used for cloning or transfection), this is typically referred to as a “pro-vector” which can be “rescued” by replication and encapsidation in the presence of AAV packaging functions and necessary helper functions.

Preferably the AAV vector that is used is an AAV vector of serotype 5 or serotype 9. AAV of serotype 5 or 9 (also referred to as AAV5 and AAV9) may be in particularly useful for transducing human neurons and human astrocytes such as shown in the examples. Therefore, in some embodiment, there is an AAV comprising an expression cassette as disclosed herein. Thus, AAV5 and AAV9 can efficiently transduce different human cell types of the CNS including FBN, dopaminergic neurons, motor neurons and astrocytes and is therefore a suitable vector candidate to deliver therapeutic genes to the CNS to treat neurogenerative diseases, including but not limited to the treatment of ALS and/or FTD, via targeting e.g. the first and second gene as described herein. The production of AAV vectors comprising any expression cassette of interest is well described in: WO2007/046703, WO2007/148971, WO2009/014445, WO2009/104964, WO2011/122950, WO2013/0361 18, which are incorporated herein in their entirety.

AAV sequences that may be used in the present invention for the production of AAV vectors, e.g. produced in insect or mammalian cell lines, can be derived from the genome of any AAV serotype. Generally, the AAV serotypes have genomic sequences of significant homology at the amino acid and the nucleic acid levels, provide an identical set of genetic functions, produce virions that are essentially physically and functionally equivalent, and replicate and assemble by practically identical mechanisms. For the genomic sequence of the various AAV serotypes and an overview of the genomic similarities see e.g. GenBank Accession number U89790; GenBank Accession number J01901; GenBank Accession number AF043303; GenBank Accession number AF085716; Chlorini et al. (1997, J. Vir. 71:6823-33); Srivastava et al. (1983, J. Vir. 45:555-64); Chlorini et al. (1999, J. Vir. 73:1309-1319); Rutledge et al. (1998, J. Vir. 72:309-319); and Wu et al. (2000, J. Vir. 74:8635-47). AAV serotypes 1, 2, 3, 4 and 5 are a preferred source of AAV nucleotide sequences for use in the context of the present invention. Preferably the AAV ITR sequences for use in the context of the present invention are derived from AAV1, AAV2, and/or AAV5. Likewise, the Rep52, Rep40, Rep78 and/or Rep68 coding sequences are preferably derived from AAV1, AAV2 and AAV5. The sequences coding for the VP1, VP2, and VP3 capsid proteins for use in the context of the present invention may however be taken from any of the known 42 serotypes, more preferably from AAV1, AAV2, AAV3, AAV4, AAV5, AAV6, AAV7, AAV8 or AAV9 or newly developed AAV-like particles obtained by e.g. capsid shuffling techniques and AAV capsid libraries. AAV capsids may consist of VP1, VP2 and VP3, but may also consist of VP1 and VP3.

In some embodiments, the AAV vector according to the inventions comprises AAV5 or AAV9 capsid proteins. In some embodiment, the AAV vector according to the inventions comprises an AAV5 capsid protein. In some embodiment, the AAV vector according to the inventions comprises an AAV9 capsid protein.

In another embodiment, a host cell is provided comprising the said nucleic acid or said expression cassette according to the invention. For example, the said expression cassette or nucleic acid may be comprised in a plasmid contained in bacteria. Said expression cassette or nucleic acid may also be comprised in a production cell that produces e.g. a viral vector. Said expression cassette may also be provided in a baculovirus vector.

Various modifications of the nucleotide sequences as defined above, including e.g. the wildtype AAV sequences, for proper expression in the host cell is achieved by application of well-known genetic engineering techniques such as described e.g. in Sambrook and Russell (2001, supra). Various further modifications of coding regions are known to the skilled artisan which could increase the yield of the encoded proteins. These modifications are within the scope of the present invention.

In one embodiment, any mammalian cell may be infected by an AAV vector of the invention, for example, but not limited to, a muscle cell, a liver cell, a nerve cell, a glial cell and an epithelial cell; and the mammalian species may be any species including, but not limited to, murine, dog, non-human primate and human. In a preferred embodiment the cell to be infected is a human cell.

It is to be understood that the capsid amino acid sequences and the nucleotide sequences encoding them can be engineered, for example, the sequence may be a hybrid form or may be codon optimized, such as for example by codon usage of AcmNPv or Spodoptera frugiperda. The capsid proteins may be engineered, for example, via directed evolution, DNA shuffling, error prone PCR, bioinformatics rational design or site saturated mutagenesis. Resulting capsids are based on the existing serotypes but contain various amino acid or nucleotide changes that improve the features of such capsids. The resulting capsids can be a combination of various parts of existing serotypes, “shuffled capsids” or contain completely novel changes, i.e. additions, deletions or substitutions of one or more amino acids or nucleotides, organized in groups or spread over the whole length of gene or protein. See for example Schaffer and Maheshri; Proceedings of the 26th Annual International Conference of the IEEE EMBS San Francisco, CA, USA; Sep. 1-5, 2004, pages 3520-3523; Asuri et al., 2012, Molecular Therapy 20 (2): 329-3389; Lisowski et al., 2014, Nature 506 (7488): 382-386, herein incorporated by reference.

In some embodiments, the ITRs and capsid proteins (or parts thereof) in the AAV vector of the invention may be from different AAV serotypes. By way of example, and not limitation, the ITRs may be derived from AAV2, whilst the capsid proteins may be derived from a different serotype, for example AAV5 or AAV9.

In a fourth aspect, the present invention relates to a pharmaceutical composition (“the pharmaceutical composition of the invention”) comprising the nucleic acid of the invention, the expression cassette of the invention, the AAV vector of the invention, and at least one pharmaceutically acceptable excipient.

Suitable excipients include, but are not limited to, buffers and stabilizers, antioxidants, etc. By way of example, the pharmaceutical composition of the invention may comprise physiological buffers, such as e.g. PBS, and stabilizing agents such as e.g. sucrose.

The compositions of the invention are compatible with and suitable and intended for use in subsequent intravenous, intrastriatal, intrathecal, intracerebellar, intraparenchymal, intracerobroventricular, intracisternal, intraportal, intravitreal, subretinal administration or for use in organ-targeted vascular delivery such as intraporal or intracoronary delivery or isolated limb perfusion.

In some embodiments, the pharmaceutical composition of the invention may also comprise at least one immunosuppressive compound. Said compound may reduce and/or prevent an immune response induced by administration of the pharmaceutical composition of the invention.

In some embodiments these compositions are used to transduce cells in vitro or ex vivo, in which case the excipients will need to be compatible with cell culture.

In a fifth aspect, the invention relates to the nucleic acid of the invention, the expression cassette of the invention, the AAV vector of the invention, or the pharmaceutical composition of the invention for use as a medicament. The appropriate definitions are provided elsewhere in this application.

In some embodiments, said medicament reduces expression of RNA encoded by the human c9orf72 gene, as explained herein above.

In some embodiments of the invention, the medicament is used for treating and/or preventing amyotrophic lateral sclerosis (ALS) and/or frontotemporal dementia (FTD), or any atypical presentations mimicking other kinds of neurodegenerative brain diseases.

As used herein, the term “treat” and any of its variants refers to any kind of healthcare which is intended to relieve or eliminate the symptoms and/or causes of illness, injury, mental health problems, etc. The term “prevent” and any of its variants refers to any action taken to decrease the chance of getting a disease or condition.

It is understood that treatment of ALS, FTD and any atypical presentations mimicking other kinds of neurodegenerative brain diseases may be provided not only to human subjects suffering from any of said diseases, but also to human subjects having a genetic predisposition of developing the diseases who may or may not show symptoms of the disease. Thus, treatment of human subjects with ALS, FTD, or any atypical presentations mimicking other kinds of neurodegenerative brain diseases includes the treatment of any human subject carrying an c9orf72 gene which sequence comprises over 24 G4C2 hexanucleotide repeats.

In some specific embodiments of the invention, the first and the second target sequences are the same. In said embodiments, the potency of the medicament is maximized when compared to equivalent medicaments comprising AAVs bearing only one RNA sequence targeting the said same sequence.

Within the context of this invention, “potency” is understood as an expression of the activity of a medicament or therapeutic substance, in terms of the concentration or amount needed to produce a defined effect. The potency of a given medicament may be expressed as the concentration (EC50) or dose (ED50) of the drug required to produce 50% of that medicament's maximal effect. In certain cases, relative potency may be used, where instead of using units to describe the dose required to achieve a certain endpoint, a ratio of equivalent doses is used. By way of example, medicament A is 10 times more potent than medicament B i.e., it achieves the same effect with 1/10^{th} of the dose.

In certain embodiments, the nucleic acid of the invention, the expression cassette of the invention, the AAV of the invention or the pharmaceutical composition of the invention need to be delivered to a target cell for their use as a medicament. As stated above, ALS, FTD and any atypical presentations mimicking other kinds of neurodegenerative brain diseases primarily affect the brain. Thus, in some embodiments of the invention, the target cell is a central nervous system (CNS) cell. In some embodiments of the invention, the target cell is a neuron. In some specific embodiments of the invention, the target cell is a human frontal brain neuron and/or anterior brain neuron.

In certain embodiments, the nucleic acid of the invention, the expression cassette of the invention, the AAV of the invention or the pharmaceutical composition of the invention are delivered to the spinal cord.

In certain embodiments, the nucleic acid of the invention, the expression cassette of the invention, the AAV of the invention or the pharmaceutical composition of the invention are delivered to the cerebrospinal fluid (CSF).

In certain embodiments, the nucleic acid of the invention, the expression cassette of the invention, the AAV of the invention or the pharmaceutical composition of the invention are delivered to the frontal lobe and/or anterior temporal lobe.

In certain embodiments of the invention, the nucleic acid, the expression cassette of the invention, the AAV of the invention or the pharmaceutical composition of the invention are delivered to the CNS target cell by injection. In certain embodiments, the injection is an intraparenchymal injection. In some embodiments, the injection is an intrathecal injection. In some other embodiments, the injection is a subpial injection. In certain embodiments of the invention, the injections are MRI-guided injections.

In certain embodiments, the nucleic acid, the expression cassette of the invention, AAV of the invention or the pharmaceutical composition of the invention are delivered to the CNS target cell by a combination of delivery methods. By way of a non-limiting example, the combination of delivery methods may comprise intrathecal or subpial injection combined with intracerebroventricular and/or intrastriatal injection; or intrathecal or subpial injection combined with intraparenchymal injections. In some embodiments of the invention, the AAV of the invention or the pharmaceutical composition of the invention are delivered by convection enhanced delivery.

The methods for producing the nucleic acid of the invention comprise any methods for producing nucleic acids, including but not limited to column-based oligonucleotides synthesis and microarray-based synthesizer, all of which would be apparent to the skilled person.

The method for producing the AAV vector of the invention may comprise the steps of: a) culturing a host cell as herein defined above under conditions such that the AAV vector is produced; and, b) optionally, one or more of recovery, purification and formulation of the AAV vector.

The host cell is a host cell that is suitable for the production of AAV vectors. Accordingly, the host cell is a host cell that is amenable to in vitro culture, preferably at large scale. Host cells that are suitable for the production of AAV vectors are well-known in the art and will typically be a mammalian or an insect cell line. Mammalian cell lines for producing AAV vectors are selected from among any mammalian species, including, without limitation, cells such as A549, WEHI, 3T3, 10T1/2, BHK, MDCK, COS 1, COS 7, BSC 1, BSC 40, BMT 10, VERO, WI38, HeLa, a HEK 293 cell (which express functional adenoviral E1), Saos, C2C12, L cells, HT1080, HepG2 and primary fibroblast, hepatocyte and myoblast cells derived from mammals including human, monkey, mouse, rat, rabbit, and hamster. The selection of the mammalian species providing the cells is not a limitation of this invention; nor is the type of mammalian cell, i.e., fibroblast, hepatocyte, tumor cell. Mammalian cell lines for producing AAV vectors in particular include a broad range of HEK293 cell lines, of which the HEK293T cell line is preferred.

Insect cell lines for producing AAV vectors can be any cell line that is suitable for the production of heterologous proteins. Preferably the insect cell allows for replication of baculoviral vectors and can be maintained in culture, more preferably in suspended culture. In a preferred embodiment, the insect cell allows for replication of recombinant parvoviral vectors, including rAAV vectors. For example, the cell line used can be from Spodoptera frugiperda, Drosophila, or mosquito, e.g., Aedes albopictus derived cell lines. Preferred insect cells or cell lines are cells from the insect species which are susceptible to baculovirus infection, including e.g. S2 (CRL-1963, ATCC), Se301, SelZD2109, SeUCR1, Sf9, Sf900+, Sf21, BTI-TN-5B1-4, MG-1, Tn368, HzAm1, Ha2302, Hz2E5, High Five (Invitrogen, CA, USA) and expresSF+® (U.S. Pat. No. 6,103,526; Protein Sciences Corp., CT, USA).

Thus, in some embodiments, the expression cassette or construct is an insect cell-compatible vector or a mammalian cell-compatible vector. An “mammalian cell-compatible vector” is understood to be a nucleic acid molecule capable of productive transformation or transfection of a mammalian cell or cell line. Mammalian cell-compatible vectors are well-known in the art. An “insect cell-compatible vector” is understood to be a nucleic acid molecule capable of productive transformation or transfection of an insect or insect cell. Exemplary insect cell-compatible vectors include plasmids, linear nucleic acid molecules, and recombinant viruses, such as baculoviruses. Any vector can be employed as long as it is insect cell-compatible. The mammalian or insect cell-compatible vector may integrate into the cell's genome but the presence of the vector in the cell need not be permanent and transient episomal vectors are also included. The vectors can be introduced by any means known, for example by chemical treatment of the cells, electroporation, or infection.

The AAV in the supernatant can be recovered and/or purified using suitable techniques which are known to those of skill in the art. For example, monolith columns (e.g., in ion exchange, affinity or IMAC mode), chromatography (e.g., capture chromatography, fixed method chromatography, and expanded bed chromatography), centrifugation, filtration and precipitation, can be used for purification and concentration. These methods may be used alone or in combination. In one embodiment, capture chromatography methods, including column-based or membrane-based systems, are utilized in combination with filtration and precipitation. Suitable precipitation methods, e.g., utilizing polyethylene glycol (PEG) 8000 and NH₃SO₄, can be readily selected by one of skill in the art. Thereafter, the precipitate can be treated with benzonase and purified using suitable techniques. In addition, recovery may preferably comprises the step of affinity-purification of the (virions comprising the) rAAV vector using an anti-AAV antibody, preferably an immobilised antibody. The anti-AAV antibody preferably is a monoclonal antibody. A particularly suitable antibody is a single chain camelid antibody or a fragment thereof as e.g. obtainable from camels or llamas (see e.g. Muyldermans, 2001, Biotechnol. 74:277-302). The antibody for affinity-purification of rAAV preferably is an antibody that specifically binds an epitope on an AAV capsid protein, whereby preferably the epitope is an epitope that is present on capsid protein of more than one AAV serotype. E.g. the antibody may be raised or selected on the basis of specific binding to AAV2 capsid but at the same time also it may also specifically bind to AAV1, AAV3 and AAV5 capsids.

In general, suitable methods for producing an AAV vector according to the invention in mammalian or insect host cells, and means therefore (such as expression constructs for expression of AAV rep proteins), are described, for mammalian cells in: Clark et al. (1995, Hum. Gene Ther. 6, 1329-134), Gao et al. (1998, Hum. Gene Ther. 9, 2353-2362), Inoue and Russell (1998, J. Virol. 72, 7024-7031), Grimm et al. (1998, Hum. Gene Ther. 9, 2745-2760), Xiao et al. (1998, J. Virol. 72, 2224-2232) and Judd et al. (Mol Ther Nucleic Acids. 2012; 1: e54), and for insect cells in: Urabe et al. (2002, Hum. Gene Ther. 13:1935-1943), WO2007/046703, WO2007/148971, WO2009/014445, WO2009/104964, WO2011/122950, WO2013/036118, WO2015/137802, WO2019/016349 and in co-pending applications EP21177449.2, PCT/EP2021/058794 and PCT/EP2021/058798.

The methods for producing the pharmaceutical composition of the invention comprise any methods for producing pharmaceutical compositions, all of which would be apparent to the skilled person. Said methods generally comprise the combination of the nucleic acid or the AAV of the invention with excipients under specific conditions.

Another aspect of the invention relates to a kit comprising the nucleic acid of the invention, the expression cassette of the invention, the AAV vector of the invention, or the pharmaceutical composition of the invention, and an immunosuppressive compound.

In certain embodiments of the invention, the immunosuppressive compound may reduce and/or prevent an immune response induced by administration of the nucleic acid, the AAV vector, or the pharmaceutical composition of the invention.

In another aspect, the invention relates to a cell comprising the nucleic acid of the invention, the expression cassette of the invention, or the AAV of the invention, or a host cell.

In some embodiments, the cell of the invention is a prokaryote cell. In some specific embodiments, the cell of the invention is a bacterial cell. In some embodiments, the cell of the invention is a eukaryote cell. In some embodiments, the cell of the invention is a mammalian cell. In some embodiments, the cell of the invention is an insect cell.

The nucleic acid of the invention or the AAV vector of the invention may be delivered into the cell of the invention by any suitable methods, included but not limited to, transfection, transformation and transduction. For example, the expression cassette or nucleic acid of the invention may be comprised in a plasmid contained in bacteria. The expression cassette or nucleic acid of the invention may also be comprised in a production cell that produces e.g. a viral vector. Said expression cassette may also be provided in a baculovirus vector.

Further detail on host cells comprising an AAV vector according to the invention has been provided elsewhere in this application. Any mammalian cell may be infected by the AAV vector of the invention, including but not limited to a muscle cell, a liver cell, a nerve cell, a glial cell or an epithelial cell. In some preferred embodiments of the invention, the cell to be infected is a human cell.

In another aspect, the present invention relates to a method of treating or preventing a disorder, wherein the method comprises administering the nucleic acid of the invention, the expression cassette, or the AAV of the invention to a subject, thereby treating or preventing the disorder.

In certain embodiments, the disorder is caused by a pathological human c9orf72 gene comprising a GGGGCC hexanucleotide repeat expansion in a non-coding region, wherein the number of GGGGCC hexanucleotide repeats is at least 24.

In certain embodiments, the disorder is ALS, FTD, or any atypical presentation mimicking other kinds of neurodegenerative brain diseases.

In another aspect, the invention relates to the nucleic acid of the invention or the AAVof the invention for use in the manufacture of a medicament for treatment of a disorder.

In certain embodiments, the disorder is caused by a pathological human c9orf72 gene comprising a GGGGCC hexanucleotide repeat expansion in a non-coding region, wherein the number of

GGGGCC hexanucleotide repeats is at least 24. In certain embodiments, the disorder is ALS, FTD or any atypical presentation mimicking other kinds of neurodegenerative brain diseases.

### BRIEF DESCRIPTION OF THE FIGURES

FIG. 1. A schematic representation of chromosome 9 open reading frame 72 (c9orf72) gene sequence (Homo sapiens, NCBI accession number: NG_031977.2) with designed sense miRNA guides (C90).

FIG. 2. A schematic representation of chromosome 9 open reading frame 72 (c9orf72) gene sequence (Homo sapiens, NCBI accession number: NG_031977.2) with designed antisense miRNA guides (C90-AS).

FIG. 3. Homo sapiens pri-miR-451 from miRBase database (www.mirbase.org). Twenty-two nucleotides of the guide strand (underlined) were replaced by the mature C90, C90-AS or C90-SCR.

FIG. 4. Schematic representation of the A) V1S and B) V2S expression cassettes.

FIG. 5. Schematic representation of A) the C90 and C90-AS expression cassettes; the two Luc reporters containing B) c9orf72 sense target sequences comprising exon 1a and part of intron 1 and C) c9orf72 antisense target sequences comprising part of the intron 1 and exon 1a downstream of the Renilla luciferase cassette (RL) and used for in vitro screening of C90 and C90-AS constructs.

FIG. 6. Schematic representation of the expression construct expressing artificial intron 1 c9orf72 mRNA (I-RP99).

FIG. 7. Knockdown efficacy of seventy-nine C90 constructs tested on Luc-C90-sense reporter.

FIG. 8. Knockdown potency of twenty-three C90 constructs in a titration experiment.

FIG. 9. Knockdown efficacy of twenty-four C90-AS constructs tested on Luc-C90-antisense reporter.

FIG. 10. Knockdown potency of seven C90AS constructs in a titration experiment.

FIG. 11. Artificial intronic c9orf72 mRNA knockdown of A) seventy-nine C90 constructs and B) twenty C90 constructs in vitro upon plasmid transfection.

FIG. 12. Total c9orf72 mRNA expression in vitro upon plasmid transfection.

FIG. 13. Long isoform c9orf72 protein expression in vitro upon plasmid transfection.

FIG. 14. Knockdown efficacy of eleven V1S constructs tested on Luc-C90-sense and antisense reporter.

FIG. 15. Knockdown efficacy of twelve V2S constructs tested on Luc-C90-sense and antisense reporter.

FIG. 16. Knockdown potency of three V1S and five V2S constructs in a titration experiment.

FIG. 17. Mature expressed C90 miRNAs determined by NGS. HEK293T were transfected with 50 ng of A) C9026, B) C9046 and C) C9055 construct.

FIG. 18. Abundancy of mature miRNAs in wild type mouse brain (striatum) determined by NGS. Mice were injected in the striatum with 1.5E+11 gc/hemisphere (n=2) AAV5-C9026, AAV5-C9046 or AAV5-C9055.

FIG. 19. Processing of mature miRNAs in mouse brain (striatum) determined by NGS. Mice were injected in the striatum with 1.5E+11 gc/hemisphere (n=2) AAV5-C9026, AAV5-C9046 or AAV5-C9055.

FIG. 20. Vector DNA copies determined by QPCR in striatum. BAC-C9-112 mice were injected in the striatum with 3E+10 gc/hemisphere (n=5) AAV5-C90-SCR1, AAV5-C9026 or AAV5-C9046. Wild type and transgenic mice were dosed with vehicle.

FIG. 21. Intronic C9orf72 mRNA expression determined by RT-QPCR in striatum in BAC-C9-112. Statistics: one-way ANOVA with Dunnett's post-hoc test.

FIG. 22. C9orf72 mRNA expression was determined by RT-QPCR in striatum of BAC-C9-112 mice. A) Total human C9orf72 mRNA and B) human variant 2 C9orf72 mRNA expression.

FIG. 23. Body weights of transgenic BAC-C9-112 mice injected with AAV5-C90-SCR1, AAV5-C9026 or AAV5-C9046 during the in-life phase of A) male mice and B) female mice.

FIG. 24. Mature miRNA expression determined by RT-QPCR in striatum of BAC-C9-112 mice.

FIG. 25. Vector DNA copies determined by QPCR in A) striatum and B) rostral cortex. AAV9-C9orf72 (G₄C₂)₁₄₉ repeats-induced mice were injected in the striatum with 3E+10 or 1.5E+11 gc/hemisphere (n=6) AAV5-C90-SCR1 (only high dose), AAV5-C9046 or AAV5-C9055. Wild type and AAV9-C9orf72 (G4C2) 149 repeats-induced mice were dosed with vehicle.

FIG. 26. Intronic C9orf72 mRNA expression determined by RT-QPCR in A) striatum and B) rostral cortex of AAV9-C9orf72 (G₄C₂)₁₄₉ repeats-induced mice.

FIG. 27. Body weights of treated AAV9-(G₄C₂)₁₄₉-induced mice during the in-life phase.

FIG. 28. Mature miRNA expression determined by RT-QPCR in striatum in AAV9-C9orf72 (G₄C₂)₁₄₉ repeats-induced mice.

FIG. 29. C9orf72 mRNA expression in ALS motor neurons (n=3) upon transduction with AAV6-C9046 and AAV6-C90-SCR1 obtained with A) RT-PCR experiment and B) RNA-sequencing of ALS and control motor neurons and C) RNA-sequencing of ALS motor neurons.

FIG. 30. Volcano plots visualizing the significantly differentiated expressed genes in A) ALS motor neurons transduced with AAV6-C9046 vs AAV6-C90-SCR1; B) control motor neurons transduced with AAV6-C9046 vs AAV6-C90-SCR1 and C) between untraduced ALS and control motor neurons.

FIG. 31. Vector DNA copies determined by QPCR in A) striatum and B) rostral cortex in mice sacrificed at 18 weeks post-injection. AAV9-C9orf72 (G4C2)-149 repeats mice were injected in the striatum with 3E+10 or 1.5E+11 gc/hemisphere (n=6) with AAV5-C90-SCR1 (only high dose), AAV5-C9046 or AAV5-C9055. Control wild type and AAV9-C9orf72 (G4C2)-149 repeats mice were dosed with vehicle.

FIG. 32. Intronic C9orf72 mRNA expression determined by RT-QPCR in A) striatum and B) rostral cortex of AAV9-C9orf72 (G4C2)-149 repeats mice sacrificed at 18 weeks post-injection.

FIG. 33. Mature miRNA expression determined by RT-QPCR in striatum and rostral cortex in AAV9-C9orf72 (G4C2)-149 repeats mice at 18 weeks post-injection.

FIG. 34. Mature C9046 expression determined by TaqMan RT-QPCR in striatum and rostral cortex in AAV9-C9orf72 (G4C2)-149 repeats mice at 18 weeks post-injection.

FIG. 35. Mature C9046 expression determined by TaqMan RT-QPCR in striatum and rostral cortex in AAV9-C9orf72 (G4C2)-149 repeats mice at 8 weeks post-injection and transgenic BAC-C9-112 mice.

FIG. 36. Results of MSD assay for A) poly (GP) and B) poly (GA) quantification in cortex of AAV9-C9orf72 (G4C2)-149 repeats mice at 18 weeks post-injection.

FIG. 37. Immunohistochemistry results for poly (GA) aggregates in AAV9-C9orf72 (G4C2)-149 repeats mice at 18 weeks post-injection. AAV9 induced controls show round, perinuclear poly (GA) aggregates (indicated by arrows), treated mice show different pattern which is a diffused poly (GA) staining, which filled the entire cytoplasm (indicated by arrowheads).

FIG. 38. Chromogenic in situ hybridization results for sense RNA foci in striatum of AAV9-C9orf72 (G4C2)-149 repeats mice at 18 weeks post-injection with A) representing the stained area ration; B) the average aggregate size and C) the number of RNA foci per mm².

FIG. 39. Chromogenic in situ hybridization results for antisense RNA foci in striatum of AAV9-C9orf72 (G4C2)-149 repeats mice at 18 weeks post-injection with A) representing the stained area ration; B) the average aggregate size and C) the number of RNA foci per mm².

FIG. 40. Results of the contextual learning in fear conditioning test in C9orf72 (G4C2)-149 repeats-mice at 18 weeks post-injection.

FIG. 41. Results of the wire hang test in C9orf72 (G4C2)-149 repeats-mice at 18 weeks post-injection.

FIG. 42. Overview of body weights at 25 weeks in life of C9orf72 (G4C2)-149 repeats-mice.

### EXAMPLES

### Materials and Methods

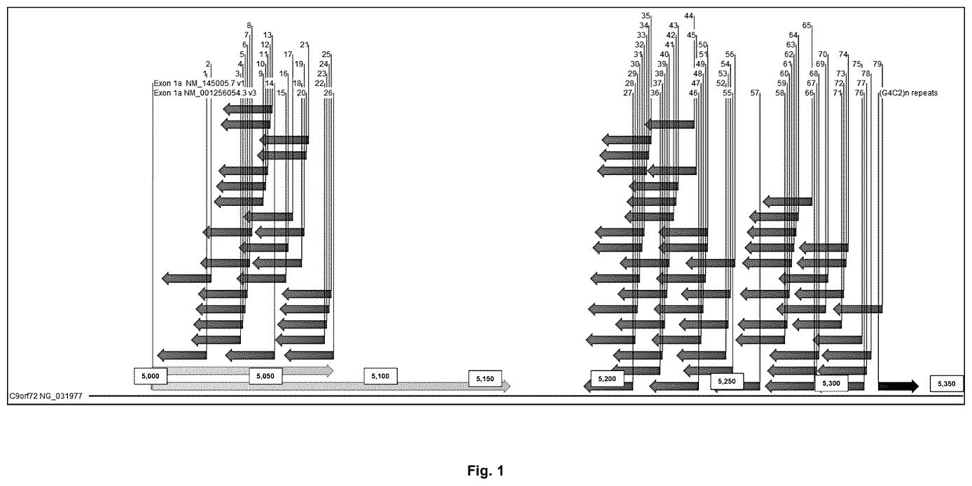

C9orf72 miRNA guide strand design. The sense C9Os (miRNA sense guide strands) were designed to target common RNA sequences of the C9orf72 variant 1 and variant 3 for a selective silencing approach of the transcripts containing the pathological G4C2 repeat expansion (FIG. 1). The target regions of the C9orf72 mRNA sequences are a portion of the exon 1a present in both, C9orf72 variant 1 mRNA (Homo sapiens, NCBI accession number NM_145005.7: position 1-80 nucleotides [nts], SEQ ID NO. 1) and variant 3 mRNA (Homo sapiens, NCBI accession number NM_001256054.3: position 1-80 nts, SEQ ID NO. 2); and a region of the intron 1 C9orf72 gene that is retained when the pathological repeat expansion is present (Homo sapiens, NCBI accession number NG_031977.2:5159-5322 nts, SEQ ID NO. 3). Each of the conserved sequences was used to generate a number of different guide strands having 22 nts. Twenty-six guides targeting sense C9orf72 exon 1a RNA (named C901-C9025,SEQ ID NOs. 4-20 corresponding to candidates that showed >70% of Luciferase knockdown and SEQ ID NOs. 135-143) and fifty-three guides targeting sense C9orf72 intron 1 RNA (named C9027-C9079, SEQ ID NOs. 21-61 corresponding to candidates that showed >70% of Luciferase knockdown and SEQ ID NOs. 144-155) were tested in vitro.

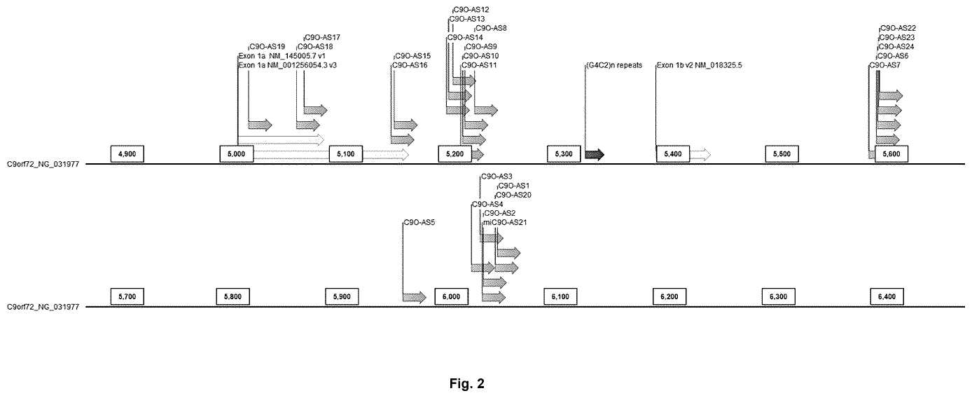

The antisense C9Os encode guide strands to target antisense RNA sequences derived from the bidirectional transcription of the C9orf72 gene for a selective silencing approach of the antisense transcripts containing the pathological G4C2 repeat expansion (FIG. 2). The target regions of the antisense C9orf72 mRNA sequences were identified through literature searches and analysis of RNAseq data and are present in the exon 1a and intron 1 (Homo sapiens, NCBI accession number NG_031977.2:5001-6071 nts SEQ ID NO. 163). The antisense C9orf72 RNA region was used to generate a number of different guide strands having 22 nts (a total number of 59 guides for exon 1a and 141 guides for intron 1). Three guides targeting antisense C9orf72 exon 1a RNA (named C90-AS17-AS19, SEQ ID NOs. 62-64) and two guides spanning antisense C9orf72 exon 1a and intron 1 RNA (named C90-AS15 and C90-AS16, SEQ ID NO. 65 corresponding to candidate that showed >80% of Luciferase knockdown and SEQ ID NOs. 164) were selected for in vitro testing. Nineteen guides targeting antisense C9orf72 intron 1 RNA (named C90-AS1—AS14, C90-AS20—AS24, SEQ ID NOs. 66-68 corresponding to candidates that showed >80% of Luciferase knockdown and SEQ ID NOs. 165-180) were tested in vitro.

The antisense C9Os encode antisense guide strands to target antisense RNA sequences derived from the bidirectional transcription of the C9orf72 gene for a selective silencing approach of the antisense transcripts containing the pathological G4C2 repeat expansion (FIG. 2). The target regions of the antisense C9orf72 mRNA sequences were identified through literature searches and analysis of RNAseq data and are present in the exon 1a and intron 1 (Homo sapiens, NCBI accession number NG_031977.2:5001-6071 nts SEQ ID NO. 163). The antisense C9orf72 RNA region was used to generate a number of different guide strands having 22 nts (a total number of 59 guides for exon 1a and 141 guides for intron 1). Three guides targeting antisense C9orf72 exon 1a RNA (named C90-AS17-AS19, SEQ ID NOs. 62-64) and two guides spanning antisense C9orf72 exon 1a and intron 1 RNA (named C90-AS15 and C90-AS16, SEQ ID NO. 65 corresponding to candidate that showed >80% of Luciferase knockdown and SEQ ID NOs. 164) were selected for in vitro testing. Nineteen guides targeting antisense C9orf72 intron 1 RNA (named C90-AS1-AS14, C90-AS20-AS24, SEQ ID NOs. 66-68 corresponding to candidates that showed >80% of Luciferase knockdown and SEQ ID NOs. 165-180) were tested in vitro. The selected C90-AS guides meet the following criteria: conservation with monkey c9orf72 gene sequence (Macaca mulatta, NCBI accession number NC_041768.1), the miRNA guide sequence should not include a stretch of >4 G or >4 C nt, a GC content between 20% and 70%, a GC seed content between 40% and 70%, pre-miRNA sequence folding energy between −45 and −55 kcal/mole and no matching with endogenous miRNA seeds.

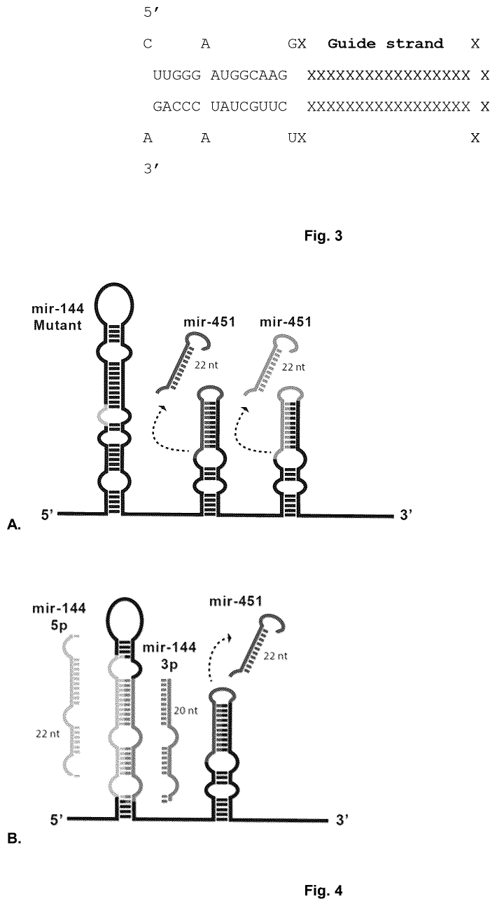

Guide sequences were incorporated into human pri-miRNA miR-451 scaffold sequences and the mFold program (http://unafold.rna.albany.edu/?q=mfold) was used with standard settings to determine whether the candidates are folded into the secondary structures as depicted in FIG. 3.A. If not folded into the predicted secondary structure, the sequence was adapted, which did not involve adapting the first RNA sequences, such that the correct structure was folded by the program.

The C9Os scaffolds that combine sense and antisense miRNA guide strands were generated using the most potent C90 sense candidates C9026, namely C9046 and C9055, together with the most potent antisense guide strand, C90-AS20. Scrambled control constructs (scrambled sequences SEQ ID NO. 157 to 161) were generated by combining the ALS001 (SEQ ID NO. 162) and the C90-SCR-1 (SEQ ID NO. 156). The version 1 scaffold (V1S or V2S) comprises the mutated miR-144 helper hairpin (SEQ ID NO. 181) combined with two miR-451 downstream scaffolds (SEQ ID NOs. 182-192). In this design the miR-451 scaffolds contain the C9orf72 sense—as well as antisense targeting guide sequences (FIG. 4.A). The effect of positioning of the mir-451 sense and antisense candidate scaffolds with respect to the miR-144 helper hairpin, (miR-144 proximal or distal positioning) was previously tested; with this information, eleven V1S constructs were designed that target the C9orf72 sense and antisense repeat expanded mRNAs (V1S-13-V1S-23, SEQ ID NOs. 182-192).

The version 2 scaffold (V2S) comprises the miR-144 hairpin (expressing targeting miRNA-either as passenger-guide or guide-guide, as explained below) combined with one mir-451 downstream scaffold (FIG. 4.B). In this design the miR-144 scaffold contains the C9orf72 sense targeting guide the miR-451 scaffold contains antisense targeting guide sequences. Placement of the C9orf72 sense targeting guides C9026, C9046 and C9055 was either on the 5p arm (=“guide”-guide) or 3p arm (=“passenger”-guide) of miR-144. Additionally, point mutations were introduced in the 3p or 5p arm at specific positions. Twelve V2S constructs were generated to target the C9orf72 sense and antisense repeat expanded mRNAs (V2S-42-V2S-53, SEQ ID NOs. 193-204).

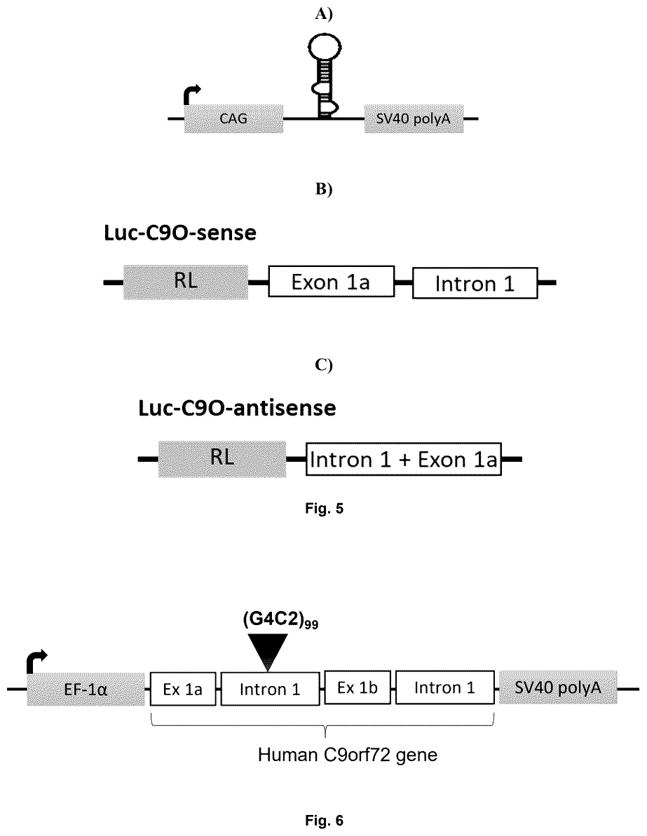

DNA constructs. The C90 sense and antisense, the V1S and V2S constructs and the scrambled controls were embedded in the human pre-miR-451 scaffold, flanked by 206 nts of 5′ and 205 nts 3′ flanking regions, Nhel and BamHI restriction sites were added respectively at the 5′ and 3′ and the complete sequence was gene synthesized (GeneWiz, Azenta Life Sciences). The pri-C90 cassettes were expressed from the CMV immediate-early enhancer fused to chicken β-actin promoter (CAG promoter, SEQ ID NO. 205) and terminated by the simian virus 40 polyadenylation (SV40 polyA, SEQ ID NO. 206) signal (FIG. 5.A), while the pri-V1S/V2S cassettes were expressed from a hybrid form of the CBA promoter (P1; SEQ ID NO. 207). Two luciferase reporters Luc-C90-sense (SEQ ID NO. 208) and Luc-C90-antisense (SEQ ID NO. 209) were generated by respectively combining the fragments of the exon 1a and intron 1 sense (Homo sapiens, the NCBI accession number NM_001256054.3: position 1-158 nts, and the NCBI accession number NG_031977.2: positions 5159-5320 nts, FIG. 5.B), and exon 1a and intron 1 antisense (Homo sapiens, the NCBI accession number NG_031977.2: position 5001-5277 nts and 5542-5591 nts; FIG. 5.C). Flanking regions at the 5′ and 3′ were included with Xhol and Notl restriction sites. The synthesis of the sequences as well as the cloning into the 3′UTR of the Renilla luciferase (RL) gene of the psiCHECK-2 vector (Promega, Thermo Fisher Scientific) were performed by GeneArt for Luc-C90-sense (Thermo Fisher Scientific) and LucC90-antisense GeneWiz (Azenta Life Sciences).

An expression vector encoding for the intronic human c9orf72 mRNA harbouring 99 G4C2 repeats (I-RP99) was generated. The Human elongation Factor-1 alpha (EF-1a) promoter (SEQ ID NO. 210) was used to express C90 target sequences (NG_031977.2: position 5001-6340 nts and 11683-11702 nts; SEQ ID NO. 211; GeneArt, Thermo Fisher Scientific). The flanking regions of the insert at the 5′ and 3′ contained Nhel and Notl restriction sites. Upon successful subcloning of the intronic c9orf72 insert, synthetic DNA oligonucleotides (GGGGCC) 20 and (CCCCGG) 20 were used to subclone >90 sense (GGGGCC) repeats by Bbsl digestion of both entry plasmids and DNA oligonucleotides. Nanopore sequencing was performed to characterize the exact number of inserted repeats (KeyGene N.V.) and the results showed that all the generated clones harboured >90 sense repeats (FIG. 6).

Transfection assays and cells. The human embryonic kidney 293 cells containing the SV40 T-antigen (HEK239T) cells were maintained in Dulbecco's modified Eagle's medium (Thermo Fisher Scientific) containing 10% of heat inactivated fetal bovine serum (Thermo Fisher Scientific) at 37° C. and 5% CO2. For luciferase assays and endogenous lowering of mRNA from artificial intronic c9orf72 constructs having 90 repeats, cells were seeded in 24-well plates at a density of 1E+05 cells per well in Dulbecco's modified Eagle's medium (Thermo Fisher Scientific) one day prior transfection. Transfections were performed with Lipofectamine 2000 reagent (Thermo Fisher Scientific) according to the manufacturer's instructions.

Dual Reporter Luciferase Assay. HEK293T cells were cotransfected in triplicate with sense C90, antisense C90 or V1S/V2S expression constructs and luciferase reporters that contain both the RL gene fused to C9orf72 target sequences and the Firefly luciferase (FL) gene. pBluescript was added to transfect equal amounts of DNA. Transfected cells were assayed at 48 hours post-transfection in 100 μl 1× passive lysis buffer (Promega, Thermo Fisher Scientific) by gentle rocking for 15 minutes at room temperature. The cell lysates were centrifuged for 5 minutes at 4,000 rpm and 10 μl of the supernatant was used to measure FL and RL activities with the Dual-Luciferase Reporter Assay System (Promega, Thermo Fisher Scientific). Relative luciferase activity was calculated as the ratio between RL and FL activities.

Measurement of repeat-containing C9orf72 intron 1 mRNA knockdown expressed in HEK293T. RT-QPCR was performed to confirm miRNA expression by knockdown of mRNA from an artificial intronic C9orf72 constructs having 99 repeats. HEK293T cells were cotransfected in duplicate with all the sense C90 constructs and the artificial intronic C9orf72 constructs having 90 repeats. Cell monolayers were harvested with lysis buffer from MagMAX™ mirVana™ Total RNA Isolation Kit

(Thermo Fisher Scientific) 48 hours after transfection and RNA was isolated using MagMAX™ mirVana™ Total RNA Isolation Kit (Thermo Fisher Scientific) according to manufacturer's instructions. DNase treatment was performed by using TURBO DNAse™ provided in the RNA isolation kit (Thermo Fisher Scientific), for cDNA synthesis Maxima First Strand cDNA Synthesis Kit (Thermo Fisher Scientific) was used according to manufacturer's instructions. QPCR was performed with TaqMan ready-to-use primer-probe (Thermo Fisher Scientific) from Gene Expression Assay (Thermo Fisher Scientific) for B-actin (ACTB) as housekeeping gene (Assay ID: Hs01060665_g1, Thermo Fisher Scientific). TaqMan QPCR assays was developed to measure intronic C9orf72: forward primer SEQ ID NO. 212; reverse primer SEQ ID NO. 213; and probe SEQ ID NO. 214. Relative gene expression data were obtained normalizing intronic C9orf72 data with human ACTB as reference gene (SEQ ID NO. 215 and 216). Results are shown relative to the C90-SCR1 sample that is set to 100% Luciferase expression.

Measurement of total c9orf72 protein in HEK293T cells. HEK293T cells have been shown to have detectable protein levels in Western blot of the short (24 kDa, encoded by V1 C9orf72 mRNA) and of the long C9orf72 protein isoform (54 kDa encoded by V2 and V3 C9orf72 mRNAs. Therefore, lysates of 48 hrs post-transfected HEK293T with 250 ng of selected C9Os were used to quantify V2-V3 proteins. The C9orf72 antibody #ab221137 (Abcam) that can detect all C9orf72 isoforms V1 (27 kDa) and V2/3 (51 kDa) was used to measure total C9orf72 protein expression. As reference protein for normalization ACTB was used (#8226, Abcam).

RNA isolation and next-generation sequencing (NGS). HEK293T cells were transfected with 50 ng of selected C9Os constructs using Lipofectamine 2000 reagent (Thermo Fisher Scientific) and total RNA was isolated from cells 48 hours post-transfection using TRIzol® Reagent (Thermo Fisher Scientific) and Direct-zol RNA Miniprep (Zymo Research,) according to the manufacturer's protocol. RNA samples were treated with dsDNase from Thermo Fisher Scientific according to manufacturer's instructions. For sequencing, total RNA samples were sent out for small RNA sequencing (GenomeScan B.V.). Small RNA sequencing libraries for the Illumina platform were prepared and sequenced at GenomeScan B.V.

NGS data analysis. Analysis of the miRNA expression and processing in transfected HEK293T cells was performed using CLC Genomics Workbench 10. Trimming and concatenation of the reads was performed by GenomeScan B.V. Next, the obtained unique small RNA reads were annotated using miRNA human database (miRBase) and aligned to the reference sequences of the pri-C90 constructs. The percentage of expression of C90 in the total pool of endogenous miRNAs was calculated by the software CLC Genomics Workbench 10 during the annotation process. To investigate the processing of C9Os, length and percentage of each mature miRNA species were assessed by considering the top 20 most abundant annotations (set to 100%) against the appropriate pri-C90 sequence (SED ID. NO. 217-219).

DNA constructs for Baculovirus seed generation and AAV5 production in HEK293T cells. The expression cassettes were incorporated in a plasmid encoding the AAV ITRs. The expression cassettes comprising a promoter sequence driving the expression of miRNA targeting mutant sense C9orf72 and/or antisense C9orf72. Expression cassettes used in the examples comprise e.g. promoter sequences such as listed in SEQ ID NO. 205 and 207 representing the CAG promoter or the P1 promoter, combined with miRNA encoding sequences such as listed e.g. in SEQ ID NO. 151-153 (pri-C90). Exemplary expression cassettes as used in the studies being listed in SEQ ID NO. 220 and 221 (CAG-pri-C90 and P1-pri-V1S/V2S). The cloning was performed by GeneWiz (Azenta Life Sciences).

AAV vectors. Recombinant AAV5 vectors were produced by PEI transfection of HEK29T cell with two plasmids encoding for Rep-Cap and the Transgene (Sirion Biotech). Following two step purification with primary capture with POROS™ CaptureSelect™ AAV-X resin (Thermo Fisher Scientific) and iodixanol gradient the titer of the purified AAV was determined using QPCR.

Furthermore, recombinant AAV5 and AAV9 particles were produced by infecting serum-free SF+insect cells (Protein Sciences Corporation, Meriden, Connecticut, USA) with two Baculoviruses, one encoding Rep/Cap combination, with the second carrying a transgene construct. Following standard protein purification procedures on a fast protein liquid chromatography system (AKTA Avant 150, GE 30 Healthcare) using AVB sepharose (GE Healthcare) the titer of the purified AAV was determined using QPCR. Recombinant AAV6 particles were produced by infecting serum-free SF+insect cells (Protein Sciences Corporation, Meriden, Connecticut, USA) with three Baculoviruses encoding for Rep, Cap and transgene and incubated for 72 hours. AAV6 particles were purified from the crude lysate using AVB Sepharose™ High Performance (GE Healthcare), eluted using a 0.2 M glycine/HCl buffer at pH 2.5 and neutralized using a 0.5 M tris/HCl buffer at pH 8.5. The purified recombinant AAV6 particles were aliquoted and stored at −80° C. until further use. The titer of the purified AAV was determined using QPCR with primers SEQ. ID 261 and SEQ. ID 262 and probe SEQ. ID 263.

AAV5 transduction of HEK293T cells. HEK293T cells were maintained in Dulbecco's modified Eagle's medium (Thermo Fisher Scientific) containing heat inactivated fetal bovine serum (Thermo Fisher Scientific), at 37° C. and 5% CO2. For transduction assays, cells were seeded in 24-well plates at a density of 1E+05 cells per well in Dulbecco's modified Eagle's medium (Thermo Fisher Scientific) one day prior transduction. Cells were transduced with 100 μL of AAV5 vectors at a multiplicity of infection (MOI) of 1E+04, 1E+05 and 1E+06 genome copies (gc) per cell in triplicate. Two days post-transduction, the monolayers were harvested in 100 μl RLT plus buffer (AllPrep DNA/RNA Micro Kit, Qiagen). Three wells belonging to the same condition were pooled for DNA extraction.

Vector DNA isolation and quantification from cells. DNA extraction was performed using AllPrep DNA/RNA Micro Kit (Qiagen) following manufacturer's instructions. Vector genome copies were quantified by using TaqMan QPCR assay (Thermo Fisher scientific) (SEQ ID NO. 222-224) and ACTB SybrGreen assay was used as loading control gene.

AAV6 transduction of human induced pluripotent stem cell (hiPSC)-derived motor neurons. Healthy control (Donor #: NDS00159; Catalog #: ND41865) and ALS-C9orf72 (Donor #: NDS00269; Catalog #: ND50075) hiPSC were purchased from the National Institute of Neurological Disorders and Stroke. The cells were differentiated in spinal cord motor neurons following the protocol of Du and collaborators (2015) with minor modifications. Three independent transduction experiments were performed on mature control and ALS motor neurons (differentiation day 35). Briefly, single-cells were seeded into 24-well PDL & laminin pre-coated plates at 1.68E+05 cells/cm² at differentiation day 19, this was followed up by two weeks of maturation, after which the cultures were infected with AAV6-C9046 (MOI of 1.00E+07 GC/cell), AAV6-C90-SCR1 (MOI of 1.00E+07 GC/cell) or phosphate-buffered saline (Sirion Biotech GmbH). Monolayers were harvested 5 days post-transduction. Total genomic DNA and RNA fractions were collected by using an AllPrep DNA/RNA Mini Kit (Qiagen; Cat #: 80204). RNA integrity was checked on the Agilent TapeStation 4200 (RIN>7 was required for following RNA-sequencing analysis).

RNA-sequencing data analysis of AAV6-transduced hiPSC-derived motor neurons. Total RNA sequencing was performed at GenomeScan B.V. using Illumina NovaSeq6000 sequencing, 30 million paired-end reads. Data analysis was performed with CLC Genomics Workbench (v22.0.2). Briefly, levels of C9orf72 gene expression were retrieved by mapping the reads on the human reference genome (Genome assembly: GRCh38.p14) and C9orf72 mRNA variants V1, V2 and V3 were shown as target gene transcript reads per million reads (TPM). To investigate potential differential expressed genes (DEGs) due to C9046 expression differential expression analysis and GO enrichment analysis were performed in CLC Genomics Workbench. Significantly DEGs were determined using a statistical threshold corrected for multiple testing using the false discovery rate (FDR) adjustment (FDR-adjusted P≤0.05; fold change≥2). Additionally, the web-tool Panther (v17.0) and GOrilla (Eden et al., 2009) were also used to identify and visualize enriched GO terms of a list of genes.

### RNA Isolation and Next-Generation Sequencing (NGS) Data Analysis of Mouse Samples

Total RNA was isolated from striatum samples of wild type mouse injected with AAV5-miC9026, AAV5-miC9046 and AAV5-miC9055 using TRIzol® Reagent (Thermo Fisher Scientific) and Direct-zol RNA Miniprep (Zymo Research,) according to the manufacturer's protocol. For sequencing, total RNA samples were sent out for small RNA sequencing. At GenomeScan B.V. small RNA sequencing libraries for the Illumina platform were prepared using the Nextflex Small RNA-seq kit and sequencing was performed. Analysis of the miRNA expression and processing was performed using CLC Genomics Workbench 21. Trimming and concatenation of the reads was performed by GenomeScan B.V. Next, the obtained unique small RNA reads were checked for trimming, annotated using miRNA human database (miRBase) and aligned to the reference sequences of the pri-C90 constructs with CLC Genomics Workbench 21. The percentage of expression of C90 in the total pool of endogenous miRNAs was calculated by the software CLC Genomics Workbench 21 during the annotation process. To investigate the processing of C9Os, length and percentage of each mature miRNA species were assessed by considering all the annotated forms (set to 100%) against the appropriate pri-C90 sequence (SED ID. NO. 217-219).

Purification of DNA and Total RNA from Mouse Striata and Cortex

Snap-frozen mouse striatum and cortex tissue samples were crushed using CryoPrep System (Covaris) and tissueTube TT1 Extra Thick or tissueTUBE TT05M (Covaris), depending on the size of the tissue. Powdered tissue was homogenized using Lysis Matrix D tubes (MP Biomedicals), after homogenization, RNA and DNA were isolated using AllPrep DNA/RNA 96 kit (Qiagen) according to manufacturer's instructions. Total amount of DNA and RNA were quantified using NanoPhotometer® N120 (Implen) and the purity was evaluated by measuring the OD at 230, 260 and 280 nm.

cDNA Synthesis and Quantitative Real-Time PCR (QPCR)

After extraction, RNA samples were first treated with dsDNase provided in the cDNA synthesis Maxima First Strand cDNA Synthesis Kit (Thermo Fisher Scientific) in order to remove any potential DNA contamination carried over during RNA extraction. Afterwards, cDNA was synthesized with cDNA synthesis Maxima First Strand cDNA Synthesis Kit (Thermo Fisher Scientific) according to manufacturer's protocol. RT-QPCR was performed to quantify intronic human C9orf72 mRNA lowering in mouse in vivo samples. TaqMan chemistry was used and primers SEQ ID NO. 239 and SEQ ID NO. 240, and probe SEQ ID NO. 241 (Table 3). Absolute quantification of C9orf72 mRNA molecules per ug of RNA was performed using a standard line (1E+8-12.5 genome copies/reaction). To quantify variant 2 (V2) and total human C9orf72 mRNA molecules TaqMan QPCR assays were developed. V2 C9orf72 mRNA expression was quantified using primers SEQ ID NO. 242 and SEQ ID NO. 243, and probe SEQ ID NO. 244 (Table 3). QPCR of the reference genes was performed with TaqMan ready-to-use primer-probe (Thermo Fisher Scientific) from Gene Expression Assay (Thermo Fisher Scientific) for Glucuronidase Beta (GUSB, Assay ID: mm01197698_m1, Thermo Fisher Scientific) and for Hypoxanthine Phosphoribosyltransferase 1 (HPRT, Assay ID: mm03024075_m1, Thermo Fisher Scientific). Relative gene expression data were obtained normalizing intronic C9orf72 data with mouse GUSB and HPRT as reference genes (ddCt method). Results are shown relative to the C90-SCR1 sample that is set to 100%. miRNA expression was quantified by modifying the method described by Cirera et al. (2014 Methods Mol Biol. 1182:73-81), cDNA was generated using Maxima reaction mix and enzyme mix from Maxima First Strand cDNA synthesis kit (Thermo Fisher Scientific), ATP/poly (A) polymerase (Bioke) and the reverse transcription primer SEQ ID NO.245 (Table 3). Multiple SYBRgreen assays were designed for each miC90 to specifically amplify 22 nucleotides, 23 nucleotides and 24 nucleotides expressed miRNA isoforms. After testing the assays with the appropriate RNA oligos, the assay showing the best amplification of the standard line (slope between −3.1 and −3.5 and R₂≥0.990) were selected: for miC90-SCR1 the 23 nucleotides assay [primers SEQ ID NO. 246 and SEQ ID NO. 247; (Table 3)], for miC90-26 the 22 nucleotides assay [primers SEQ ID NO. 248 and SEQ ID NO. 249; (Table 3)], for miC90-46 the 23 nucleotides assay [primers SEQ ID NO. 250 and SEQ ID NO. 251; (Table 3)] and for miC90-55 the 22 nucleotide assay [primers SEQ ID NO. 252 and SEQ ID NO. 253; (Table 3)] Absolute quantification of miRNA molecules per ug of RNA was performed by using standard lines created for each miRNA assay using artificial RNA oligo [miC90-SCR1 RNA oligo SEQ ID. NO. 254; miC90-26 RNA oligo SEQ ID. NO 255, miC90-46 RNA oligo SEQ ID. NO 256, miC90-55 RNA oligo SEQ ID. NO. 257; (Table 3)]. AAV5 vector DNA QPCR was performed using TaqMan chemistry and primers SEQ ID NO. 258 and SEQ ID NO. 259 and probes SEQ ID NO. 260. To calculate the genome copies per ug of DNA a standard line was used (1E+8-12.5 genome copies/reaction).

TABLE 3
Primes, probes and artificial RNA oligos.

| SEQ ID NO. | ID | SEQUENCE |
|---|---|---|
| 239 | Primer | AGGGTGAACAAGAAAAGACCTG |
| 240 | Primer | CGGTTGTTTCCCTCCTTGTTTT |
| 241 | Probe | TAAAGATTAACCAGAAG |
| 242 | Primer | CGG TGG CGA GTG GAT ATC TC |
| 243 | Primer | TGG GCA AAG AGT CGA CAT CA |
| 244 | Primer | TAA TGT GAC AGT TGG AAT GC |
| 245 | Primer | CAGGTCCAGTTTTTT TTTTTTVN |
| 246 | Primer | AGGCGACTTGTAACGTCA |
| 247 | Primer | CAGTTTTTT TTTTTACGCCTCT |
| 248 | Primer | GCAAGCGTCATCTTTTACG |
| 249 | Primer | GTCCAGTTTTTT TTTTTTCCCA |
| 250 | Primer | CAGTCAGGTCTTTTCTTGTTCA |
| 251 | Primer | CCAGTTTTT TTTTTCGGGT |
| 252 | Primer | CGCAGTCTGGTTAATCTTTATCAG |
| 253 | Primer | GGTCCAGTTTTTT TTTTTTGAC |
| 254 | Artificial RNA oligo | GCGACUUGUAACGUCAGAGGCGU |
| 255 | Artificial RNA oligo | CAAGCGUCAUCUUUUACGUGGG |
| 256 | Artificial RNA oligo | UCAGGUCUUUUCUUGUUCACCCG |
| 257 | Artificial RNA oligo | UCUGGUUAAUCUUUAUCAGGUC |
| 258 | Primer | AATGATTAACCCGCCATGCT |
| 259 | Primer | GGGAGGTGTGGGAGGTTT |
| 260 | Probe | ACTTATCTACAGATCTGCGGCCGCT |
| 261 | Primer | GGCGTACTTGGCATATGATACA |
| 262 | Primer | AGTAACGCCAATAGGGACTTTC |
| 263 | Probe | TTACGGTAAACTGCCCACTTGGCA |

Quantification of mature C9046 with TagMan RT-QPCR. RNA was extracted from mouse brains as previously described and reverse transcribed using the TaqMan MicroRNA Reverse Transcription kit (Thermo Fisher Scientific, Cat #4366597) and a custom stem-loop primer specific for miC9046 (26 nts). Custom TaqMan QPCR small RNA assays (Thermo Fischer Scientific, Cat #4398988, assay ID: CTTZ9XY) in combination with TaqMan Fast Universal kit (Thermo Fisher Scientific, Cat #4352042) were used to measure the most abundant miC9046 species. A serial dilution of the 26 nucleotides synthetic RNA (Integrated DNA Technologies) was used to calculate the number of miC9046 molecules/ug total RNA in each sample.

In silico assessment of potential off-target transcripts of C9046 miRNA guide. In silico off-target prediction was performed using Bowtie (version 1.3.1), an ultrafast, memory-efficient short read aligner (Langmead et al., 2009). For each guide RNA, guide nucleotides 2 through 17 (g2-g17) and guide nucleotides 2 through 15 (g2-g15) were aligned to the human reference transcriptome allowing for a single mismatch. Output from this analysis was further annotated using the R/Bioconductor package biomaRt (Durinck et al., 2009).

Poly-Glycine-Proline (GP) quantification with Meso Scale Discovery (MSD) assays. Poly (GP) level was quantified by The Jackson Laboratory in total cortex lysates of 18 weeks post-injection sacrificed mice by using a proprietary MSD assay. In addition, poly (GP) level was quantified in house in rostral cortex tissue lysates of 8 weeks or 18 weeks post-injection sacrificed mice. Samples were lysed in radioimmunoprecipitation assay buffer (RIPA) buffer and measured using an anti-Poly (GP) antibody (Merck, ABN1358) in a sandwich immunoassay based on MSD platform. The plates were read on a MESO QuickPlex SQ 120 MM instrument.

Poly-Glycine-Alanine (GA) with MSD assay. Poly (GA) level was quantified in house in rostral cortex tissue lysates of 8 weeks or 18 weeks post-injection mice. Samples were lysed in RIPA buffer and sonicated just before use. They were then measured using an anti-Poly (GA) antibody (Merck, MABN889) in a sandwich immunoassay based on MSD platform. The plates were read on a MESO QuickPlex SQ 120 MM instrument.

Analysis of repeat dipeptide proteins (DPRs) aggregates. RNA foci sense and antisense. A subset (n=3-6) of the AAV9-C9orf72 (G4C2)-149 repeats mice sacrificed at 18 weeks post-AAV5-injection were dedicated to analysis of ALS pathological markers. Briefly, at The Jackson Laboratory facility (Bar Harbor, ME) following saline perfusion, animals dedicated for histology were transcardially perfused with 10 mL ice-cold 4% PFA in saline and the whole brain, spinal cord cervical, thoracic and lumbar segments were collected. Samples were post-fixed in cold 4% PFA (tissue: fixative ratio 1:20) in saline for 48 h at 4° C. Samples were embedded in the paraffin with the midsagittal plane facing down and kept at JAX for DPRs immunohistochemistry (IHC) and RNA foci sense and antisense chromogenic in situ hybridization (ISH). Sections were collected as follows: 1) facing of the block: the first few sections of the paraffin block were discarded until the first, complete brain section was cut; 2) slides were collected for the analysis of the cortex, each containing 3 7 μm thick tissue sections. Total thickness collected near the midline was approximately 315 μm, centered at an estimated 0.225 mm lateral position; 3) the subsequent 1,200 μm were discarded from the block; 4) additional slides were collected for the analysis of the striatum, each with 3 sections (7 μm thick). Total thickness collected laterally was approximately 315 μm, centered at an estimated 1.725 mm lateral position, 5) The remaining block was stored at room temperature then shipped to the Study Sponsor at the end of the study. Slides were stained for: sense and antisense RNA foci, by chromogenic ISH, poly (GP), poly (GA), and poly-Glycine-Arginine (GR) DPRs, by chromogenic IHC. Slides were counterstained with hematoxylin to label the nuclei. A positive control slide (from an adult mouse injected neonatally with C9-AAV149) and a negative control slide (C57BL6/J, naïve) taken from the medial sagittal plane (cortex) were stained for each of the stain in parallel with the study slides. Slides were scanned on a Hamamatsu Photonics NanoZoomer at 40× magnification. On each slide, one section was selected for uniformity of the staining, and one ROI was selected: one centered on the frontal cortex and one centered on the striatum. Images were subsequently visually inspected in house with the use of HALO Image Analysis Platform (Indica Labs).

Wire hang test. The test was performed at The Jackson Laboratory facility (Bar Harbor, ME) in AAV9-C9orf72 (G4C2)-149 repeats mice at 18 weeks post-AAV5-injection. Mice were placed on a wire grid (grid squares of 1 cm×1 cm, wire diameter of 1 mm). The grid was slightly shaken to increase wire grabbing by the tested mouse, then immediately turned over. Mice were required to hang onto the inverted grid for one minute. Three trials were performed, up to 60 sec each, with a 30 second recovery period between trials. The grid was held 10-12 inches over a box of clean wood shavings.

Contextual learning in fear conditioning test. The test was performed at The Jackson Laboratory facility (Bar Harbor, ME) in AAV9-C9orf72 (G4C2)-149 repeats mice at 18 weeks post-AAV5-injection. Mice in home cage were placed in testing room 1 hour before testing to minimize effects of stress on behavior during testing. Mice were placed in a fear conditioning chamber (Actimetrics, Wilmette, IL), a plexiglass chamber with a stainless-steel floor grid that delivers mild shock to the foot of the mouse. The freezing behavior was captured through the camera mounted in the chamber. The experimental set-up was as follows: on day 1 chambers were set to Context A. The chamber walls were transparent, the chamber was placed in the soundproof chamber which has white walls. Each Mouse underwent 180 seconds baseline period of exposure to the context A. Mice were fear conditioned by using a 30 seconds, 5000 Hz, 90 decibel tone co-terminating with a 0.7 milliampere, 2 seconds foot shock. After the shock the mice were given an additional 180 seconds in the same context before being removed. On day 2 contextual fear memory was evaluated after 24 hours by placing the mouse back in the same context for a 5 minutes exposure session. It was considered freezing behavior when the mouse was completely freezing i.e. completely immobile (respiratory movements are not counted). Freezing scoring was performed for consecutive intervals of 2 seconds each, during day 1 and day 2, for the time spent in the conditioning chamber.

Open field test. The test was performed at The Jackson Laboratory facility (Bar Harbor, ME) in AAV9-C9orf72 (G4C2)-149 repeats mice at 18 weeks post-AAV5-injection. Data were recorded via a sensitive infrared (IR) photobeam three dimensional grid system that was invisible to mice. When the mouse moved or traveled, its body broke the otherwise continuous beam. The automated system then translated the beam breaks into measurements such as distance travelled and number of rearings, as well as where and how long the animal spent time (i.e. center versus perimeter vs corner of arena). The software used was Fusion from Omnitech Electronics Inc. Mice were acclimated to the room for sixty minutes prior to testing. The following readouts were reported: distance travelled, vertical activity counts (i.e. number of rearing), percentage of time spent on the periphery.

Body weight. Body weights of all the AAV9-C9orf72 (G4C2)-149 repeats mice and control mice involved in the study were recorded weekly at The Jackson Laboratory facility (Bar Harbor, ME), with weight to 0.1 resolution.

Statistical analysis. Statistical analyses were performed with GraphPad Prism (version 9.4.1). One-way ANOVA (parametric or non-parametric according to normality of the data) with multiple comparison test was used to identify differences among the groups in the C9orf72 mRNA expression, poly (GP) and poly (GA) amounts, freezing time on the fear conditioning assay, wire hang latency and body weights at 25 weeks in life. Two-way ANOVA followed by Dunnett's multiple comparison test was used to identify statistically significant differences between groups in RNA-sequencing C9orf72 transcripts expression data. Graphs showed average ±S.D. or ±S.E.M. Symbols indicated p values for the post-hoc multiple comparisons, as follows: *: p<0.05, **: p<0.01, ***: p<0.001.

### Results

In vitro

In vitro silencing efficacy of artificial C90 constructs. To evaluate the C90 knockdown efficacy of the C90 and C90AS constructs in vitro, HEK293T cells were co-transfected with Renilla luciferase reporters encoding the C9orf72 target sequences and said C90 constructs. The Firefly luciferase (FL) gene was expressed from the same reporter vector and served as an internal control to correct for transfection efficiency. In the first screening HEK293T cells were co-transfected with 50 or 250 ng of each of the C90 or C90AS constructs, C90-SCRs and pBlueScript (pBS) and 50 ng of Luc-C90-sense or Luc-C90-antisense.

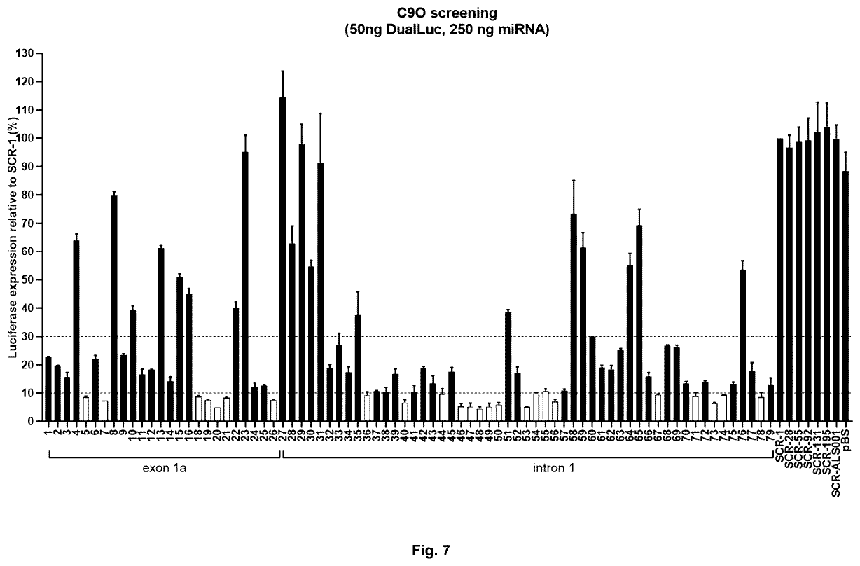

From C901-C9026 constructs designed to target c9orf72 exon 1a, C901, C902, C903, C905-C907, C909, C9011, C9012, C9014, C9018-C9021 and C9024-C9026 induced more than 70% inhibition of the C9orf92 sense luciferase reporter plasmid. From C9027-C9079 constructs designed to target c9orf72 intron 1, C9032, C9033, C9034, C9036-C9050, C9051-C9057, C9060-C9063, C9066-C9075 and C9077-C9079 induced more than 70% inhibition of the C9orf92 sense luciferase reporter plasmid. More than 90% inhibition of the C9orf92 sense luciferase reporter plasmid was achieved by C905, C907, C9018, C9019, C9020, C9021, C9026, C9036, C9040, C9044, C9046-C9050, C9053, C9054, C9056, C9067, C9071, C9073, C9074 and C9078 (FIG. 7, white bars).

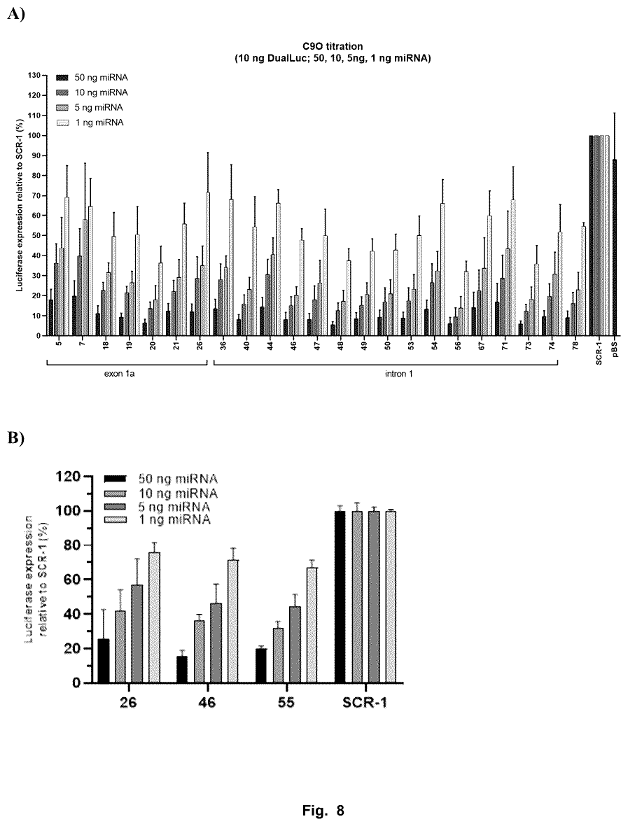

To further determine the potency, the C90 constructs showing more than 90% inhibition of the C9orf72 sense luciferase reporter plasmid were selected for titration experiments (5, 7, 18-21, 26, 36, 40, 44, 46-50, 53, 54-56, 67, 71, 73, 74 and 78). The constructs were co-transfected in HEK293T cells in different concentrations; 1, 5, 10 or 50 ng with 10 ng of c9orf72 sense luciferase reporter plasmid. All the tested C90 constructs showed dose-dependent lowering of the C9orf92 sense luciferase (FIGS. 8.A and B). The lowest miRNA concentration tested of 1 ng (ratio luciferase: miRNA is 10:1) was able to elicit approximately a knockdown between ˜30% and ˜60%. The knockdown measured at increased miRNA concentration was respectively ˜50%-˜ 80% with 5 ng miRNA (ratio luciferase: miRNA is 2:1), ˜60-˜ 80% with 10 ng miRNA (ratio luciferase: miRNA is 1:1) and ˜80-˜ 90% with 50 ng miRNA (ratio luciferase: miRNA is 1:5). The most potent constructs were C9018-C9021 and C9026 targeting c9orf72 exon 1a, and C9040, C9046-53, C9055, C9056 and C9073 targeting c9orf72 intron 1.

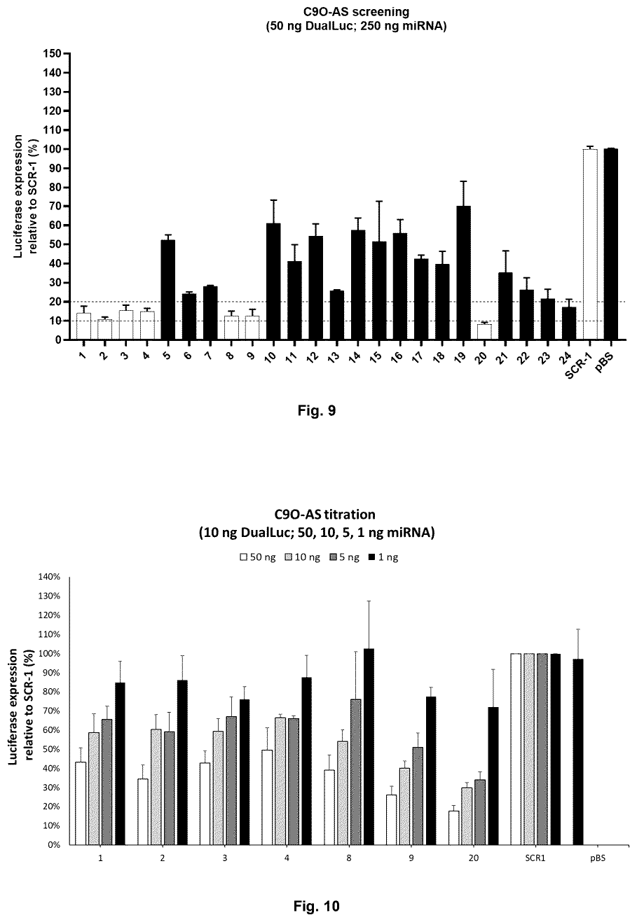

From the constructs designed to target antisense c9orf72, C90-AS1-C90-AS4, C90-AS8, C90-AS9 and C90-AS20 induced more than 80% inhibition of the C9orf72 antisense luciferase reporter plasmid. More than 90% inhibition of the C9orf72 antisense luciferase reporter plasmid was achieved by C90-AS20 (FIG. 9). To further determine the potency, the C90 constructs showing more than 80% inhibition of the C9orf72 antisense luciferase reporter plasmid were selected for titration experiments. The constructs were co-transfected in HEK293T cells in different concentrations; 1, 5, 10 or 50 ng with 10 ng of c9orf72 antisense luciferase reporter plasmid. All the tested C90 constructs showed dose-dependent lowering of the C9orf72 antisense luciferase reporter (FIG. 10). Based on luciferase assays C90-AS20 was the most potent candidate and selected, for further in vitro testing and generation of constructs targeting both the transcripts.

Lowering of endogenous artificial expression in transfected cells. To evaluate the C90 knockdown of intronic C9orf72 mRNA expression in vitro, HEK293T cells were co-transfected with an intronic artificial C9orf72 construct harboring 99 repeats and said C90 constructs. The knockdown of the intronic C9orf72 expression in HEK293T cells was measured by RT-QPCR on transfected cells.

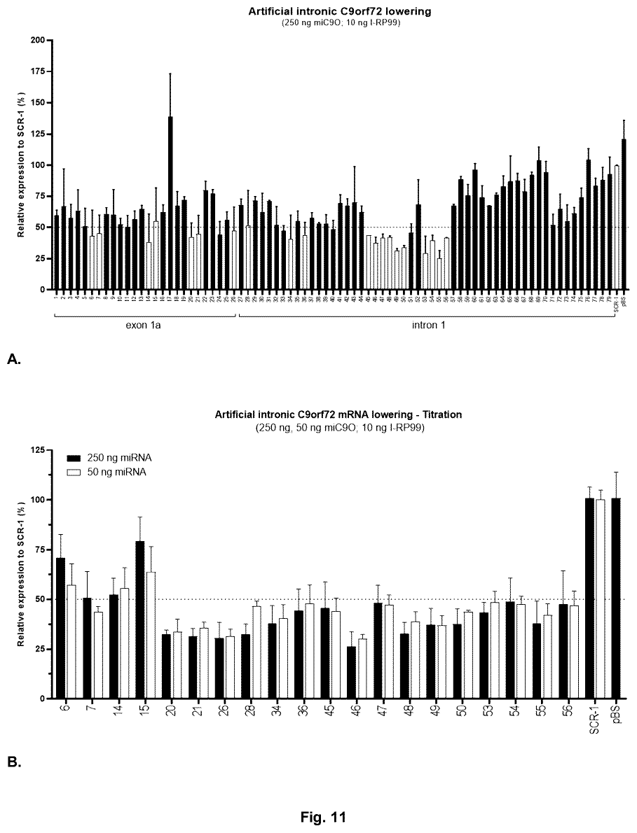

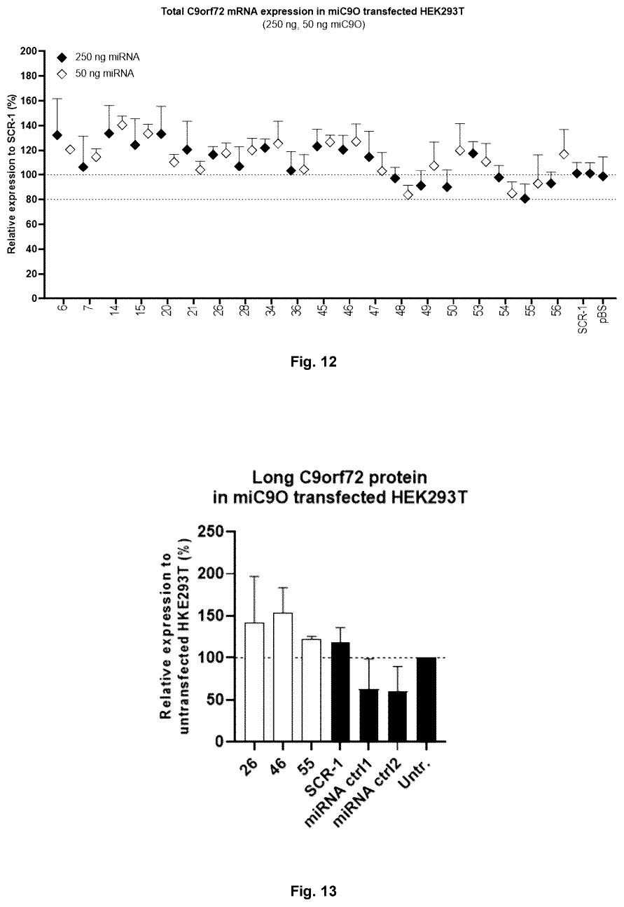

From C901-C9026 constructs designed to target C9orf72 exon 1a, transfection of 250 ng of miRNA plasmid resulted in a decrease of intronic c9orf72 mRNA expression of more than 50% by C906, C907, C9014, C9015, C9020, C9021 and C9025 (FIG. 11.A). From C9027-C9079 constructs designed to target C9orf72 intron 1, transfection of 250 ng of miRNA plasmid resulted in a decrease of intronic c9orf72 mRNA expression of more than 50% by C9028, C9034, C9036, C9045-C9050 and C9053-C9056 (FIG. 11.A). To further determine the potency, the C90 constructs showing more than 50% lowering of the intronic C9orf72 mRNA were selected for titration experiments. The constructs were co-transfected in HEK293T cells in two different concentrations; 50 or 250 ng with 10 ng of artificial intronic C9orf72 construct harboring 99 repeats. The results showed that most of the selected C90 construct (except C906, C9014 and C9015) lowered more than 50% the expression of intronic C9orf72 at both tested DNA concentrations (FIG. 11.B). The most promising constructs showing highest knockdown are C9020, C9021, and C9026 (targeting C9orf72 exon 1a) and C9046, C9048 and C9055 (targeting c9orf72 intron 1). The total C9orf72 endogenous expression in HEK293T is minimally affected (maximum 20% lowering), confirming that C9Os are not targeting the healthy isoform of C9orf72 in HEK293T cells (FIG. 12).

Selection of the C90 sense candidates for further in vitro and in vivo testing was performed based on the C9orf72 sense luciferase lowering data and the artificial intronic C9orf72 mRNA lowering, and C9026, C9046 and C9055 were chosen. Selection of the C90 antisense candidate for the generation of constructs targeting both the transcripts was performed based solely on the C9orf72 sense luciferase lowering, and C90-AS20 was chosen.

Protein analysis HEK293T. The C90 candidates 26, 46 and 55 have been shown to be able to efficiently lower intronic C9orf72 expression in transfected HEK392T cells, without decreasing the expression of the total pool of C9orf72 transcripts. To provide confirmation also at protein level, the long isoform C9orf72 protein expression was analyzed in cell lysates of HEK293T transfected with 250 ng of C9026, 46 and 55, C90-SCR1 and untransfected cells. As lowering positive controls, two miRNA constructs with the ability to target all the C9orf72 transcripts were included. The results showed that C9026, 46 and 55 and C90-SCR1 are not lowering the endogenous expression of C9orf72 protein in HEK293T compared to untransfected cells (FIG. 13). The two miRNA constructs that are targeting all C9orf72 transcripts were used as positive control and resulted in ˜40% of the long isoform C9orf72 protein lowering upon transfection.

In vitro silencing efficacy of V1S/V2S constructs. To evaluate the knockdown efficacy of V1S (V1S-13-V1S-23) and V2S (V2S-42-V2S-53) constructs in vitro, HEK293T cells were co-transfected with Renilla luciferase reporters encoding the sense or antisense C9orf72 target sequences and said linC90 constructs. The Firefly luciferase (FL) gene was expressed from the same reporter vector and served as an internal control to correct for transfection efficiency. In the first screening HEK293T cells were co-transfected with 50 fmol of each of the V1S/V2S constructs, V1S/V2S-SCRs and pBlueScript (pBS) and 8.2 fmol of Luc-C90-sense and Luc-C90-antisense.

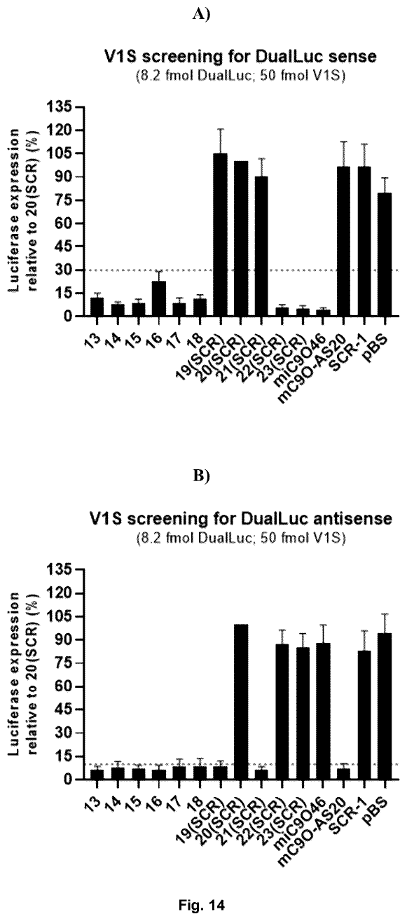

Transfection of 50 fmol V1S constructs of resulted in a decrease of more than 70% of the C9orf72 sense luciferase reporter expression. The V1S constructs showing the most potent sense luciferase reporter lowering are V1S-13-V1S-15 (FIG. 14.A). Transfection of 50 fmol V1S constructs resulted in a decrease of more than 90% of the C9orf72 antisense luciferase reporter expression (FIG. 14.B). Therefore, to further determine the potency, the V1S-13-V1S-15 were selected for titration experiments.

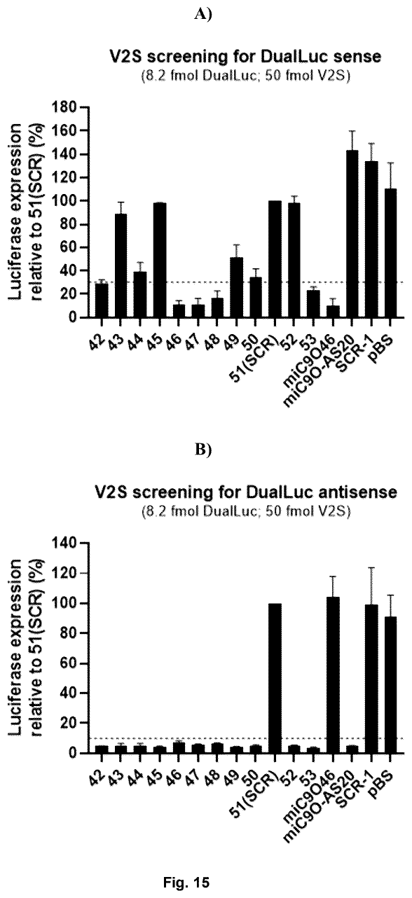

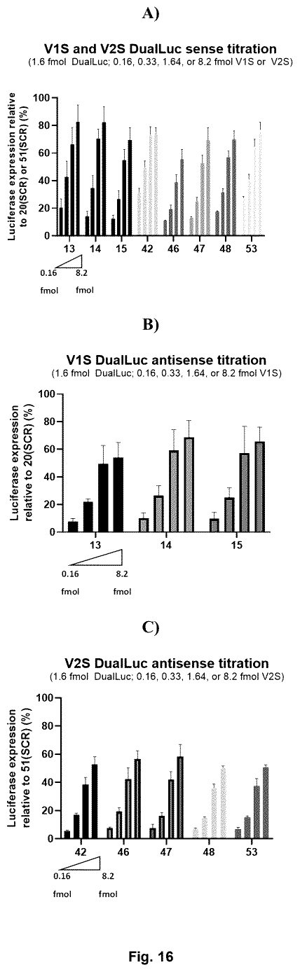

Transfection of 50 fmol of the V2S constructs resulted in more than 70% decrease of the C9orf92 sense luciferase reporter expression for in V2S-42, V2S-46-V2S-48 and V2S-53 (FIG. 15.A). All other V2S constructs resulted in a knockdown of more than 90% of the C9orf92 antisense luciferase reporter upon transfection (FIG. 15.B). Therefore, to further determine the potency, the V2S-42, V2S-46-V2S-48 and V2S-53 were selected for titration experiments.

To further determine the potency, the selected V1S/V2S constructs (13-15, 42, 46-48 and 53) were co-transfected in HEK293T cells in different concentrations; 0.16, 0.33, 1.64 or 8.2 fmol with 1.6 fmol of C9orf72 sense or antisense luciferase reporter plasmid. All the tested C90 constructs showed dose-dependent lowering of the C9orf72 sense and luciferase reporters. The sense luciferase lowering results highlighted that V2S-46 [miR-144 (mQS55) 5p-miR-451 (mQAS20)] was the most potent V1S/V2S construct (˜50%-˜ 80% according to tested amount of miRNA; FIG. 16.A). The antisense luciferase lowering results showed comparable antisense luciferase lowering among the tested V1S/V2S (˜40%-˜ 90% according to tested amount of miRNA; FIGS. 16.B and C). For further in vitro and in vivo testing the V2S number 46 and 48 (harboring a different sense guide and showing potent sense and antisense luciferase lowering), were chosen.

Expression levels of C90 in transfected HEK293T cells. The expression level of the mature miRNAs in HEK293T transfected with 50 ng of C9026, C9046 and C9055 was quantified based on the number of the total reads annotated by using miRBase and the pri-miRNA sequence of interest.

C9026 ranked as the 56th (experiment 1) and 26th (experiment 2) most abundant mature miRNA when transfected in HEK293T (Table 1). All the processed forms of C9026 counted for 0.06% and 0.42% (respectively, experiment 1 and 2) of the total annotated reads. C9046 was one of the most abundant miRNA in transfected HEK293T, being the fifth and sixth most expressed when 50 ng of DNA was transfected (Table 1) and all the processed forms of C9046 counted for 2.2% and 3.09% (respectively, experiment 1 and 2) of the total annotated reads. C9055 ranked as the 35th (experiment 1) and 44th (experiment 2) most abundant mature miRNA found in HEK293T (Table 1). All the processed forms of C9046 counted for 0.22% and 0.14% (respectively, experiment 1 and 2) of the total annotated reads. In both set of experiments the results showed that the expression levels of the transfected miRNAs are not exceeding those of the endogenous HEK293T miRNAs.

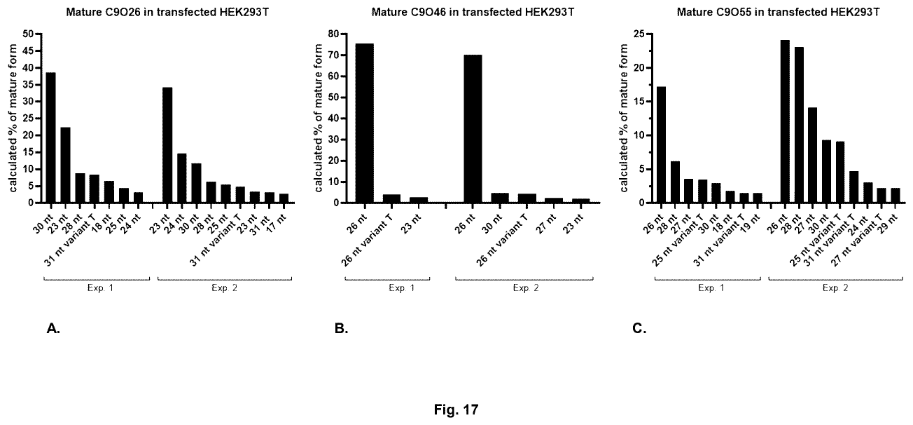

Processing of C90 constructs upon transfection in HEK293T cells (NGS data). The miRNAs processing was also investigated by alignment of the reads to the pri-miRNA sequences. The top 20 most abundant mature forms obtained from the annotation process were considered for graphical purposes and set to 100% (reads represented with less than 2% are not shown). The length of the most abundant form for C9026 were 30 nts and 23 nts (FIG. 17.A), the most length of the most abundant form for C9046 and C9055 was 26 nts (FIGS. 17B and C). Observed mismatches with the reference sequence consisted in sequence modification at the 3′ in which thymine (uracil) was added to the mature guide sequences. This is in accordance with previously published data on 3′ end editing events in various cell lines and tissues (Landgraf et al., 2007 Cell129 (7): 1401-14). However, the exact roles of mono-uridylation and mono-adenylation still needs to be determined.

TABLE 4
Expression level of the top 50 or 56 mature miRNAs in HEK293T transfected with 50 ng of C9O26, C9O46 and C9O55

| | Mature C9O26% | | | | Mature C9O46% | | | | Mature C9O55% | | | |
|---|---|---|---|---|---|---|---|---|---|---|---|---|
| Ranking | Exp #1 | % | Exp #2 | % | Exp #1 | % | Exp #2 | % | Exp #1 | % | Exp #2 | % |
| 1 | mir-10a | 14.16 | mir-10a | 10.64 | mir-10a | 11.01 | mir-10a | 10.11 | mir-10a | 10.84 | mir-10a | 12.42 |
| 2 | mir-10b | 7.20 | mir-20a | 4.87 | mir-10b | 4.20 | mir-10b | 3.79 | mir-10b | 4.21 | mir-10b | 5.37 |
| 3 | mir-30d | 2.52 | mir-30d | 4.11 | mir-20a | 3.83 | mir-30d | 3.57 | mir-30d | 3.62 | mir-30d | 3.28 |
| 4 | mir-7-1 | 1.65 | mir-10b | 3.99 | mir-30d | 3.54 | C9O46 | 3.09 | mir-20a | 3.37 | mir-20a | 2.10 |
| 5 | mir-7-2 | 1.59 | mir-17 | 2.41 | C9O46 | 2.20 | mir-20a | 3.00 | mir-17 | 1.88 | mir-7-1 | 1.82 |
| 6 | mir-7-3 | 1.59 | mir-30a | 1.74 | mir-17 | 1.99 | mir-17 | 1.64 | mir-21 | 1.29 | mir-7-2 | 1.74 |
| 7 | mir-21 | 1.06 | mir-21 | 1.52 | mir-21 | 1.34 | mir-21 | 1.24 | mir-30a | 1.08 | mir-7-3 | 1.74 |
| 8 | mir-20a | 0.72 | mir-7-1 | 0.98 | mir-7-1 | 1.19 | mir-30a | 1.12 | mir-7-1 | 1.05 | mir-21 | 1.41 |
| 9 | mir-99b | 0.61 | mir-7-2 | 0.94 | mir-7-2 | 1.13 | mir-7-1 | 1.02 | mir-7-2 | 1.00 | mir-17 | 1.15 |
| 10 | mir-532 | 0.57 | mir-7-3 | 0.93 | mir-7-3 | 1.13 | mir-7-2 | 0.98 | mir-7-3 | 1.00 | mir-30a | 0.78 |
| 11 | mir-30a | 0.56 | mir-191 | 0.79 | mir-30a | 1.12 | mir-7-3 | 0.98 | mir-1246 | 0.82 | mir-26a-1 | 0.77 |
| 12 | mir-183 | 0.53 | mir-182 | 0.78 | mir-191 | 0.80 | mir-191 | 0.69 | mir-191 | 0.81 | mir-26a-2 | 0.77 |
| 13 | mir-182 | 0.53 | mir-93 | 0.76 | mir-182 | 0.78 | mir-26a-1 | 0.68 | mir-26a-1 | 0.79 | mir-182 | 0.71 |
| 14 | mir-26a-2 | 0.50 | mir-26a-1 | 0.66 | mir-26a-1 | 0.73 | mir-26a-2 | 0.68 | mir-26a-2 | 0.79 | let-7g | 0.69 |
| 15 | mir-26a-1 | 0.50 | mir-26a-2 | 0.66 | mir-26a-2 | 0.73 | mir-182 | 0.66 | mir-182 | 0.79 | mir-183 | 0.58 |
| 16 | mir-17 | 0.43 | mir-1246 | 0.56 | mir-93 | 0.73 | mir-93 | 0.58 | mir-93 | 0.72 | mir-532 | 0.55 |
| 17 | let-7g | 0.38 | mir-183 | 0.53 | let-7g | 0.66 | mir-1260a | 0.54 | let-7g | 0.60 | mir-191 | 0.52 |
| 18 | let-7f-2 | 0.37 | mir-1260a | 0.49 | mir-183 | 0.58 | mir-183 | 0.50 | mir-183 | 0.58 | mir-99b | 0.51 |
| 19 | let-7f-1 | 0.34 | mir-340 | 0.47 | mir-99b | 0.49 | let-7g | 0.48 | mir-99b | 0.53 | let-7f-2 | 0.47 |
| 20 | mir-1260a | 0.29 | let-7g | 0.47 | mir-532 | 0.44 | mir-99b | 0.43 | mir-532 | 0.47 | mir-93 | 0.45 |
| 21 | mir-192 | 0.26 | mir-532 | 0.45 | mir-1260a | 0.42 | mir-532 | 0.42 | mir-1260a | 0.46 | let-7f-1 | 0.43 |
| 22 | mir-196b | 0.26 | mir-99b | 0.42 | mir-340 | 0.41 | mir-340 | 0.37 | mir-340 | 0.41 | mir-196b | 0.42 |
| 23 | mir-191 | 0.26 | C9O26 | 0.42 | let-7f-2 | 0.40 | mir-196b | 0.31 | mir-196b | 0.37 | mir-340 | 0.40 |
| 24 | mir-99a | 0.25 | mir-30e | 0.41 | let-7f-1 | 0.38 | mir-30e | 0.30 | let-7f-2 | 0.32 | mir-1260a | 0.33 |
| 25 | let-7i | 0.24 | mir-186 | 0.40 | mir-196b | 0.36 | mir-192 | 0.30 | mir-30e | 0.32 | let-7i | 0.28 |
| 26 | mir-423 | 0.22 | mir-374b | 0.35 | mir-30e | 0.31 | let-7f-2 | 0.28 | mir-186 | 0.31 | mir-30e | 0.25 |
| 27 | mir-340 | 0.20 | mir-484 | 0.30 | mir-186 | 0.31 | mir-186 | 0.27 | let-7f-1 | 0.30 | mir-192 | 0.25 |
| 28 | mir-93 | 0.17 | mir-378i | 0.30 | mir-374b | 0.30 | let-7f-1 | 0.26 | mir-484 | 0.29 | mir-99a | 0.25 |
| 29 | mir-130b | 0.14 | mir-196b | 0.27 | mir-423 | 0.28 | mir-378i | 0.26 | mir-374b | 0.28 | mir-423 | 0.23 |
| 30 | mir-30e | 0.13 | mir-192 | 0.27 | mir-484 | 0.28 | mir-484 | 0.26 | mir-423 | 0.27 | mir-378i | 0.22 |
| 31 | mir-629 | 0.12 | let-7f-2 | 0.26 | mir-1246 | 0.27 | mir-1246 | 0.26 | mir-378i | 0.27 | mir-26b | 0.20 |
| 32 | mir-484 | 0.12 | mir-425 | 0.25 | mir-192 | 0.25 | mir-425 | 0.24 | mir-192 | 0.25 | mir-186 | 0.20 |
| 33 | mir-425 | 0.12 | let-7f-1 | 0.25 | mir-378i | 0.22 | mir-423 | 0.23 | mir-30c-1 | 0.22 | mir-30c-2 | 0.19 |
| 34 | let-7a-3 | 0.11 | mir-423 | 0.24 | mir-30c-1 | 0.21 | mir-99a | 0.23 | mir-30c-2 | 0.22 | mir-629 | 0.19 |
| 35 | let-7a-1 | 0.11 | mir-185 | 0.21 | mir-30c-2 | 0.21 | mir-374b | 0.19 | C9O55 | 0.22 | mir-30c-1 | 0.19 |
| 36 | let-7a-2 | 0.11 | mir-30c-2 | 0.19 | let-7i | 0.20 | mir-30c-2 | 0.19 | mir-629 | 0.21 | mir-484 | 0.18 |
| 37 | mir-30c-2 | 0.11 | mir-30c-1 | 0.19 | mir-629 | 0.20 | mir-30c-1 | 0.19 | let-7i | 0.20 | mir-130b | 0.16 |
| 38 | mir-30c-1 | 0.10 | mir-629 | 0.18 | mir-425 | 0.19 | let-7i | 0.18 | mir-185 | 0.20 | mir-374b | 0.15 |
| 39 | mir-146b | 0.10 | mir-106b | 0.17 | mir-185 | 0.19 | mir-26b | 0.17 | mir-99a | 0.19 | let-7a-3 | 0.15 |
| 40 | mir-186 | 0.10 | mir-769 | 0.17 | mir-26b | 0.18 | mir-629 | 0.16 | mir-125a | 0.18 | let-7a-1 | 0.15 |
| 41 | mir-9-2 | 0.10 | mir-26b | 0.17 | mir-125a | 0.18 | mir-185 | 0.16 | mir-26b | 0.18 | mir-125a | 0.15 |
| 42 | mir-378i | 0.10 | let-7i | 0.16 | mir-99a | 0.15 | mir-125a | 0.16 | mir-425 | 0.18 | let-7a-2 | 0.15 |
| 43 | mir-9-3 | 0.10 | mir-18a | 0.15 | let-7a-3 | 0.14 | mir-1260b | 0.14 | mir-130b | 0.15 | mir-185 | 0.14 |
| 44 | mir-9-1 | 0.10 | mir-30b | 0.14 | let-7a-1 | 0.14 | mir-130b | 0.12 | mir-1260b | 0.13 | C9O55 | 0.14 |
| 45 | mir-26b | 0.10 | mir-99a | 0.14 | mir-130b | 0.14 | mir-769 | 0.12 | mir-18a | 0.12 | mir-425 | 0.13 |
| 46 | mir-148a | 0.09 | mir-125a | 0.14 | let-7a-2 | 0.14 | mir-18a | 0.11 | mir-769 | 0.12 | mir-9-2 | 0.13 |
| 47 | mir-769 | 0.09 | mir-339 | 0.13 | mir-769 | 0.13 | mir-106b | 0.11 | let-7a-3 | 0.12 | mir-9-1 | 0.13 |
| 48 | mir-125a | 0.07 | mir-374a | 0.13 | mir-18a | 0.13 | mir-32 | 0.10 | let-7a-1 | 0.12 | mir-9-3 | 0.13 |
| 49 | mir-450b | 0.07 | mir-1260b | 0.13 | mir-1260b | 0.11 | mir-148a | 0.10 | let-7a-2 | 0.11 | mir-196a-2 | 0.11 |
| 50 | mir-1260b | 0.07 | mir-130b | 0.10 | mir-106b | 0.11 | mir-339 | 0.10 | mir-339 | 0.11 | mir-769 | 0.11 |
| 51 | mir-455 | 0.07 | | | | | | | | | | |
| 52 | mir-589 | 0.06 | | | | | | | | | | |
| 53 | mir-185 | 0.06 | | | | | | | | | | |
| 54 | mir-1269b | 0.06 | | | | | | | | | | |
| 55 | mir-374b | 0.06 | | | | | | | | | | |
| 56 | C9O26 | 0.06 | | | | | | | | | | |

AAV5-C90_and_AAV5-V1S/V2S transduction in HEK293T cells. To investigate the ability of obtained AAV5-C9026, AAV5-C9046, AAV5-C9055, AAV5-C90-SCR1, AAV5-V2S-46, AAV5-V2S-48 and AAV5-V2S-SCR to transduce and deliver the packaged expression cassette, HEK293T cells were transduced (n=2) at a Multiplicity of Infection (MOI) of 1E+06, 1E+05 and 1E+04 gc/cell. Vector DNA was quantified by QPCR. The results showed a dose-dependent increase in detected vector genome DNA copies (Table 5).

TABLE 5
Vector genome DNA from transduced HEK293T cells
with AAV5-C9Os and AAV5-V2S constructs.

| AAV5 vector | MOI 1E+04 | MOI 1E+05 | MOI 1E+06 |
|---|---|---|---|
| encoding: | gc/μg DNA | gc/μg DNA | gc/μg DNA |
| C9O26 | 6.1 + 06 | 4.9E+07 | 1.7E+08 |
| C9O46 | 1E+07 | 5.9E+07 | 2.3E+08 |
| C9O55 | 1.6E+07 | 8E+07 | 2.6E+08 |
| C9O-SCR1 | 1.3E+07 | 8E+07 | 1.5E+08 |
| V2S-46 | 9.8 + 06* | 7.2E+07* | 3.2E+08 |
| V2S-48 | 7.3 + 06* | 6.5E+07* | 3.2E+08* |
| V2S-SCR | 1.2 + 07 | 8.8E+07 | 2.5E+08 |

*data from one transduction experiment.

Lowering of mutant C9orf72 in motor neurons and differential expressed genes (DEGs) by C9046. To test the C9046 silencing of mutant C9orf72 mRNAs expression in motor neurons, hiPSC cells from an ALS patient harboring approximately 800 repeats in the C9ORF72 gene were used to generate spinal motor neurons [identified here as ALS hiPSC-derived motor neurons or ALS motor neurons (ALS MNs)]. Such cells were transduced with AAV6-C9046, AAV6-C90-SCR1. The untransduced control (UTC) was treated with Dulbecco's phosphate-buffered saline. The expression of C9orf72 mRNA isoforms was measured by RT-QPCR and RNA-sequencing (RNA-seq). Additionally, differential expressed genes (DEGs) between C9046 and the controls were assessed by RNA-seq and bioinformatic analysis in ALS and control transduced motor neurons.

Before the aforementioned experiment, the transduction efficiency of several AAV serotypes expressing enhanced green fluorescent protein (eGFP) was assessed by transducing hiPSC-derived motor neurons, by imaging for eGFP expression and by calculating the percentage of motor neurons positive for eGFP expression though fluorescence-activated cell sorting analysis. The AAV serotype 6 (AAV6) showed the highest percentage of motor neurons positive for eGFP expression (˜50% at MOI 1.00E+07 GC/cell) and therefore AAV6 was used to perform transduction experiment in hiPSC-derived motor neurons.

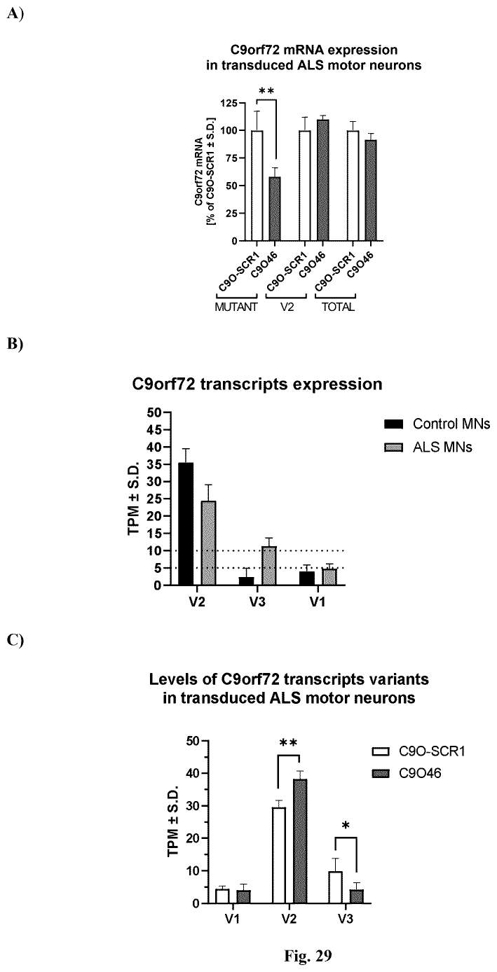

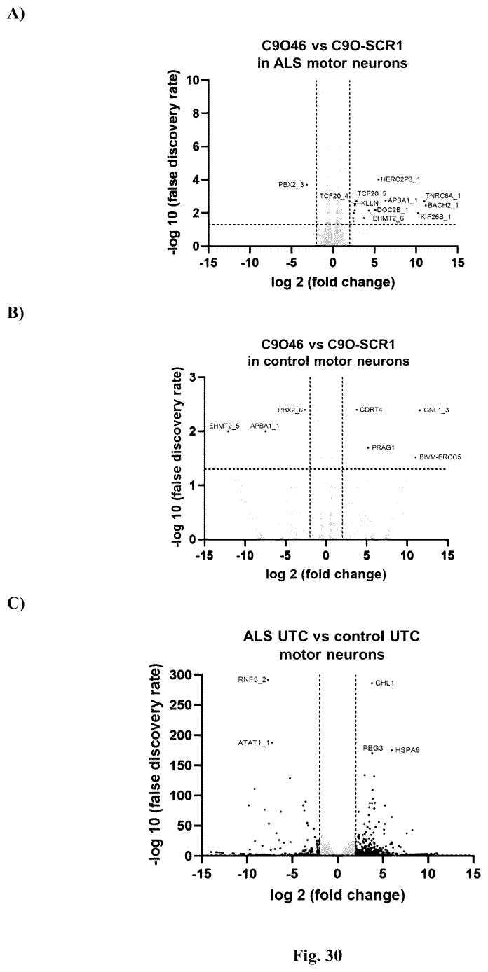

Transduction of ALS hiPSC-derived motor neurons with AAV6-C9046 resulted in a significant lowering (p<0.01) of the mutant C9orf72 mRNA expression compared to C90-SCR1, while the V2 (healthy) and total C9orf72 mRNA expression was unchanged (FIG. 29.A). The expression of C9orf72 mRNA variants was assessed by RNA-sequencing analysis in healthy and ALS hiPSC-derived motor neurons. In the ALS pathology C9orf72 V1 and V3 transcripts contain the mutation (repeat expansion). The results of this experiment showed that the ALS motor neurons considered for this experiment had increased C9orf72 V3 transcript expression compared to healthy motor neurons, while C9orf72 V1 was unchanged (FIG. 29.B). In samples from ALS motor neurons the RNA-sequencing data showed a lower number of transcripts per million (TPM) for V3 C9orf72 in C9046 treated cells compared to C90-SCR1 (p<0.05). The lowest expressed V1 C9orf72 showed no change in the number of TPM, while a higher number of TPM for the healthy V2 C9orf72 was detected in samples from C9046 treated cells (p<0.01) compared to C90-SCR1 (FIG. 29.C). In silico analysis of possible off-target gene transcripts was performed for C9046 miRNA sequence, considering for the Blast with analysis guide nucleotides g2-g15 and g2-g17. Only one transcript was found having a complete match with guide nucleotides g2-g15 and g2-g17 and corresponding to a long non-coding RNA (antisense to MACROD2, Ensembl ID: ENST00000656415). Perfect pairing of guide nucleotides g2-g17 represents a high risk of off-target transcript, while perfect pairing of guide nucleotides g2-g15 represents a lower risk of off-target transcript. Additionally, the number of transcripts having no mismatch or one mismatch within or outside the miRNA seed region (guide nucleotides 2-8) were identified. Analyzing the nucleotides g2-g17 of the C9046 miRNA guide, two transcripts belonging to the same gene were found to have complete seed sequence recognition and a mismatch outside the seed sequence (phospholipase A2 receptor 1, PLA2R1, Ensembl IDs: ENST00000283243, ENST00000392771). Analyzing the nucleotides g2-g15 of C9046 miRNA guide a higher number of transcripts were found to have complete seed sequence recognition and a mismatch outside the seed sequence (see Table 8 for Ensembl IDs). The RNA-seq analysis showed that a higher number of modulated genes in ALS motor neurons is related to the AAV6 transduction event (85 genes C90-SCR1 vs UTC) compared to the expression of C9046 miRNA (17 genes C9046 vs C90-SCR1). Similarly, in control motor neurons the highest number of DEGs was found when the results of C90-SCR1 and C9046 groups were compared to UTC (respectively 31 and 92 DEGs), eight DEGs were identified when comparing C9046 with C90-SCR1. Gene ID list of the statistical significant DEGs in the contrast C9046 vs C90-SCR1 respectively in ALS and control motor neurons are found in Table 6 and Table 7. Volcano plots were generated for the comparisons C9046 vs C90-SCR1 groups (FIG. 30.A and FIG. 30.B) and UTC group (FIG. 30.C) in ALS and control motor neurons. The volcano plots are visualizing the effect size [log 2 (fold change)] against significance [-log 10 (p-values)] for top ten up and down regulated gene transcripts. The non-significant genes are displayed in light gray, vertical and horizontal dashed lines represent the fold change and p-value thresholds applied, respectively. The gene ontology (GO) analysis was used to perform enrichment analysis on two sets of significantly DEGs found when comparing C9046 and C90-SCR1 in ALS and control motor neurons. GO comprises three orthogonal ontologies, biological processes, molecular function and cellular component. No significant GO terms were found enriched for biological processes molecular function or cellular component in the analyzed gene sets. The in silico predicted transcripts were found not significantly differentially expressed in the RNA-seq data from AAV6-C9046 vs AAV6-C90-SCR1 transduced ALS and control motor neurons.

TABLE 6
DEGs in ALS motor neurons treated C9O46 vs C9O-
SCR1 listed by ascending fold change value.

| | Max group | Log₂ fold | Fold | | FDR |
|---|---|---|---|---|---|
| Gene ID | mean | change | change | P-value | p-value |
| PBX2_3 | 3.67 | −3.16 | −8.95 | 1.40e−7 | 1.96e−4 |
| TCF20_3 | 0.81 | 2.43 | 5.37 | 1.08e−4 | 0.02 |
| TCF20_2 | 0.8 | 2.45 | 5.48 | 1.57e−4 | 0.03 |
| TCF20_6 | 0.87 | 2.53 | 5.79 | 5.39e−5 | 0.01 |
| TCF20_7 | 0.84 | 2.61 | 6.11 | 2.33e−5 | 7.07e−3 |
| TCF20_1 | 0.83 | 2.63 | 6.18 | 9.70e−6 | 3.47e−3 |
| TCF20_4 | 0.82 | 2.63 | 6.19 | 6.54e−6 | 2.80e−3 |
| KLLN | 0.94 | 2.67 | 6.37 | 7.49e−6 | 2.83e−3 |
| TCF20_5 | 0.85 | 2.81 | 7 | 4.04e−6 | 1.94e−3 |
| CCDC92_1 | 2.78 | 3.73 | 13.25 | 1.29e−4 | 0.02 |
| EHMT2_6 | 0.82 | 4.29 | 19.55 | 2.55e−5 | 7.26e−3 |
| DOC2B_1 | 0.79 | 5.07 | 33.58 | 2.14e−5 | 6.64e−3 |
| HERC2P3_1 | 0.64 | 5.45 | 43.77 | 5.49e−8 | 9.58e−5 |
| APBA1_1 | 0.84 | 6.31 | 79.6 | 3.00e−6 | 1.75e−3 |
| KIF26B_1 | 0.34 | 10.23 | 1204.30 | 4.33e−5 | 0.01 |
| TNRC6A_1 | 1.01 | 10.98 | 2023.14 | 3.96e−6 | 1.94e−3 |
| BACH2_1 | 1.42 | 11.14 | 2262.99 | 1.00e−5 | 3.50e−3 |

TABLE 7
DEGs in control motor neurons treated C9O46 vs
C9O-SCR1 listed by ascending fold change value.

| | Max group | Log₂ fold | Fold | | FDR |
|---|---|---|---|---|---|
| Gene ID | mean | change | change | P-value | p-value |
| EHMT2_5 | 4.11 | −12.1 | −4379.94 | 6.46e−6 | 0.01 |
| APBA1_1 | 1.47 | −7.5 | −180.84 | 4.69e−6 | 0.01 |
| PBX2_6 | 6.19 | −2.63 | −6.17 | 6.06e−7 | 3.98e−3 |
| CDRT4 | 3.84 | 3.78 | 13.74 | 3.94e−7 | 3.98e−3 |
| PRAG1 | 0.46 | 5.16 | 35.71 | 1.20e−5 | 0.02 |
| BIVM-ERCC5 | 1.9 | 11.03 | 2090.42 | 1.82e−5 | 0.03 |
| GNL1_6 | 1.98 | 11.49 | 2884.18 | 1.22e−6 | 4.04e−3 |
| GNL1_3 | 2.07 | 11.56 | 3012.57 | 1.05e−6 | 4.04e−3 |

TABLE 8
In silico predicted off-target transcripts (symbol
and gene ID) with mismatch outside 2-15 nucleotides
sequence of C9O46 miRNA guide.

| # | Symbol | Ensembl Gene ID |
|---|---|---|
| 1 | MECP2 | ENSG00000169057 |
| 2 | USP8 | ENSG00000138592 |
| 3 | novel transcript | ENSG00000286770 |
| 4 | TM2D2 | ENSG00000169490 |
| 5 | ATXN8OS | ENSG00000230223 |
| 6 | CYYR1 | ENSG00000166265 |
| 7 | EFHC1 | ENSG00000096093 |
| 8 | ARMCX5-GPRASP2 | ENSG00000286237 |
| 9 | GPRASP3 | ENSG00000198908 |
| 10 | HECW2 | ENSG00000138411 |
| 11 | SNX19 | ENSG00000120451 |
| 12 | novel transcript | ENSG00000280383 |
| 13 | SERPING1 | ENSG00000149131 |
| 14 | novel transcript | ENSG00000283235 |
| 15 | novel transcript | ENSG00000248596 |
| 16 | novel transcript | ENSG00000286879 |
| 17 | ARMC9 | ENSG00000135931 |
| 18 | C12orf73 | ENSG00000204954 |
| 19 | DTWD1 | ENSG00000104047 |
| 20 | KAT6A | ENSG00000083168 |
| 21 | PLA2R1 | ENSG00000153246 |
| 22 | LIMS2 | ENSG00000072163 |
| 23 | SIRPD | ENSG00000125900 |
| 24 | AP2S1 | ENSG00000042753 |
| 25 | IL17RA | ENSG00000177663 |
| 26 | novel transcript | ENSG00000257494 |
| 27 | HMGN1P9 | ENSG00000248377 |
| 28 | FGFR2 | ENSG00000066468 |

In vitro off-targets. Additional experiments are performed using ALS and control hiPSC-derived motor neurons to assess differential expressed genes after C9046 transduction, the appropriate negative controls (i.e. scrambled miRNA) and several doses. Further experiments include additional cell types such as astrocytes, microglia and hepatocytes.

### In Vivo

### Mouse Studies

### Processing and Abundance of Mature C9026, C9046 and C9055 in Mouse Brain.

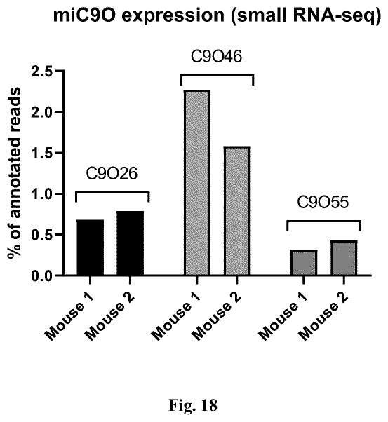

To study miRNA processing of C9026, C9046 and C9055 in mice upon intrastriatal injection of AAV5 vectors, 6-8 weeks old male wild type C57/BL6JRj mice (n=2) received a bilateral infusion of 1.5E+11 gc of AAV5-C9026, AAV5-C9046 and AAV5-C9055 vectors. At week 4 post-infusion animals were sacrificed, brain was collected from the mice, snap frozen and was processed for total RNA isolation and small RNA sequencing (not shown). Small RNA sequencing analysis was performed by GenomeScan B.V. (Nextflex small RNA kit) on total RNA samples prepared from the striatum of AAV5-C9026, AAV5-C9046 and AAV5-C9055 injected mice. The miRNA expression from C9026, C9046 and C9055 was obtained by calculating the percentage of reads mapping to the miRNA sequence in relationship to the total annotated reads mapping to the mouse miRNA database (miRbase). As shown in FIG. 18, the most expressed miRNA is C9046 (1.5%-2.5%), followed by C9026 (0.5%-1%) and C9055 (<0.5%). The expression level of mature C9026, C9046 and C9055 miRNA guides relative to the endogenous expressed miRNAs was determined. The most expressed C9046 in the two analysed sample ranked as the 10th and 13th most abundant miRNA, respectively, followed by C9026 as 26th and 31st most abundant miRNA and C9055 as 39th and 45th. The expression and abundance data revealed that the expression of mature C90 is within the levels of endogenous miRNAs, and hence there is a low risk of interference with endogenous miRNA pathways.

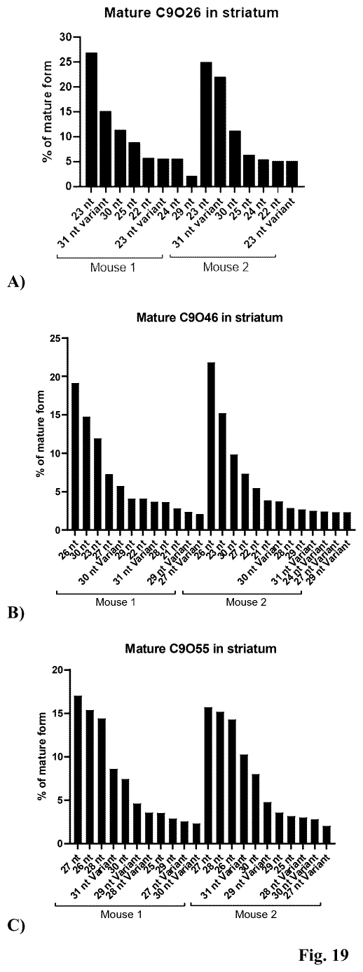

Small RNA sequencing allows to investigate the processing profile of expressed mature miRNAs. In FIG. 19 are shown the length of mature miRNA isoforms identified and their abundance calculated as the percentage of total reads mapping to the reference C90 sequence. The top three most abundant mature isoforms for C9026 were 23, 31 and 30 nucleotides long, followed by 25, 22, 23, 24 and 29 nucleotides long forms with minor shift in the abundancy within the two analysed samples (FIG. 19.A). AAV5-C9046 and AAV5-C9055 showed a wider range of expressed isoforms. In FIG. 19.B the top three most abundant mature isoforms for C9046 were 26, 30 or 23 nucleotides long, followed by 27, 29, 22, 31, 28, 21 nucleotides long forms with minor shift in the abundancy within the two analysed samples. FIG. 19.C shows the C9055 mature guide processing with the top three most abundant mature isoforms being 27, 26 or 28 nucleotides long, followed by 31, 30, 29, 28, 25 and 27 nucleotides long forms with minor shift in the abundancy within the two analysed samples. Observed mismatches with the reference sequence (indicated in FIG. 19 as “variant”) consisted in sequence modification at the 3′ in which thymine (uracil) was added to the mature guide sequences. This is in accordance with previously published data on 3′ end editing events in various cell lines and tissues (Landgraf et al., 2007 Cell 129 (7): 1401-14). However, the exact roles of mono-uridylation and mono-adenylation still need to be determined.

The mature guide strand sequence of the miR-451 is generated by cleaving the 3′arm between nucleotide 10-11 from the stem origin which yields a 30 nucleotide long intermediate that is subsequently trimmed by poly (a)-specific ribonuclease (PARN) into a mature ˜23 nucleotide long active molecule (Cheloufi et al. 2010 465 (7298): 584-9 and Yang et al., 2010 Proc Natl Acad Sci U.S.A. 107 (34): 15163-8). In vivo processing of C9046 and C9055 showed 26 and 27 nucleotide molecules as being the most abundant, and only for C9046 the 23 nucleotide molecule was the second or the third most abundant processed form.

AAV5-C9046 Induce Strong Human Intronic C9Orf72 mRNA Knockdown in to (C9Orf72 3) Line 112 (BAC-C9-112) Mice Preserving Healthy and Total Human C9Orf72 Expression.

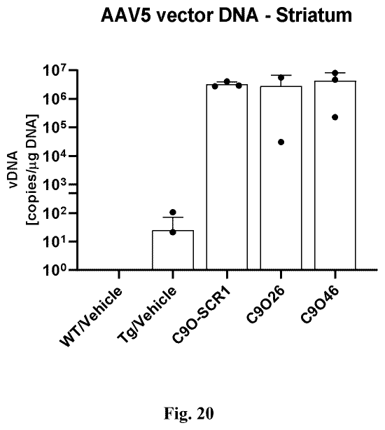

To study C9orf72 intronic mRNA lowering, C57BL/6J-Tg (C9orf72_i3) 112Lutzy/J, the Jackson Laboratory stock number: 023099 [Tg (C9orf72_3) line 112] mice were used. Tg (C9orf72_3) line 112 mice have several tandem copies of the c9orf72_3 transgene, with each copy encoding the human c9orf72 with a hexanucleotide repeat expansion in the intron between the alternatively-spliced non-coding first exons 1a and 1b. Each transgene copy has between 100-1000 G4C2 repeats. Hemizygous mice exhibit RNA foci and poly (GP) dipeptides in most neuronal populations of the brain by three months of age (https://www.jax.org/strain/023099). Intrastriatal injection of AAV5 vectors was performed in 8 weeks old female and male (n=8) mice receiving bilateral infusion of 3E+10 gc of AAV5-C9026, AAV5-C9046, and C90-SCR1 that served as negative control (FIGS. 20-24). As additional controls, a wild type mouse group and a transgenic vehicle injected group were included. Three naïve transgenic mice were sacrificed at the beginning of the in-life phase (T=0 mice) and at the end of the in-life phase (T=12 mice) to collect tissues for method development. Intrastriatal injection of AAV5 vectors is also performed in 8 weeks old female and male (n=8) mice receiving bilateral infusion of 3E+10 gc and 1.5E+11 gc of AAV5-V2S46 and AAV5-V2S48, and 1.5E+11 gc of AAV5-V2S-SCR1 and AAV5-V2SO51 (not shown). After the infusion, the mice were followed up until 20 weeks (12 weeks post-infusion) of age with weekly body weight measurements and cage side observations. At 20 weeks of age, the animals are euthanized and plasma, CSF and livers were collected from all mice and fresh-frozen. The following tissue samples were collected and fresh-frozen (n=5/group): cortex (frontal, caudal), hippocampus, striatum, thalamus, brainstem, cerebellum, hypothalamus, ventral midbrain, spinal cord C, T and L segments. Snap frozen samples were used to extract DNA for vector genome quantification and RNA for human intronic and V2 c9orf72 mRNA and miC90 quantification. Snap frozen samples are also used for V1S/V2S quantification. From 3 animals per group, the following tissues were collected and fixed: whole brain divided coronally into 4 pieces and spinal cord C, T and L segments. Fixed samples are processed for RNA foci sense and antisense and proteins immunohistochemistry (poly-GP, poly-GA, poly-GR).

Vector DNA measurements in the striatum showed few partially injected mice in the C9026 group (animal 26, 7457, 7459 considered partially dosed, final group size n=2), in the C9046 group (animal 7440 and 7441 considered partially dosed, final group size n=3) and in the C90-SCR1 group (animal 52 and 7438 considered partially dosed, final group size n=3). The partially injected animals were removed from following mRNA analyses. An average of 2.8E+6 gc/ug of DNA was measured in AAV5-C9026 injected mice, 4.3E+6 gc/ug of DNA in AAV5-C9046 and 3.2E+6 in AAV5-C90-SCR1 dosed mice (FIG. 20).

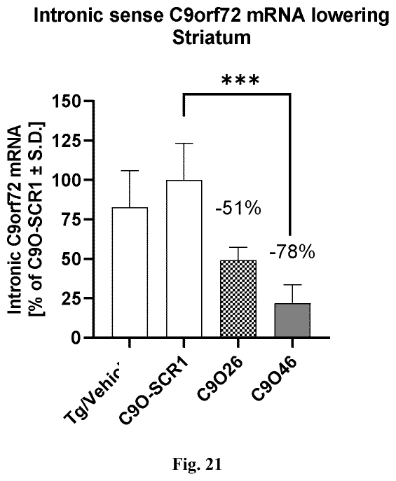

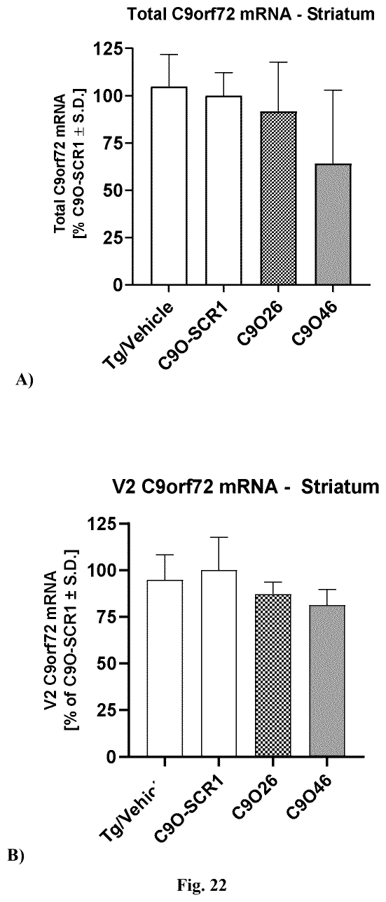

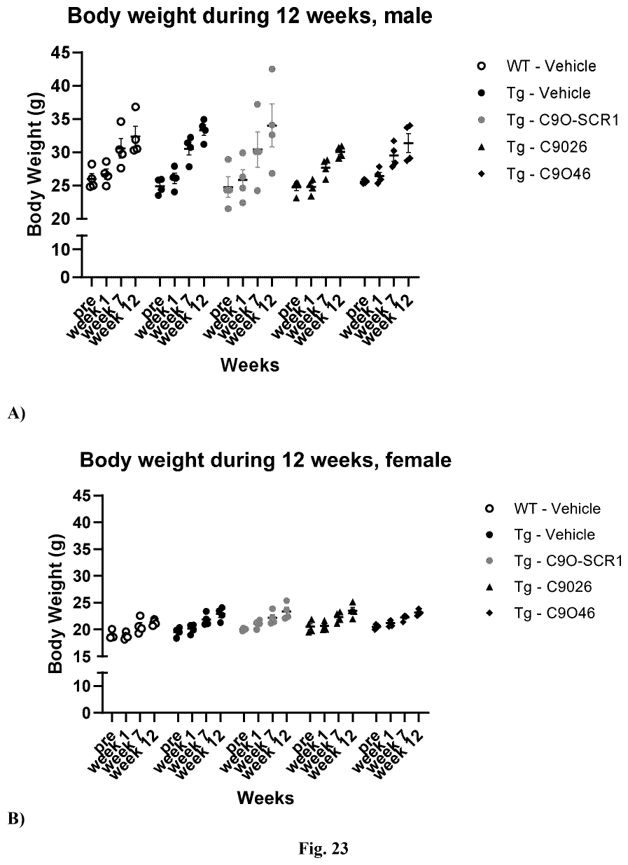

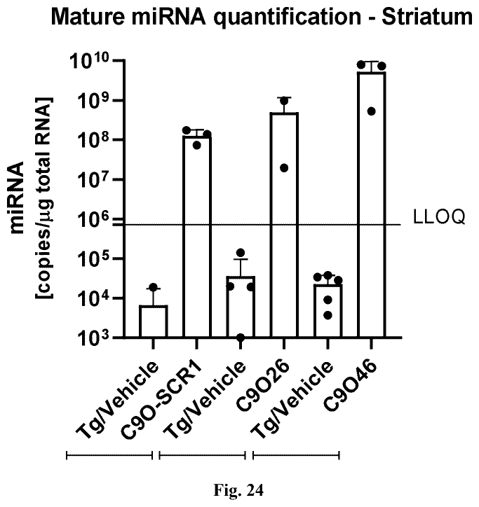

In the striatum human intronic mutant C9orf72 mRNA knockdown was observed upon AAV5-C9026 and AAV5-C9046 injection. A significant (p<0.001) decrease was achieved in C9046 group with an average decrease of approximately 77.9% compared to C90-SCR1, in C9026 approximately 50.7% intronic C9orf72 mRNA lowering was observed relative to C90-SCR1 (FIG. 21). Human total C9orf72 mRNA expression and healthy (V2) C9orf72 mRNA expression were measured in the striatum and resulted in a non-significant trend in total C9orf72 mRNA lowering in AAV5-C9046 treated mice (group average-43.1%, FIG. 22.A) and silencing of the healthy (V2) C9orf72 mRNA was not observed (FIG. 22.B). The partial total C9orf72 lowering mRNA is expected as consequence of the strong intronic mutant C9orf72 mRNA lowering. Body weight of male (FIG. 23.A) and female mice (FIG. 23.B) was measured during the 11 weeks in-life phase and normal weight gain was observed in all negative controls and AAV5 treated groups. Mature C9026, C9046 and C90-SCR1 miRNA expression levels were quantified in the striatum. An average of 5E+8 and 5.3E+9 molecules/ug of RNA were detected for AAV5-C9026 and AAV5-C9046 injected mice respectively, an average of 1.3E+8 molecules/ug of RNA was detected for AAV5-C90-SCR1 group (FIG. 24).

AAV5-C9046 and AAV5-C9055 Induce Strong Intronic Mutant C9Orf72 mRNA Knockdown in AAV9-C9Orf72 (G4C2)-149 Repeats Mice.

To study C9orf72 intronic mRNA lowering in AAV9-c9orf72 (G4C2) 149 repeats mice upon intrastriatal injection of AAV5 vectors, 5 weeks old male mice (n=6) received bilateral infusion of 3E+10 or 1.5E+11 gc of AAV5-C9026 and AAV5-C9046, and 1.5E+11 gc of AAV5-C90-SCR1 (not shown). The model was generated at The Jackson Laboratory by intracerebroventricular injection with AAV encoding 149 G4C2 repeats on postnatal day 0. The mice accumulate dipeptide repeat proteins derived from both sense and antisense transcripts and develops sense and antisense RNA foci (from 3 months of age). Additionally, the mice exhibit motor and cognitive dysfunction (6 months of age), and pathological hallmarks of ALS/FTD, including neuron loss, gliosis, aggregation of phosphorylated TDP-43, nucleocytoplasmic transport defects, accumulation of stress-granule proteins (Chew et al., 2019 Mol Neurodegener. 15;14(1):9; https://www.alzforum.org/research-models/c9orf72aavg4c2149). After the infusion, the mice were followed up until 13 weeks (8 weeks post-infusion) of age with weekly body weight measurements and cage side observations. At 8 weeks post-infusion, the animals were euthanized and plasma, CSF and liver were collected from all mice and fresh-frozen. The following tissue samples were collected and fresh-frozen: cortex (frontal, caudal), hippocampus, striatum, thalamus, brainstem, cerebellum, hypothalamus, ventral midbrain, spinal cord C, T and L segments. Snap frozen samples were used to extract DNA for vector genome quantification and RNA for human intronic C9orf72 mRNA and miC90 quantification. Snap frozen samples are also used for V2 C9orf72 mRNA or V1S/V2S quantification.

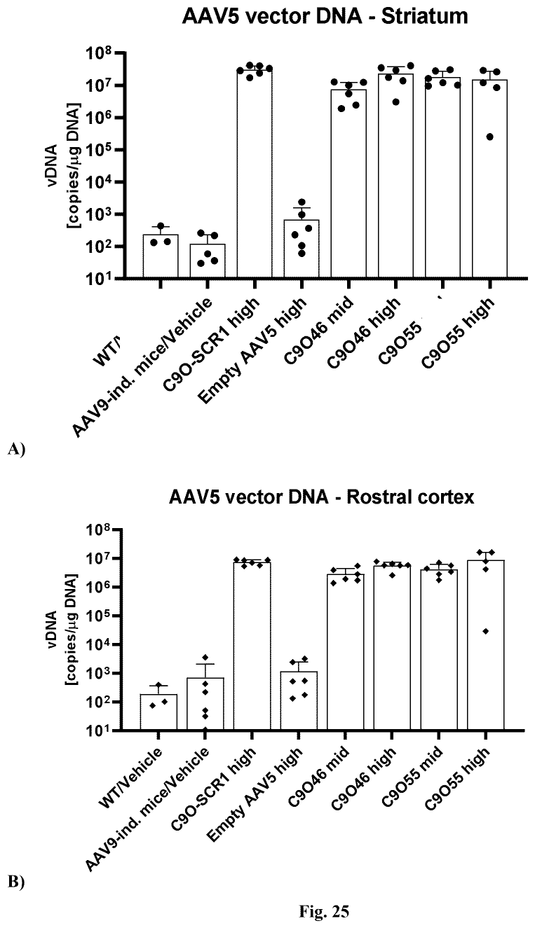

Vector DNA measurements in the striatum and rostral cortex showed that there was one mouse partially injected in the C9055 high dose group (animal #35, final group size n=5). This animal was excluded from following mRNA analyses. In the striatum an average of 7.4E+6 gc/ug of DNA was measured in the C9046 mid dose group, 1.8E+7 gc/ug of DNA in the C9055 mid dose group, and 2.3E+7, 1.52E+7 and 3.1E+7 gc/ug of DNA respectively in C9046, C9055 and C90-SCR1 high dose groups (FIG. 25.A). In the rostral cortex an average of 2.9E+6 gc/ug of DNA was measured in C9046 mid dose group, 4.2E+6 gc/ug of DNA in C9055 mid dose and 5.6E+6, 8.9E+6 and 7.4E+6 gc/ug of DNA respectively in C9046, C9055 and C90-SCR1 high dose (FIG. 25.B).

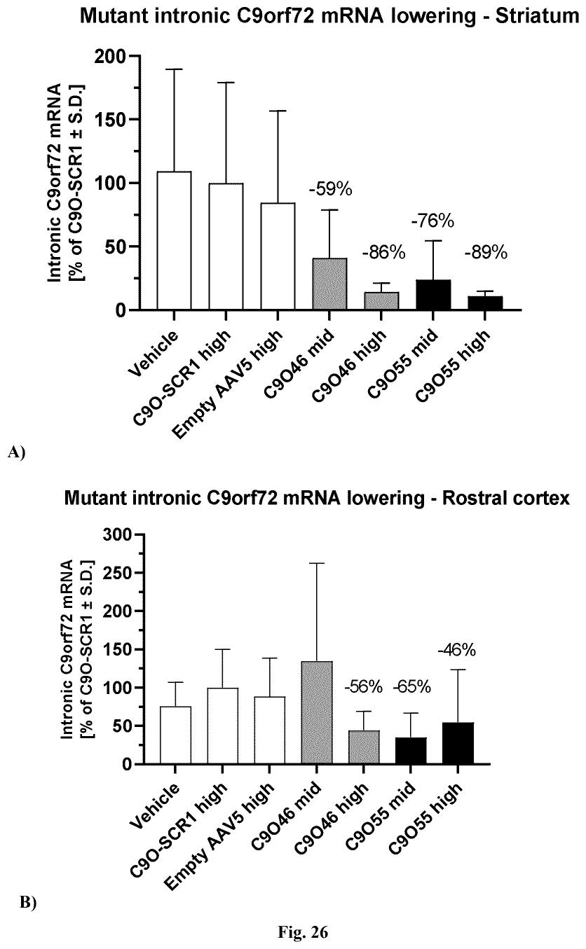

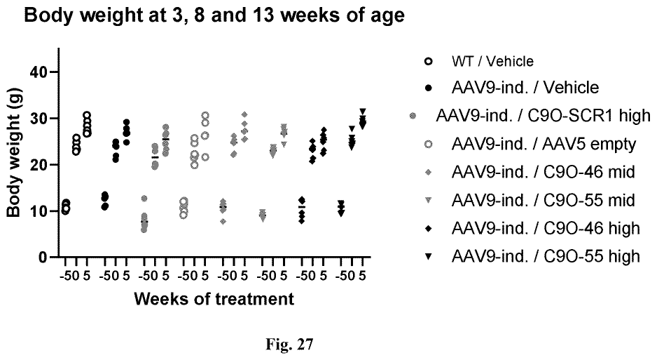

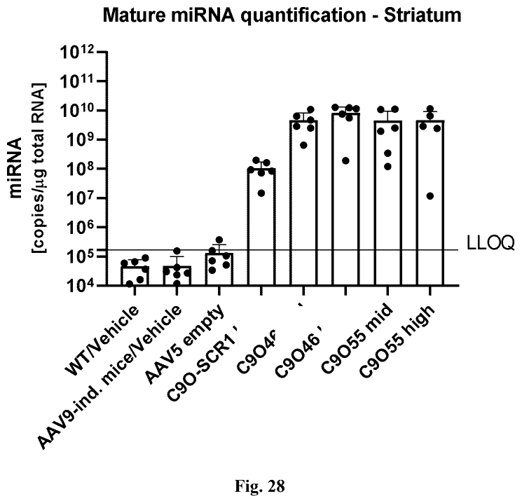

In the striatum a dose-dependent decrease in human intronic mutant C9orf72 mRNA expression was observed upon AAV5-C9046 and AAV5-C9055 injection. A strong decrease was achieved at high dose with an average mutant intronic C9orf72 mRNA lowering of approximately 85.7% (C9046)-88.8% (C9055) compared to the C90-SCR1 group, at mid dose the measured intronic mutant C9orf72 lowering was approximately 59% (C9046)-76.1% (C9055) compared to C90-SCR1 and reaching significance for C9055 (p<0.05) (FIG. 26.A). In the rostral cortex a trend in decrease of human intronic mutant C9orf72 mRNA expression was observed upon AAV5-C9046 and AAV5-C9055 injection. An average decrease of approximately 45.5% (C9055)-56% (C9046) compared to the C90-SCR1 was observed at high dose, and for C9055 mid dose an average of 64.9% knockdown was observed (FIG. 26.B). The slightly higher potency at mid dose of C9055 compared to C9046 in striatum and rostral cortex could be explained by the higher number of detected vector DNA copies (2.4 times). Body weight of the mice was measured during the 8 weeks in-life phase and normal weight gain was observed in all negative controls and AAV5 treated groups (FIG. 27). Mature C9046, C9055 and C90-SCR1expression levels were quantified in the striatum tissue samples. An average of 4.6E+9 and 4.5E+9 molecules/ug of RNA was detected for C9046 and C9055 mid dose groups, an average of 8.3E+9 and 4.7E+9 molecules/ug of RNA ware detected for C9046 and C9055 high dose, respectively (FIG. 28).

AAV5-C9046 and AAV5-C9055 Induce Strong Mutant Intronic C9Orf72 mRNA Knockdown and Improve ALS Pathological Markers in AAV9-C9Orf72 (G4C2)-149 Repeats Mice at 18 Weeks Post-Infusion.

To provide Proof of Concept for C90 and study whether the lowering of intronic C9orf72 mRNA upon C90 overexpression resulted in phenotypic improvement in AAV9-C9orf72 (G4C2) 149-induced mice upon intrastriatal injection of AAV5 vectors, 8 weeks old male mice (n=7-16) receive bilateral infusion of 3E+10 or 1.5E+11 gc of AAV5-C9055 and AAV5-C9046, and 1.5E+11 gc of AAV5-C90-SCR1 and empty AAV5. After the infusion, the mice were followed up for 18 weeks with weekly body weight measurements and cage side observations. Following the behavioral tests, at 18 weeks post-infusion the animals were euthanized and plasma, and liver were collected from all mice and fresh-frozen. For the animal dedicated to molecular analysis (n=4-10), the following tissue samples were collected and fresh-frozen: cortex (frontal, caudal), hippocampus, striatum, thalamus, brainstem, cerebellum, hypothalamus, ventral midbrain, spinal cord C, T and L segments. Snap frozen samples were used to extract DNA for vector genome quantification and RNA for human intronic C9orf72 mRNA and miC90 quantification. Snap frozen samples are also used for V2 C9orf72 mRNA or V1S/V2S quantification. For animals dedicated to histological readouts (n=3-6) the whole brain was fixed and embedded in paraffin.

Vector DNA measurements in the striatum highlighted seven potential outlier mice based on the vector DNA levels of the group (animals #91, #125, #126, #180, #226, #228 and #232). The potential outlier animals were excluded from following additional analyses.

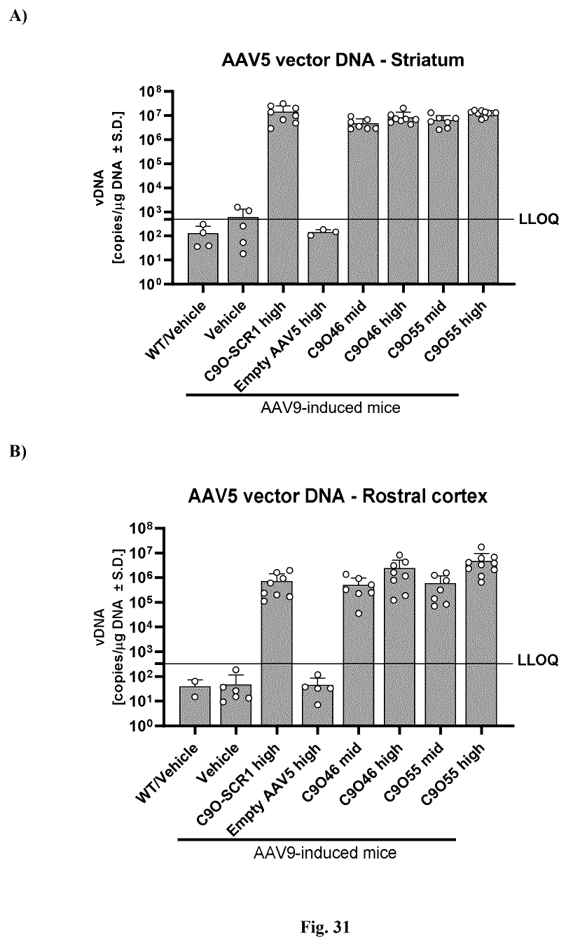

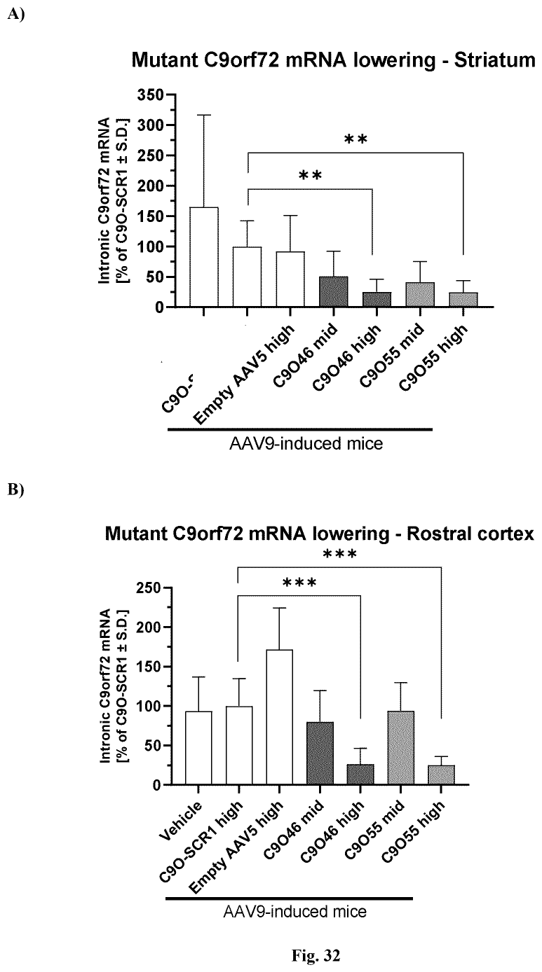

In the striatum an average of 4.8E+6 GC/ug of DNA was measured in the C9046 mid dose group, 6.4E+6 GC/ug of DNA in the C9055 mid dose group, and 8.4E+6, 1.2E+7 and 1.4E+7 GC/ug of DNA respectively in C9046, C9055 and C90-SCR1 high dose groups (FIG. 31.A). In the rostral cortex an average of 5.2E+5 GC/ug of DNA was measured in C9046 mid dose group, 6E+5 GC/ug of DNA in C9055 mid dose and 2.5E+6, 4.8E+6 and 7.4E+5 GC/ug of DNA respectively in C9046, C9055 and C90-SCR1 high dose (FIG. 31.B). In the striatum a dose-dependent decrease in human mutant intronic C9orf72 mRNA expression was observed upon AAV5-C9046 and AAV5-C9055 injection. A significant strong decrease was achieved at high dose with an average mutant intronic C9orf72 mRNA lowering of approximately 75% (C9046, p<0.01)—76% (C9055, p<0.01) compared to the C90-SCR1 group, at mid dose the measured intronic mutant C9orf72 lowering was between 49.1% (C9046)-58.9% (C9055) compared to C90-SCR1 (FIG. 32.A). In the rostral cortex also a significant decrease of human intronic mutant C9orf72 mRNA expression was observed upon injection of AAV5-C9046 and AAV5-C9055 at high dose, a less pronounced effect was observed in rostral cortex compared to the injection site striatum due to lower AAV biodistribution. An average decrease of approximately 73.7% (C9046, p<0.001)-75.2% (C9055, p<0.001) compared to the C90-SCR1 was observed (FIG. 32.B).

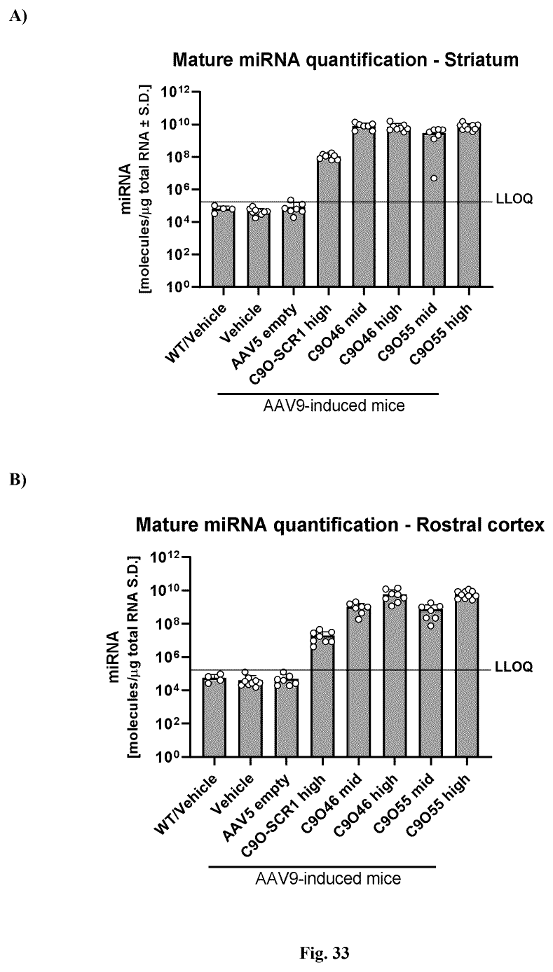

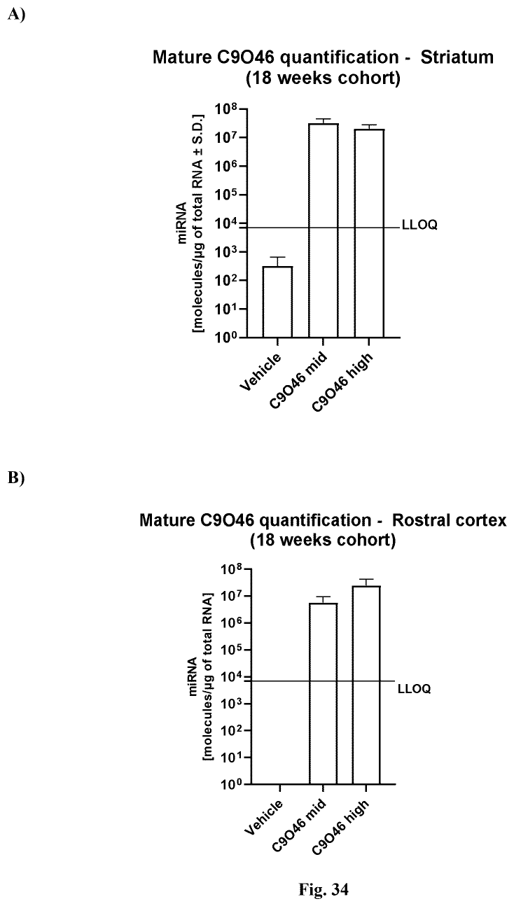

Mature C9046, C9055 and C90-SCR1 expression levels were quantified in the striatum and rostral cortex tissue samples with RT-QPCR SYBR green assays. An average of 8.3E+9 and 3E+9 molecules/ug of RNA was detected for C9046 and C9055 mid dose groups, an average of 7.2E+9, 7.5E+9 and 1.1E+8 molecules/ug of RNA was measured respectively in C9046, C9055 and C90-SCR1 high dose groups (FIG. 33.A) in the striatum. An average of 1E+9 and 7.3E+8 molecules/ug of RNA was detected for C9046 and C9055 mid dose groups, an average of 6E+9, 6.1E+9 and 1.9E+7 molecules/ug of RNA respectively in C9046, C9055 and C90-SCR1 high dose (FIG. 33.B) in the rostral cortex.

A TaqMan miRNA assay was designed to quantify the most abundant processed isoform of C9046 (26 nucleotides) previously identified with small RNA-seq in mouse brain (FIG. 19.B). Overall, the analysis with the isoform specific TaqMan miRNA assay resulted in lower molecules/ug quantified due to reduced assay background and specificity in the quantified isoform. In striatum an average of 3.2E+7 and 2E+7 molecules/ug of RNA for C9046 mid and high dose respectively were quantified (FIG. 34.A). In rostral cortex an average of 5.7E+6 and 2.4E+7 molecules/ug of RNA for C9046 mid and high dose respectively were quantified (FIG. 34.B).

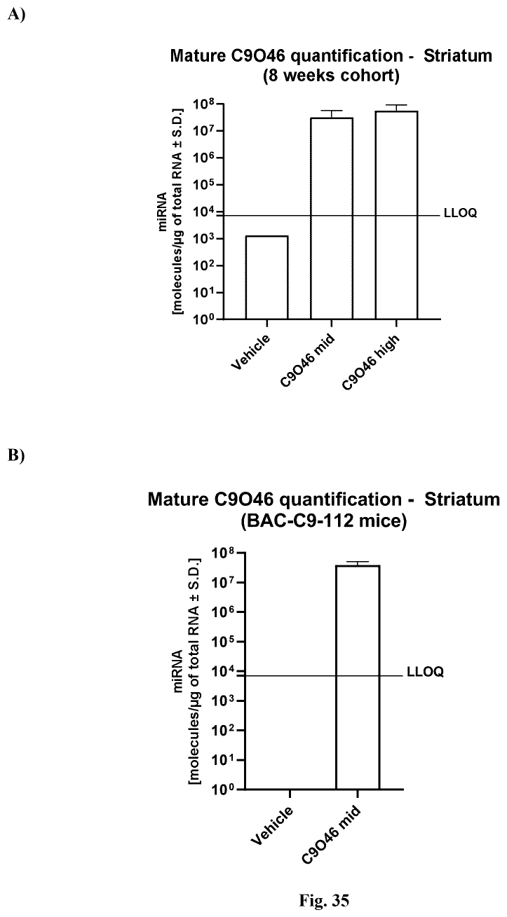

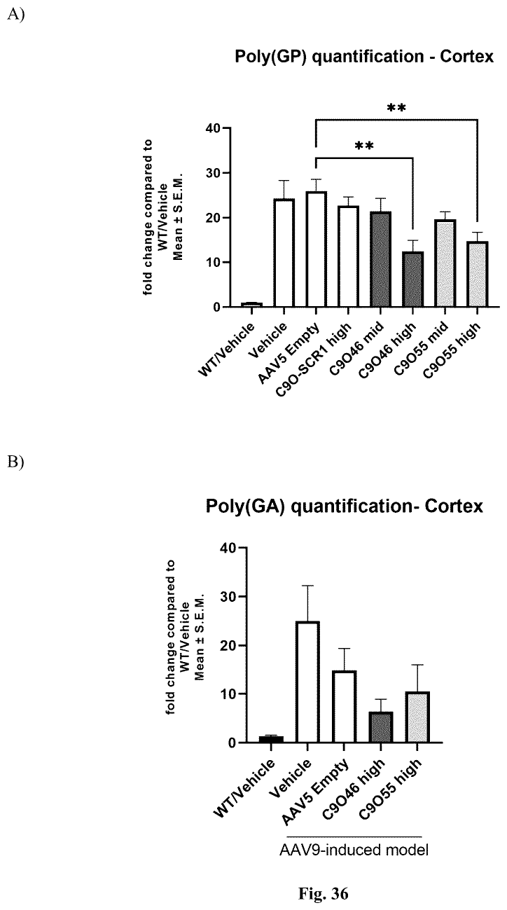

Additionally, the C9046 striatum samples from the 8 weeks post-infusion cohort [AAV9-C9orf72 (G4C2)-149 repeats mice] and the striatum samples of transgenic BAC-C9-112 mice dosed with C9046 were analyzed with the newly designed TaqMan miRNA assay. In striatum samples of the 8 weeks post-infusion cohort showed 3.2E+7 and 5.7E+7 molecules/ug of RNA for C9046 mid and high dose respectively (FIG. 35.A). In striatum of BAC-C9-112 samples 3.9E+7 molecules/ug of RNA were detected for C9046 mid dose (FIG. 35.B).

The AAV9-C9orf72 (G4C2) 149 repeats mice accumulate dipeptide repeat proteins (DPRs) [poly (GP), poly (GA), poly (GR)] derived from both sense and antisense transcripts and develops sense and antisense RNA foci (from 3 months of age). Additionally, the mice exhibit motor and cognitive dysfunction (6 months of age), and pathological hallmarks of ALS/FTD, including RNA foci sense and antisense. To investigate if intronic C9orf72 mRNA lowering corresponded to decreased levels of poly (GP) and, in a subset of high dose samples (n=4-6) due to low material availability, poly (GA). Two MSD assays were performed on the hemibrain cortices collected from the animals at 18 weeks post-injection, the soluble fraction of the lysates was analyzed. The results showed that treatment with a high dose of AAV5-C9046 and AAV5-C9055 significantly decreased levels of soluble poly (GP) compared to AAV9-induced mice injected with empty AAV5 (p<0.01) group (FIG. 36.A). The results showed a more evident trend in decreased poly (GA) in C9046 high dose group compared to C9055 high dose group (FIG. 36.B)

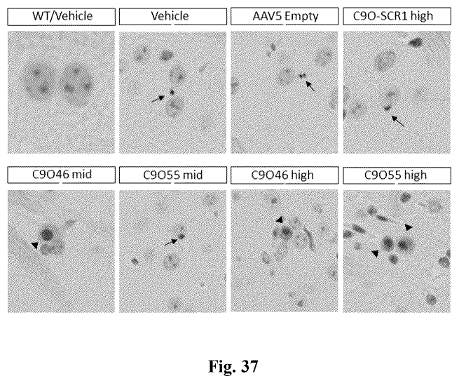

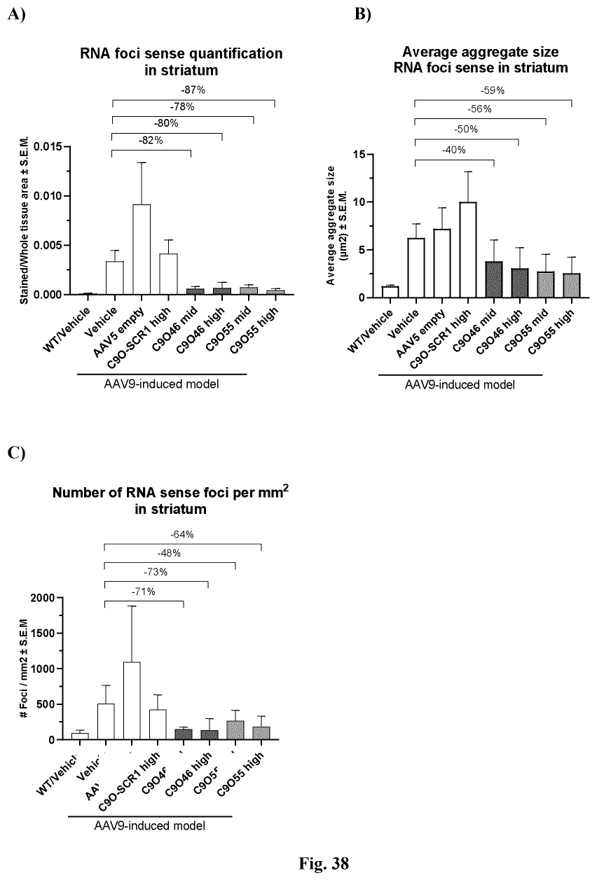

Striatum and cortex were analyzed for cytoplasmic aggregates for poly (GA) in 3-6 mice per group using immunohistochemistry (IHC) analyses. Mice treated with C9046 (mid and high dose) and C9055 (high dose) showed a trend in increase of cells with a different poly (GA) staining pattern. As shown in FIG. 37, while AAV9 induced controls had round, perinuclear poly (GA) aggregates (indicated by arrows), treated mice showed cells with a diffused poly (GA) staining, which filled the entire cytoplasm (indicated by arrowheads). Furthermore, IHC will be used to analyze striatum and cortex for cytoplasmic aggregates of poly (GP) and poly (GR).

Striatum and cortex were analyzed for sense and antisense RNA foci in 3-6 mice per group for chromogenic in situ hybridization (ISH) analyses. The parameters analyzed were: stained area/whole area, average aggregate size (μm2) and Number of RNA foci/mm2. Overall, the levels of RNA foci were higher in the cortex compared to striatum, and sense RNA foci were detected at higher level compared to antisense RNA foci (in relative order: sense RNA foci cortex>sense RNA foci striatum >antisense RNA foci cortex>antisense RNA foci striatum). The effect of treatment was more pronounced in striatum, the injection site, compared to cortex in which a lower AAV biodistribution was expected. In striatum a trend toward decreased stained area ratio of sense RNA foci was observed for C9046 mid and high dose (˜80%-82%) and C9055 mid and high dose (˜78%-87%) when compared to AAV9-induced mice injected with vehicle (FIG. 38.A). In striatum treatment with C9046 mid dose and high dose showed a decrease in average aggregate size respectively of ˜40% and ˜50% when compared to AAV9-induced mice treated with vehicle (FIG. 38.B). Treatment with C9055 mid dose showed a decrease in average aggregate size of ˜56% when compared to AAV9-induced mice treated with vehicle, and ˜59% decrease in C9055 high dose injected group (FIG. 38.B). In the striatum a trend toward decreased number of sense RNA foci was observed for AAV5-C9046 and AAV5-C9055 treatment groups (mid and high dose) when compared to AAV9-induced mice injected with vehicle, between ˜71%-˜ 73% for C9046 treated groups and between ˜48%-˜ 64% for C9055 treated groups (FIG. 38.C).

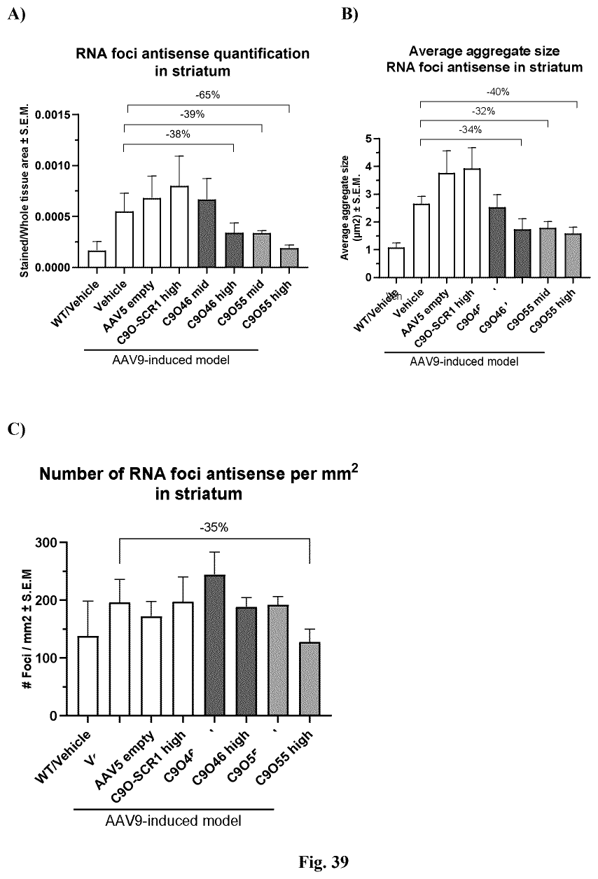

In striatum a trend toward decreased stained area ratio of antisense RNA foci was observed for AAV5-C9046 high dose (˜38%) and AAV5-C9055 mid and high dose (˜39-˜ 65%) treatment groups when compared to AAV9-induced mice injected with vehicle (FIG. 39.A). In striatum C9046 high dose and C9055 mid and high dose showed a trend toward decrease in average aggregate size of antisense RNA foci was measured, respectively of ˜34%, ˜32% and ˜40% when compared to AAV9-induced mice treated with vehicle (FIG. 39.B). In striatum a mild trend toward decreased number of antisense RNA foci was observed for C9055 high dose group (˜35%) compared to AAV9-induced mice vehicle treated (FIG. 39.C).

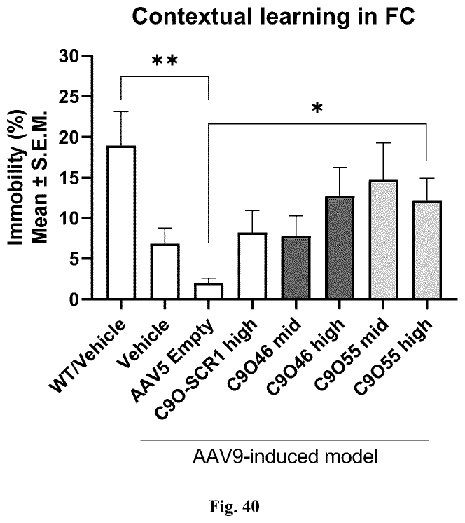

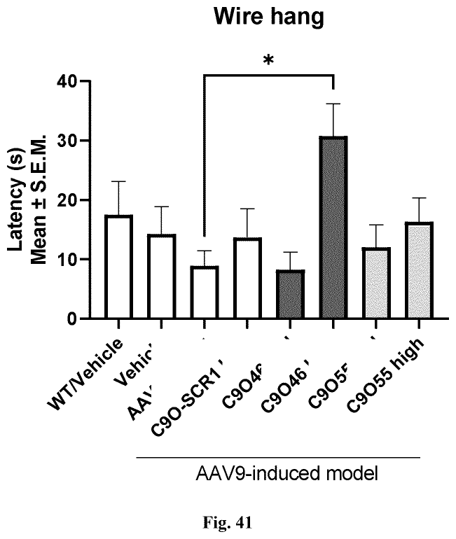

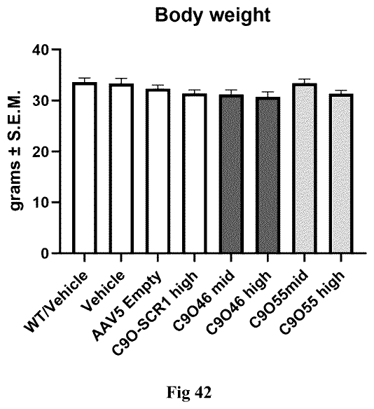

At week 18 post-infusion, behavioral assessment was performed using the fear conditioning test and the wire hang test in all the AAV9-C9orf72 (G4C2)-149 repeats mice enrolled in the study to assess the impact of treatment on cognitive and motor function. The empty AAV5 group showed significantly lower percentage of immobility (p<0.01) compared to WT/Vehicle treated group during exposure to the context (FIG. 40) and was selected as the negative control group, however the AAV9-induced model injected with vehicle did not show expected impairment of contextual learning. The results obtained with C9046 high dose treatment and C9055 mid and high dose treatments suggested rescued impaired contextual learning in the fear conditioning when compared to the empty AAV5 group (in average respectively, 10.81%, 12.74% and 10.27% increase of immobility), statistical significance was reached for C9055 high dose (p<0.05) (FIG. 40). The results of the wire hang test showed that the C9046 high dose group had a significantly (p<0.05) increased latency time compared to empty AAV5 group (average latency time of C9046 was 30.76 seconds versus 8.91 seconds of empty AAV5 group; FIG. 41), suggesting an effect of C9046 treatment in retaining muscle strength. In the study the body weight and general health were monitored, no differences in the mice growth were found throughout the duration of the study (week 4 until week 26 (FIG. 42).

### Non-Human Primate Pilot Study.

The objective of this study is to evaluate the toxicity and biodistribution of AAV-C9046 when administered as a single dose via intrathecal (IT) infusion to the cynomolgus monkey. The test material will be administered at three dosing levels along the spinal cord via an IT catheter introduced by a lumbar puncture. After dosing, animals are observed post dose for 4 weeks to assess the reversibility or persistence of any effects. In addition, an objective is to provide data for the dose selection of AAV-C9046 for a consecutive subsequent GLP toxicity study in cynomolgus monkeys and to investigate which final AAV vector candidate (AAV5 or AAV9) gives the best most appropriate biodistribution and safety profile.

Proof-of-Concept study in mice. To provide Proof of Concept for C90 and study whether the lowering of intronic C9orf72 mRNA results in phenotypic improvement in AAV9-C9orf72 (G4C2)-149 repeats mice upon intrastriatal injection of AAV5 and AAV9 vectors, 7 weeks old male mice (n=12) and female (n=12) mice will receive bilateral infusion of 1.8E+12 GC of AAV5-C9046 and 1.2E+12 GC AAV9. At six months of age, behavioral tests are performed with open field test, contextual fear conditioning, wire hang, grip strength and clasping. At nine months of age open field test, wire hang, grip strength and clasping behavioral tests are performed. At the end of behavioral tests the animals are euthanized by CO2 narcosis and plasma, CSF and liver are collected from all mice and fresh-frozen. The following tissue samples are collected and fresh-frozen: cortex (frontal, caudal), hippocampus, striatum, thalamus, brainstem, cerebellum, hypothalamus, ventral midbrain, spinal cord C, T and L segments. Snap frozen samples are used to extract DNA for vector genome quantification, RNA for human intronic C9orf72 mRNA and miC90 quantification, and poly (GP) and poly (GA) quantification with MSD. From a subset of animals per group, the following tissues are collected and fixed: whole brain divided coronally into 4 pieces and spinal cord C, T and L segments. Fixed samples are analyzed for sense and antisense RNA foci, DPRs aggregates detection [poly (GP), poly (GA), poly (GR)].

Dose response study in mice. The aim of the study is to provide relevant information for the dose translation to large animals and eventually the clinic based on the dose dependent lowering of intronic C9orf72 mRNA upon AAV-C9046 injection. To obtain dose dependent lowering of C9orf72 intronic mRNA, C57BL/6J-Tg (C9orf72_i3) 112Lutzy/J, the Jackson Laboratory stock number: 023099 [Tg (C9orf72_3) line 112] 10-12 weeks male and female mice are used (n=20-24). Tg (C9orf72_3) line 112 mice have several tandem copies of the c9orf72_3 transgene, with each copy encoding the human c9orf72 with a hexanucleotide repeat expansion in the intron between the alternatively-spliced non-coding first exons 1a and 1b. Each transgene copy has between 100-1000 G4C2 repeats. Hemizygous mice exhibit RNA foci and poly (GP) dipeptides in most neuronal populations of the brain by three months of age. The mice receive bilateral infusion of 1.8E+12 GC, 3.6E+11 GC, 7.2E+10 GC and 1.4E+10 GC of AAV-C9046, vehicle will be used as negative control. Eight to twelve weeks post-dosing the animals are euthanized and plasma, CSF, liver and spleen are collected from all mice and fresh-frozen. The following tissue samples are collected and fresh-frozen: cortex (frontal, caudal), hippocampus, striatum, thalamus, brainstem, cerebellum, hypothalamus, ventral midbrain, spinal cord C, T and L segments. Snap frozen samples are used to extract DNA for vector genome quantification, RNA for human intronic C9orf72 mRNA, V2 C9orf72 mRNA and C90 quantification.

1. A nucleic acid comprising a sequence encoding a first RNA and a sequence encoding a second RNA, wherein:
i. the first and second RNA each comprise a hairpin;
ii. the second RNA comprises a guide sequence of at least 19 nucleotides substantially complementary to part of the c9orf72 gene; and
iii. the C9orf72 gene comprises a GGGGCC (G4C2) hexanucleotide repeat expansion of at least 24 repeats in a non-coding region.

2. The nucleic acid according to claim 1, wherein in a 5′ to 3′ direction, the sequence encoding the first RNA is followed by a first spacer comprising at least 15 nucleotides, which first spacer is followed by the sequence encoding the second RNA.

3. The nucleic acid according to claim 1, wherein the second RNA comprises SEQ ID NO. 229 or a variant thereof.

4. The nucleic acid according to claim 1, wherein the first RNA comprises a sequence selected from the group consisting of: SEQ ID NO. 181 or a variant thereof; SEQ ID NO. 229 or a variant thereof; and SEQ ID NO. 230 or a variant thereof.

5. The nucleic acid according to claim 4, wherein the first RNA comprises SEQ ID NO. 181 or a variant thereof.

6. The nucleic acid according to claim 5, wherein the first RNA is mutated to reduce or eliminate miR-144 processing and expression of the first RNA.

7. The nucleic acid according to claim 5, further comprising a sequence encoding a third RNA, and wherein, preferably, in a 5′ to 3′ direction, the sequence encoding the second RNA is followed by a second spacer comprising at least 15 nucleotides, which second spacer is followed by the sequence encoding the third RNA.

8. The nucleic acid according to claim 7, wherein the third RNA comprises a hairpin and a guide sequence of at least 19 nucleotides substantially complementary to part of the c9orf72 gene, wherein the C9orf72 gene comprises a G4C2 hexanucleotide repeat expansion of at least 24 repeats in a non-coding region.

9. The nucleic acid according to claim 8, wherein the third RNA comprises SEQ ID NO. 229 or a variant thereof.

10. The nucleic acid according to claim 1, wherein the first RNA comprises: SEQ ID NO. 230 or a variant thereof; and a guide sequence of at least 19 nucleotides substantially complementary to a target sequence in a transcript encoded by the C9orf72 gene; wherein the c9orf72 gene comprises a G4C2 hexanucleotide repeat expansion in a non-coding region.

11. The nucleic acid according to claim 1, wherein each of the guide sequences is selected selected from the group consisting of: SEQ ID NO. 1 to SEQ ID NO. 68.

12. Nucleic acid according to claim 1, wherein the at least one target sequence is comprised in an antisense RNA transcript encoded by the human C9orf72 gene.

13. An expression cassette comprising the nucleic acid according to claim 1, wherein the expression cassette is a DNA molecule.

14. The expression cassette according to claim 13, wherein the nucleic acid is operably linked to a promoter and optionally to a poly-A signal.

15. The expression cassette according to claim 13, wherein the expression cassette is flanked by at least one AAV Inverted Terminal Repeat (ITR).

16. An adeno-associated virus (AAV) vector comprising the expression cassette according to claim 13.

17. The AAV vector according to claim 16 comprising an AAV5 or AAV9 capsid protein.

18. A pharmaceutical composition comprising the nucleic acid according to claim 1, and at least one pharmaceutically acceptable excipient.

19. The nucleic acid according to claim 1 for use as a medicament or in a treatment for reducing expression of RNA encoded by the human C9orf72 gene.

20. (canceled)

21. A method of treating and/or preventing amyotrophic lateral sclerosis (ALS) and/or frontotemporal dementia (FTD), or any atypical presentations mimicking other kinds of neurodegenerative brain diseases, the method comprising administering the nucleic acid according to claim 1.

22. A kit comprising a nucleic acid according to claim 1 and an immunosuppressive compound.

23. A cell comprising the nucleic acid according to claim 1.


SEQUENCE LISTING (263 sequences)

SEQ ID NO: 1 - DNA, 1950 bp, Homo sapiens
  source 1..1950: mol_type = other DNA
acgtaacctacggtgtcccgctaggaaagagaggtgcgtcaaacagcgacaagttccgcccacgtaaaagatgacgcttgatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcaagtaagaatttttcttttcataaaagctggatgaagcagataccatcttatgctcacctatgacaagatttggaagaaagaaaataacagactgtctacttagattgttctagggacattacgtatttgaactgttgcttaaatttgtgttatttttcactcattatatttctatatatatttggtgttattccatttgctatttaaagaaaccgagtttccatcccagacaagaaatcatggccccttgcttgattctggtttcttgttttacttctcattaaagctaacagaatcctttcatattaagttgtactgtagatgaacttaagttatttaggcgtagaacaaaattattcatatttatactgatctttttccatccagcagtggagtttagtacttaagagtttgtgcccttaaaccagactccctggattaatgctgtgtacccgtgggcaaggtgcctgaattctctatacacctatttcctcatctgtaaaatggcaataatagtaatagtacctaatgtgtagggttgttataagcattgagtaagataaataatataaagcacttagaacagtgcctggaacataaaaacacttaataatagctcatagctaacatttcctatttacatttcttctagaaatagccagtatttgttgagtgcctacatgttagttcctttactagttgctttacatgtattatcttatattctgttttaaagtttcttcacagttacagattttcatgaaattttacttttaataaaagagaagtaaaagtataaagtattcacttttatgttcacagtcttttcctttaggctcatgatggagtatcagaggcatgagtgtgtttaacctaagagccttaatggcttgaatcagaagcactttagtcctgtatctgttcagtgtcagcctttcatacatcattttaaatcccatttgactttaagtaagtcacttaatctctctacatgtcaatttcttcagctataaaatgatggtatttcaataaataaatacattaattaaatgatattatactgactaattgggctgttttaaggctcaataagaaaatttctgtgaaaggtctctagaaaatgtaggttcctatacaaataaaagataacattgtgcttata

SEQ ID NO: 2 - DNA, 3338 bp, Homo sapiens
  source 1..3338: mol_type = other DNA
acgtaacctacggtgtcccgctaggaaagagaggtgcgtcaaacagcgacaagttccgcccacgtaaaagatgacgcttggtgtgtcagccgtccctgctgcccggttgcttctcttttgggggcggggtctagcaagagcaggtgtgggtttaggagatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcaatgccatcagctcacacttgcaaacctgtggctgttccgttgtagtaggtagcagtgcagagaaagtaaataagatagtcagaacattatgcctttttctgactccagcagagagaaaatgctccaggttatgtgaagcagaatcatcatttaaatatgagtcagggctctttgtacaaggcctgctaaaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgaatatttttcaagatgtcttacacagagacactctagtgaaagccttcctggatcaggtctttcagctgaaacctggcttatctctcagaagtactttccttgcacagtttctacttgtccttcacagaaaagccttgacactaataaaatatatagaagacgatacgcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgactttttaaatgtgtaacttaataagcctattccatcacaatcatgatcgctggtaaagtagctcagtggtgtggggaaacgttcccctggatcatactccagaattctgctctcagcaattgcagttaagtaagttacactacagttctcacaagagcctgtgaggggatgtcaggtgcatcattacattgggtgtctcttttcctagatttatgcttttgggatacagacctatgtttacaatataataaatattattgctatcttttaaagatataataataggatgtaaacttgaccacaactactgtttttttgaaatacatgattcatggtttacatgtgtcaaggtgaaatctgagttggcttttacagatagttgactttctatcttttggcattctttggtgtgtagaattactgtaatacttctgcaatcaactgaaaactagagcctttaaatgatttcaattccacagaaagaaagtgagcttgaacataggatgagctttagaaagaaaattgatcaagcagatgtttaattggaattgattattagatcctactttgtggatttagtccctgggattcagtctgtagaaatgtctaatagttctctatagtccttgttcctggtgaaccacagttagggtgttttgtttattttattgttcttgctattgttgatattctatgtagttgagctctgtaaaaggaaattgtattttatgttttagtaattgttgccaactttttaaattaattttcattatttttgagccaaattgaaatgtgcacctcctgtgccttttttctccttagaaaatctaattacttggaacaagttcagatttcactggtcagtcattttcatcttgttttcttcttgctaagtcttaccatgtacctgctttggcaatcattgcaactctgagattataaaatgccttagagaatatactaactaataagatctttttttcagaaacagaaaatagttccttgagtacttccttcttgcatttctgcctatgtttttgaagttgttgctgtttgcctgcaataggctataaggaatagcaggagaaattttactgaagtgctgttttcctaggtgctactttggcagagctaagttatcttttgttttcttaatgcgtttggaccattttgctggctataaaataactgattaatataattctaacacaatgttgacattgtagttacacaaacacaaataaatattttatttaaaattctggaagtaatataaaagggaaaatatatttataagaaagggataaaggtaatagagcccttctgccccccacccaccaaatttacacaacaaaatgacatgttcgaatgtgaaaggtcataatagctttcccatcatgaatcagaaagatgtggacagcttgatgttttagacaaccactgaactagatgactgttgtactgtagctcagtcatttaaaaaatatataaatactaccttgtagtgtcccatactgtgttttttacatggtagattcttatttaagtgctaactggttattttctttggctggtttattgtactgttatacagaatgtaagttgtacagtgaaataagttattaaagcatgtgtaaacattgttatatatcttttctcctaaatggagaattttgaataaaatatatttgaaatttt

SEQ ID NO: 3 - DNA, 164 bp, Homo sapiens
  source 1..164: mol_type = other DNA
gtgtgtgtttttgtttttcccaccctctctccccactacttgctctcacagtactcgctgagggtgaacaagaaaagacctgataaagattaaccagaagaaaacaaggagggaaacaaccgcagcctgtagcaagctctggaactcaggagtcgcgcgctagg

SEQ ID NO: 4 - RNA, 22 nt, synthetic construct
  source 1..22: mol_type = other RNA
ctagcgggacaccgtaggttac

SEQ ID NO: 5 - RNA, 22 nt, synthetic construct
  misc_feature 1..22: note = strands RNA
  source 1..22: mol_type = other RNA
tcctagcgggacaccgtaggtt

SEQ ID NO: 6 - RNA, 22 nt, synthetic construct
  misc_feature 1..22: note = strands RNA
  source 1..22: mol_type = other RNA
acgcacctctctttcctagcgg

SEQ ID NO: 7 - RNA, 22 nt, synthetic construct
  misc_feature 1..22: note = strands RNA
  source 1..22: mol_type = other RNA
tgacgcacctctctttcctagc

SEQ ID NO: 8 - RNA, 22 nt, synthetic construct
  misc_feature 1..22: note = strands RNA
  source 1..22: mol_type = other RNA
ttgacgcacctctctttcctag

SEQ ID NO: 9 - RNA, 22 nt, synthetic construct
  misc_feature 1..22: note = strands RNA
  source 1..22: mol_type = other RNA
tttgacgcacctctctttccta

SEQ ID NO: 10 - RNA, 22 nt, synthetic construct
  misc_feature 1..22: note = strands RNA
  source 1..22: mol_type = other RNA
tcgctgtttgacgcacctctct

SEQ ID NO: 11 - RNA, 22 nt, synthetic construct
  misc_feature 1..22: note = strands RNA
  source 1..22: mol_type = other RNA
tgtcgctgtttgacgcacctct

SEQ ID NO: 12 - RNA, 22 nt, synthetic construct
  misc_feature 1..22: note = strands RNA
  source 1..22: mol_type = other RNA
ttgtcgctgtttgacgcacctc

SEQ ID NO: 13 - RNA, 22 nt, synthetic construct
  misc_feature 1..22: note = strands RNA
  source 1..22: mol_type = other RNA
acttgtcgctgtttgacgcacc

SEQ ID NO: 14 - RNA, 22 nt, synthetic construct
  misc_feature 1..22: note = strands RNA
  source 1..22: mol_type = other RNA
tacgtgggcggaacttgtcgct

SEQ ID NO: 15 - RNA, 22 nt, synthetic construct
  misc_feature 1..22: note = strands RNA
  source 1..22: mol_type = other RNA
ttacgtgggcggaacttgtcgc

SEQ ID NO: 16 - RNA, 22 nt, synthetic construct
  misc_feature 1..22: note = strands RNA
  source 1..22: mol_type = other RNA
tttacgtgggcggaacttgtcg

SEQ ID NO: 17 - RNA, 22 nt, synthetic construct
  misc_feature 1..22: note = strands RNA
  source 1..22: mol_type = other RNA
ttttacgtgggcggaacttgtc

SEQ ID NO: 18 - RNA, 22 nt, synthetic construct
  misc_feature 1..22: note = strands RNA
  source 1..22: mol_type = other RNA
agcgtcatcttttacgtgggcg

SEQ ID NO: 19 - RNA, 22 nt, synthetic construct
  misc_feature 1..22: note = strands RNA
  source 1..22: mol_type = other RNA
aagcgtcatcttttacgtgggc

SEQ ID NO: 20 - RNA, 22 nt, synthetic construct
  misc_feature 1..22: note = strands RNA
  source 1..22: mol_type = other RNA
caagcgtcatcttttacgtggg

SEQ ID NO: 21 - RNA, 22 nt, synthetic construct
  misc_feature 1..22: note = strands RNA
  source 1..22: mol_type = other RNA
agcgagtactgtgagagcaagt

SEQ ID NO: 22 - RNA, 22 nt, synthetic construct
  misc_feature 1..22: note = strands RNA
  source 1..22: mol_type = other RNA
cagcgagtactgtgagagcaag

SEQ ID NO: 23 - RNA, 22 nt, synthetic construct
  misc_feature 1..22: note = strands RNA
  source 1..22: mol_type = other RNA
tcagcgagtactgtgagagcaa

SEQ ID NO: 24 - RNA, 22 nt, synthetic construct
  misc_feature 1..22: note = strands RNA
  source 1..22: mol_type = other RNA
caccctcagcgagtactgtgag

SEQ ID NO: 25 - RNA, 22 nt, synthetic construct
  misc_feature 1..22: note = strands RNA
  source 1..22: mol_type = other RNA
tcaccctcagcgagtactgtga

SEQ ID NO: 26 - RNA, 22 nt, synthetic construct
  misc_feature 1..22: note = strands RNA
  source 1..22: mol_type = other RNA
ttcaccctcagcgagtactgtg

SEQ ID NO: 27 - RNA, 22 nt, synthetic construct
  misc_feature 1..22: note = strands RNA
  source 1..22: mol_type = other RNA
gttcaccctcagcgagtactgt

SEQ ID NO: 28 - RNA, 22 nt, synthetic construct
  misc_feature 1..22: note = strands RNA
  source 1..22: mol_type = other RNA
tgttcaccctcagcgagtactg

SEQ ID NO: 29 - RNA, 22 nt, synthetic construct
  misc_feature 1..22: note = strands RNA
  source 1..22: mol_type = other RNA
cttgttcaccctcagcgagtac

SEQ ID NO: 30 - RNA, 22 nt, synthetic construct
  misc_feature 1..22: note = strands RNA
  source 1..22: mol_type = other RNA
tcttgttcaccctcagcgagta

SEQ ID NO: 31 - RNA, 22 nt, synthetic construct
  misc_feature 1..22: note = strands RNA
  source 1..22: mol_type = other RNA
ttcttgttcaccctcagcgagt

SEQ ID NO: 32 - RNA, 22 nt, synthetic construct
  misc_feature 1..22: note = strands RNA
  source 1..22: mol_type = other RNA
aggtcttttcttgttcaccctc

SEQ ID NO: 33 - RNA, 22 nt, synthetic construct
  misc_feature 1..22: note = strands RNA
  source 1..22: mol_type = other RNA
caggtcttttcttgttcaccct

SEQ ID NO: 34 - RNA, 22 nt, synthetic construct
  misc_feature 1..22: note = strands RNA
  source 1..22: mol_type = other RNA
tcaggtcttttcttgttcaccc

SEQ ID NO: 35 - RNA, 22 nt, synthetic construct
  misc_feature 1..22: note = strands RNA
  source 1..22: mol_type = other RNA
atcaggtcttttcttgttcacc

SEQ ID NO: 36 - RNA, 22 nt, synthetic construct
  misc_feature 1..22: note = strands RNA
  source 1..22: mol_type = other RNA
tatcaggtcttttcttgttcac

SEQ ID NO: 37 - RNA, 22 nt, synthetic construct
  misc_feature 1..22: note = strands RNA
  source 1..22: mol_type = other RNA
ttatcaggtcttttcttgttca

SEQ ID NO: 38 - RNA, 22 nt, synthetic construct
  misc_feature 1..22: note = strands RNA
  source 1..22: mol_type = other RNA
ctttatcaggtcttttcttgtt

SEQ ID NO: 39 - RNA, 22 nt, synthetic construct
  misc_feature 1..22: note = strands RNA
  source 1..22: mol_type = other RNA
ggttaatctttatcaggtcttt

SEQ ID NO: 40 - RNA, 22 nt, synthetic construct
  misc_feature 1..22: note = strands RNA
  source 1..22: mol_type = other RNA
tggttaatctttatcaggtctt

SEQ ID NO: 41 - RNA, 22 nt, synthetic construct
  misc_feature 1..22: note = strands RNA
  source 1..22: mol_type = other RNA
ctggttaatctttatcaggtct

SEQ ID NO: 42 - RNA, 22 nt, synthetic construct
  misc_feature 1..22: note = strands RNA
  source 1..22: mol_type = other RNA
tctggttaatctttatcaggtc

SEQ ID NO: 43 - RNA, 22 nt, synthetic construct
  misc_feature 1..22: note = strands RNA
  source 1..22: mol_type = other RNA
ttctggttaatctttatcaggt

SEQ ID NO: 44 - RNA, 22 nt, synthetic construct
  misc_feature 1..22: note = strands RNA
  source 1..22: mol_type = other RNA
tccttgttttcttctggttaat

SEQ ID NO: 45 - RNA, 22 nt, synthetic construct
  misc_feature 1..22: note = strands RNA
  source 1..22: mol_type = other RNA
gcggttgtttccctccttgttt

SEQ ID NO: 46 - RNA, 22 nt, synthetic construct
  misc_feature 1..22: note = strands RNA
  source 1..22: mol_type = other RNA
tgcggttgtttccctccttgtt

SEQ ID NO: 47 - RNA, 22 nt, synthetic construct
  misc_feature 1..22: note = strands RNA
  source 1..22: mol_type = other RNA
ctgcggttgtttccctccttgt

SEQ ID NO: 48 - RNA, 22 nt, synthetic construct
  misc_feature 1..22: note = strands RNA
  source 1..22: mol_type = other RNA
gctgcggttgtttccctccttg

SEQ ID NO: 49 - RNA, 22 nt, synthetic construct
  misc_feature 1..22: note = strands RNA
  source 1..22: mol_type = other RNA
tgctacaggctgcggttgtttc

SEQ ID NO: 50 - RNA, 22 nt, synthetic construct
  misc_feature 1..22: note = strands RNA
  source 1..22: mol_type = other RNA
ttgctacaggctgcggttgttt

SEQ ID NO: 51 - RNA, 22 nt, synthetic construct
  misc_feature 1..22: note = strands RNA
  source 1..22: mol_type = other RNA
cttgctacaggctgcggttgtt

SEQ ID NO: 52 - RNA, 22 nt, synthetic construct
  misc_feature 1..22: note = strands RNA
  source 1..22: mol_type = other RNA
gagcttgctacaggctgcggtt

SEQ ID NO: 53 - RNA, 22 nt, synthetic construct
  misc_feature 1..22: note = strands RNA
  source 1..22: mol_type = other RNA
agagcttgctacaggctgcggt

SEQ ID NO: 54 - RNA, 22 nt, synthetic construct
  misc_feature 1..22: note = strands RNA
  source 1..22: mol_type = other RNA
agttccagagcttgctacaggc

SEQ ID NO: 55 - RNA, 22 nt, synthetic construct
  misc_feature 1..22: note = strands RNA
  source 1..22: mol_type = other RNA
gagttccagagcttgctacagg

SEQ ID NO: 56 - RNA, 22 nt, synthetic construct
  misc_feature 1..22: note = strands RNA
  source 1..22: mol_type = other RNA
tgagttccagagcttgctacag

SEQ ID NO: 57 - RNA, 22 nt, synthetic construct
  misc_feature 1..22: note = strands RNA
  source 1..22: mol_type = other RNA
ctgagttccagagcttgctaca

SEQ ID NO: 58 - RNA, 22 nt, synthetic construct
  misc_feature 1..22: note = strands RNA
  source 1..22: mol_type = other RNA
cgactcctgagttccagagctt

SEQ ID NO: 59 - RNA, 22 nt, synthetic construct
  misc_feature 1..22: note = strands RNA
  source 1..22: mol_type = other RNA
cgcgactcctgagttccagagc

SEQ ID NO: 60 - RNA, 22 nt, synthetic construct
  misc_feature 1..22: note = strands RNA
  source 1..22: mol_type = other RNA
cgcgcgactcctgagttccaga

SEQ ID NO: 61 - RNA, 22 nt, synthetic construct
  misc_feature 1..22: note = strands RNA
  source 1..22: mol_type = other RNA
cctagcgcgcgactcctgagtt

SEQ ID NO: 62 - RNA, 22 nt, synthetic construct
  misc_feature 1..22: note = strands RNA
  source 1..22: mol_type = other RNA
acgtaaaagatgacgcttggtg

SEQ ID NO: 63 - RNA, 22 nt, synthetic construct
  misc_feature 1..22: note = strands RNA
  source 1..22: mol_type = other RNA
tccgcccacgtaaaagatgacg

SEQ ID NO: 64 - RNA, 22 nt, synthetic construct
  misc_feature 1..22: note = strands RNA
  source 1..22: mol_type = other RNA
cggtgtcccgctaggaaagaga

SEQ ID NO: 65 - RNA, 22 nt, synthetic construct
  misc_feature 1..22: note = strands RNA
  source 1..22: mol_type = other RNA
tgtgggtttaggaggtgtgtgt

SEQ ID NO: 66 - RNA, 22 nt, synthetic construct
  misc_feature 1..22: note = strands RNA
  source 1..22: mol_type = other RNA
agctccgagatgacacagactt

SEQ ID NO: 67 - RNA, 22 nt, synthetic construct
  misc_feature 1..22: note = strands RNA
  source 1..22: mol_type = other RNA
tgcttcacagctccgagatgac

SEQ ID NO: 68 - RNA, 22 nt, synthetic construct
  misc_feature 1..22: note = strands RNA
  source 1..22: mol_type = other RNA
acagacttgcttaaaggaagtg

SEQ ID NO: 69 - RNA, 18 nt, synthetic construct
  misc_feature 1..18: note = strands RNA
  source 1..18: mol_type = other RNA
cctacggtgtcccgctac

SEQ ID NO: 70 - RNA, 18 nt, synthetic construct
  misc_feature 1..18: note = strands RNA
  source 1..18: mol_type = other RNA
tacggtgtcccgctaggc

SEQ ID NO: 71 - RNA, 18 nt, synthetic construct
  misc_feature 1..18: note = strands RNA
  source 1..18: mol_type = other RNA
taggaaagagaggtgcgc

SEQ ID NO: 72 - RNA, 18 nt, synthetic construct
  misc_feature 1..18: note = strands RNA
  source 1..18: mol_type = other RNA
ggaaagagaggtgcgtcc

SEQ ID NO: 73 - RNA, 18 nt, synthetic construct
  misc_feature 1..18: note = strands RNA
  source 1..18: mol_type = other RNA
gaaagagaggtgcgtcac

SEQ ID NO: 74 - RNA, 18 nt, synthetic construct
  misc_feature 1..18: note = strands RNA
  source 1..18: mol_type = other RNA
aaagagaggtgcgtcaac

SEQ ID NO: 75 - RNA, 18 nt, synthetic construct
  misc_feature 1..18: note = strands RNA
  source 1..18: mol_type = other RNA
aggtgcgtcaaacagcgc

SEQ ID NO: 76 - RNA, 18 nt, synthetic construct
  misc_feature 1..18: note = strands RNA
  source 1..18: mol_type = other RNA
gtgcgtcaaacagcgacc

SEQ ID NO: 77 - RNA, 18 nt, synthetic construct
  misc_feature 1..18: note = strands RNA
  source 1..18: mol_type = other RNA
tgcgtcaaacagcgacac

SEQ ID NO: 78 - skipped

SEQ ID NO: 79 - RNA, 18 nt, synthetic construct
  misc_feature 1..18: note = strands RNA
  source 1..18: mol_type = other RNA
cgtcaaacagcgacaagc

SEQ ID NO: 80 - RNA, 18 nt, synthetic construct
  misc_feature 1..18: note = strands RNA
  source 1..18: mol_type = other RNA
acaagttccgcccacgtc

SEQ ID NO: 81 - RNA, 18 nt, synthetic construct
  misc_feature 1..18: note = strands RNA
  source 1..18: mol_type = other RNA
caagttccgcccacgtac

SEQ ID NO: 82 - RNA, 18 nt, synthetic construct
  misc_feature 1..18: note = strands RNA
  source 1..18: mol_type = other RNA
aagttccgcccacgtaac

SEQ ID NO: 83 - RNA, 18 nt, synthetic construct
  misc_feature 1..18: note = strands RNA
  source 1..18: mol_type = other RNA
agttccgcccacgtaaac

SEQ ID NO: 84 - RNA, 18 nt, synthetic construct
  misc_feature 1..18: note = strands RNA
  source 1..18: mol_type = other RNA
cacgtaaaagatgacgcc

SEQ ID NO: 85 - RNA, 18 nt, synthetic construct
  misc_feature 1..18: note = strands RNA
  source 1..18: mol_type = other RNA
acgtaaaagatgacgctc

SEQ ID NO: 86 - RNA, 18 nt, synthetic construct
  misc_feature 1..18: note = strands RNA
  source 1..18: mol_type = other RNA
cgtaaaagatgacgcttc

SEQ ID NO: 87 - RNA, 18 nt, synthetic construct
  misc_feature 1..18: note = strands RNA
  source 1..18: mol_type = other RNA
gctctcacagtactcgcc

SEQ ID NO: 88 - RNA, 18 nt, synthetic construct
  misc_feature 1..18: note = strands RNA
  source 1..18: mol_type = other RNA
ctctcacagtactcgctc

SEQ ID NO: 89 - RNA, 18 nt, synthetic construct
  misc_feature 1..18: note = strands RNA
  source 1..18: mol_type = other RNA
tctcacagtactcgctgc

SEQ ID NO: 90 - RNA, 18 nt, synthetic construct
  misc_feature 1..18: note = strands RNA
  source 1..18: mol_type = other RNA
cagtactcgctgagggtc

SEQ ID NO: 91 - RNA, 18 nt, synthetic construct
  misc_feature 1..18: note = strands RNA
  source 1..18: mol_type = other RNA
agtactcgctgagggtgc

SEQ ID NO: 92 - RNA, 18 nt, synthetic construct
  misc_feature 1..18: note = strands RNA
  source 1..18: mol_type = other RNA
gtactcgctgagggtgac

SEQ ID NO: 93 - RNA, 18 nt, synthetic construct
  misc_feature 1..18: note = strands RNA
  source 1..18: mol_type = other RNA
tactcgctgagggtgaaa

SEQ ID NO: 94 - RNA, 18 nt, synthetic construct
  misc_feature 1..18: note = strands RNA
  source 1..18: mol_type = other RNA
actcgctgagggtgaacc

SEQ ID NO: 95 - RNA, 18 nt, synthetic construct
  misc_feature 1..18: note = strands RNA
  source 1..18: mol_type = other RNA
tcgctgagggtgaacaac

SEQ ID NO: 96 - RNA, 18 nt, synthetic construct
  misc_feature 1..18: note = strands RNA
  source 1..18: mol_type = other RNA
cgctgagggtgaacaagc

SEQ ID NO: 97 - RNA, 18 nt, synthetic construct
  misc_feature 1..18: note = strands RNA
  source 1..18: mol_type = other RNA
gctgagggtgaacaagac

SEQ ID NO: 98 - RNA, 18 nt, synthetic construct
  misc_feature 1..18: note = strands RNA
  source 1..18: mol_type = other RNA
gtgaacaagaaaagaccc

SEQ ID NO: 99 - RNA, 18 nt, synthetic construct
  misc_feature 1..18: note = strands RNA
  source 1..18: mol_type = other RNA
tgaacaagaaaagacctc

SEQ ID NO: 100 - RNA, 18 nt, synthetic construct
  misc_feature 1..18: note = strands RNA
  source 1..18: mol_type = other RNA
gaacaagaaaagacctgc

SEQ ID NO: 101 - RNA, 18 nt, synthetic construct
  misc_feature 1..18: note = strands RNA
  source 1..18: mol_type = other RNA
aacaagaaaagacctgac

SEQ ID NO: 102 - RNA, 18 nt, synthetic construct
  misc_feature 1..18: note = strands RNA
  source 1..18: mol_type = other RNA
acaagaaaagacctgatc

SEQ ID NO: 103 - RNA, 18 nt, synthetic construct
  misc_feature 1..18: note = strands RNA
  source 1..18: mol_type = other RNA
caagaaaagacctgatac

SEQ ID NO: 104 - RNA, 18 nt, synthetic construct
  misc_feature 1..18: note = strands RNA
  source 1..18: mol_type = other RNA
agaaaagacctgataaac

SEQ ID NO: 105 - RNA, 18 nt, synthetic construct
  misc_feature 1..18: note = strands RNA
  source 1..18: mol_type = other RNA
acctgataaagattaaca

SEQ ID NO: 106 - RNA, 18 nt, synthetic construct
  misc_feature 1..18: note = strands RNA
  source 1..18: mol_type = other RNA
cctgataaagattaaccc

SEQ ID NO: 107 - RNA, 18 nt, synthetic construct
  misc_feature 1..18: note = strands RNA
  source 1..18: mol_type = other RNA
ctgataaagattaaccac

SEQ ID NO: 108 - RNA, 18 nt, synthetic construct
  misc_feature 1..18: note = strands RNA
  source 1..18: mol_type = other RNA
tgataaagattaaccagc

SEQ ID NO: 109 - RNA, 18 nt, synthetic construct
  misc_feature 1..18: note = strands RNA
  source 1..18: mol_type = other RNA
gataaagattaaccagac

SEQ ID NO: 110 - RNA, 18 nt, synthetic construct
  misc_feature 1..18: note = strands RNA
  source 1..18: mol_type = other RNA
accagaagaaaacaaggc

SEQ ID NO: 111 - RNA, 18 nt, synthetic construct
  misc_feature 1..18: note = strands RNA
  source 1..18: mol_type = other RNA
aaggagggaaacaaccga

SEQ ID NO: 112 - RNA, 18 nt, synthetic construct
  misc_feature 1..18: note = strands RNA
  source 1..18: mol_type = other RNA
aggagggaaacaaccgcc

SEQ ID NO: 113 - RNA, 18 nt, synthetic construct
  misc_feature 1..18: note = strands RNA
  source 1..18: mol_type = other RNA
ggagggaaacaaccgcac

SEQ ID NO: 114 - RNA, 18 nt, synthetic construct
  misc_feature 1..18: note = strands RNA
  source 1..18: mol_type = other RNA
gagggaaacaaccgcaga

SEQ ID NO: 115 - RNA, 18 nt, synthetic construct
  misc_feature 1..18: note = strands RNA
  source 1..18: mol_type = other RNA
caaccgcagcctgtagcc

SEQ ID NO: 116 - RNA, 18 nt, synthetic construct
  misc_feature 1..18: note = strands RNA
  source 1..18: mol_type = other RNA
aaccgcagcctgtagcac

SEQ ID NO: 117 - RNA, 18 nt, synthetic construct
  misc_feature 1..18: note = strands RNA
  source 1..18: mol_type = other RNA
accgcagcctgtagcaac

SEQ ID NO: 118 - RNA, 18 nt, synthetic construct
  misc_feature 1..18: note = strands RNA
  source 1..18: mol_type = other RNA
gcagcctgtagcaagcta

SEQ ID NO: 119 - RNA, 18 nt, synthetic construct
  misc_feature 1..18: note = strands RNA
  source 1..18: mol_type = other RNA
cagcctgtagcaagctcc

SEQ ID NO: 120 - RNA, 18 nt, synthetic construct
  misc_feature 1..18: note = strands RNA
  source 1..18: mol_type = other RNA
gtagcaagctctggaacc

SEQ ID NO: 121 - RNA, 18 nt, synthetic construct
  misc_feature 1..18: note = strands RNA
  source 1..18: mol_type = other RNA
tagcaagctctggaacta

SEQ ID NO: 122 - RNA, 18 nt, synthetic construct
  misc_feature 1..18: note = strands RNA
  source 1..18: mol_type = other RNA
agcaagctctggaactcc

SEQ ID NO: 123 - RNA, 18 nt, synthetic construct
  misc_feature 1..18: note = strands RNA
  source 1..18: mol_type = other RNA
gcaagctctggaactcac

SEQ ID NO: 124 - RNA, 18 nt, synthetic construct
  misc_feature 1..18: note = strands RNA
  source 1..18: mol_type = other RNA
tctggaactcaggagtcc

SEQ ID NO: 125 - RNA, 18 nt, synthetic construct
  misc_feature 1..18: note = strands RNA
  source 1..18: mol_type = other RNA
tggaactcaggagtcgcc

SEQ ID NO: 126 - RNA, 18 nt, synthetic construct
  misc_feature 1..18: note = strands RNA
  source 1..18: mol_type = other RNA
gaactcaggagtcgcgcc

SEQ ID NO: 127 - RNA, 18 nt, synthetic construct
  misc_feature 1..18: note = strands RNA
  source 1..18: mol_type = other RNA
caggagtcgcgcgctagc

SEQ ID NO: 128 - RNA, 18 nt, synthetic construct
  misc_feature 1..18: note = strands RNA
  source 1..18: mol_type = other RNA
aagcgtcatcttttacgc

SEQ ID NO: 129 - RNA, 18 nt, synthetic construct
  misc_feature 1..18: note = strands RNA
  source 1..18: mol_type = other RNA
atcttttacgtgggcggc

SEQ ID NO: 130 - RNA, 18 nt, synthetic construct
  misc_feature 1..18: note = strands RNA
  source 1..18: mol_type = other RNA
tttcctagcgggacaccc

SEQ ID NO: 131 - RNA, 18 nt, synthetic construct
  misc_feature 1..18: note = strands RNA
  source 1..18: mol_type = other RNA
acacctcctaaacccacc

SEQ ID NO: 132 - RNA, 18 nt, synthetic construct
  misc_feature 1..18: note = strands RNA
  source 1..18: mol_type = other RNA
ctgtgtcatctcggagcc

SEQ ID NO: 133 - RNA, 18 nt, synthetic construct
  misc_feature 1..18: note = strands RNA
  source 1..18: mol_type = other RNA
tctcggagctgtgaagcc

SEQ ID NO: 134 - RNA, 18 nt, synthetic construct
  misc_feature 1..18: note = strands RNA
  source 1..18: mol_type = other RNA
tcctttaagcaagtctgc

SEQ ID NO: 135 - RNA, 22 nt, synthetic construct
  misc_feature 1..22: note = First strand RNA sequence targeting sense C9orf72 exon 1acandidate 4
  source 1..22: mol_type = other RNA
gacgcacctctctttcctagcg

SEQ ID NO: 136 - RNA, 22 nt, synthetic construct
  misc_feature 1..22: note = First strand RNA sequence targeting sense C9orf72 exon 1acandidate 8
  source 1..22: mol_type = other RNA
gtttgacgcacctctctttcct

SEQ ID NO: 137 - RNA, 22 nt, synthetic construct
  misc_feature 1..22: note = First strand RNA sequence targeting sense C9orf72 exon 1acandidate 10
  source 1..22: mol_type = other RNA
gtcgctgtttgacgcacctctc

SEQ ID NO: 138 - RNA, 22 nt, synthetic construct
  misc_feature 1..22: note = First strand RNA sequence targeting sense C9orf72 exon 1acandidate 13
  source 1..22: mol_type = other RNA
cttgtcgctgtttgacgcacct

SEQ ID NO: 139 - RNA, 22 nt, synthetic construct
  misc_feature 1..22: note = First strand RNA sequence targeting sense C9orf72 exon 1acandidate 15
  source 1..22: mol_type = other RNA
gcggaacttgtcgctgtttgac

SEQ ID NO: 140 - RNA, 22 nt, synthetic construct
  misc_feature 1..22: note = First strand RNA sequence targeting sense C9orf72 exon 1acandidate 16
  source 1..22: mol_type = other RNA
ggcggaacttgtcgctgtttga

SEQ ID NO: 141 - RNA, 22 nt, synthetic construct
  misc_feature 1..22: note = First strand RNA sequence targeting sense C9orf72 exon 1acandidate 17
  source 1..22: mol_type = other RNA
tgggcggaacttgtcgctgttt

SEQ ID NO: 142 - RNA, 22 nt, synthetic construct
  misc_feature 1..22: note = First strand RNA sequence targeting sense C9orf72 exon 1acandidate 22
  source 1..22: mol_type = other RNA
cgtcatcttttacgtgggcgga

SEQ ID NO: 143 - RNA, 22 nt, synthetic construct
  misc_feature 1..22: note = First strand RNA sequence targeting sense C9orf72 exon 1acandidate 26
  source 1..22: mol_type = other RNA
gcgtcatcttttacgtgggcgg

SEQ ID NO: 144 - RNA, 22 nt, synthetic construct
  misc_feature 1..22: note = First strand RNA sequence targeting sense C9orf72 exon 1acandidate 27
  source 1..22: mol_type = other RNA
gtactgtgagagcaagtagtgg

SEQ ID NO: 145 - RNA, 22 nt, synthetic construct
  misc_feature 1..22: note = First strand RNA sequence targeting sense C9orf72 exon 1acandidate 28
  source 1..22: mol_type = other RNA
agtactgtgagagcaagtagtg

SEQ ID NO: 146 - RNA, 22 nt, synthetic construct
  misc_feature 1..22: note = First strand RNA sequence targeting sense C9orf72 exon 1acandidate 29
  source 1..22: mol_type = other RNA
gagtactgtgagagcaagtagt

SEQ ID NO: 147 - RNA, 22 nt, synthetic construct
  misc_feature 1..22: note = First strand RNA sequence targeting sense C9orf72 exon 1acandidate 30
  source 1..22: mol_type = other RNA
cgagtactgtgagagcaagtag

SEQ ID NO: 148 - RNA, 22 nt, synthetic construct
  misc_feature 1..22: note = First strand RNA sequence targeting sense C9orf72 exon 1acandidate 31
  source 1..22: mol_type = other RNA
gcgagtactgtgagagcaagta

SEQ ID NO: 149 - RNA, 22 nt, synthetic construct
  misc_feature 1..22: note = First strand RNA sequence targeting sense C9orf72 exon 1acandidate 35
  source 1..22: mol_type = other RNA
ctcagcgagtactgtgagagca

SEQ ID NO: 150 - RNA, 22 nt, synthetic construct
  misc_feature 1..22: note = First strand RNA sequence targeting sense C9orf72 exon 1acandidate 50
  source 1..22: mol_type = other RNA
tttatcaggtcttttcttgttc

SEQ ID NO: 151 - RNA, 22 nt, synthetic construct
  misc_feature 1..22: note = First strand RNA sequence targeting sense C9orf72 exon 1acandidate 58
  source 1..22: mol_type = other RNA
ggttgtttccctccttgttttc

SEQ ID NO: 152 - RNA, 22 nt, synthetic construct
  misc_feature 1..22: note = First strand RNA sequence targeting sense C9orf72 exon 1acandidate 59
  source 1..22: mol_type = other RNA
cggttgtttccctccttgtttt

SEQ ID NO: 153 - RNA, 22 nt, synthetic construct
  misc_feature 1..22: note = First strand RNA sequence targeting sense C9orf72 exon 1acandidate 64
  source 1..22: mol_type = other RNA
ggctgcggttgtttccctcctt

SEQ ID NO: 154 - RNA, 22 nt, synthetic construct
  misc_feature 1..22: note = First strand RNA sequence targeting sense C9orf72 exon 1acandidate 65
  source 1..22: mol_type = other RNA
gctacaggctgcggttgtttcc

SEQ ID NO: 155 - RNA, 22 nt, synthetic construct
  misc_feature 1..22: note = First strand RNA sequence targeting sense C9orf72 exon 1acandidate 76
  source 1..22: mol_type = other RNA
gcgactcctgagttccagagct

SEQ ID NO: 156 - RNA, 22 nt, synthetic construct
  misc_feature 1..22: note = Scramble candidate number 1
  source 1..22: mol_type = other RNA
gcgacttgtaacgtcagaggcg

SEQ ID NO: 157 - RNA, 22 nt, synthetic construct
  misc_feature 1..22: note = Scramble candidate number 28
  source 1..22: mol_type = other RNA
attggctcgtctctcatctcgc

SEQ ID NO: 158 - RNA, 22 nt, synthetic construct
  misc_feature 1..22: note = Scramble candidate number 55
  source 1..22: mol_type = other RNA
acgatgtccggtgtgctcattg

SEQ ID NO: 159 - RNA, 22 nt, synthetic construct
  misc_feature 1..22: note = Scramble candidate number 92
  source 1..22: mol_type = other RNA
aggtgcgcgattagtggtagaa

SEQ ID NO: 160 - RNA, 22 nt, synthetic construct
  misc_feature 1..22: note = Scramble candidate number 131
  source 1..22: mol_type = other RNA
acttgtgtctattatcttctat

SEQ ID NO: 161 - RNA, 22 nt, synthetic construct
  misc_feature 1..22: note = Scramble candidate number 195
  source 1..22: mol_type = other RNA
acgccggtactgcgctacactg

SEQ ID NO: 162 - RNA, 22 nt, synthetic construct
  misc_feature 1..22: note = Scramble candidate number ALS001
  source 1..22: mol_type = other RNA
gtagttctattagcgcttacta

SEQ ID NO: 163 - DNA, 1071 bp, Homo sapiens
  source 1..1071: mol_type = other DNA
acgtaacctacggtgtcccgctaggaaagagaggtgcgtcaaacagcgacaagttccgcccacgtaaaagatgacgcttggtgtgtcagccgtccctgctgcccggttgcttctcttttgggggcggggtctagcaagagcaggtgtgggtttaggaggtgtgtgtttttgtttttcccaccctctctccccactacttgctctcacagtactcgctgagggtgaacaagaaaagacctgataaagattaaccagaagaaaacaaggagggaaacaaccgcagcctgtagcaagctctggaactcaggagtcgcgcgctaggggccggggccggggccggggcgtggtcggggcgggcccgggggcgggcccggggcggggctgcggttgcggtgcctgcgcccgcggcggcggaggcgcaggcggtggcgagtgggtgagtgaggaggcggcatcctggcgggtggctgtttggggttcggctgccgggaagaggcgcgggtagaagcgggggctctcctcagagctcgacgcatttttactttccctctcatttctctgaccgaagctgggtgtcgggctttcgcctctagcgactggtggaattgcctgcatccgggccccgggcttcccggcggcggcggcggcggcggcggcgcagggacaagggatggggatctggcctcttccttgctttcccgccctcagtacccgagctgtctccttcccggggacccgctgggagcgctgccgctgcgggctcgagaaaagggagcctcgggtactgagaggcctcgcctgggggaaggccggagggtgggcggcgcgcggcttctgcggaccaagtcggggttcgctaggaacccgagacggtccctgccggcgaggagatcatgcgggatgagatgggggtgtggagacgcctgcacaatttcagcccaagcttctagagagtggtgatgacttgcatatgagggcagcaatgcaagtcggtgtgctccccattctgtgggacatgacctggttgcttcacagctccgagatgacacagacttgcttaaaggaagtgac

SEQ ID NO: 164 - RNA, 22 nt, synthetic construct
  misc_feature 1..22: note = First strand RNA sequence targeting antisense C9orf72 spanningexon 1a and intron 1 candidate 16
  source 1..22: mol_type = other RNA
aggtgtgggtttaggaggtgtg

SEQ ID NO: 165 - RNA, 22 nt, synthetic construct
  misc_feature 1..22: note = First strand RNA sequence targeting antisense C9orf72 intron 1candidate 1
  source 1..22: mol_type = other RNA
agacttgcttaaaggaagtgac

SEQ ID NO: 166 - RNA, 22 nt, synthetic construct
  misc_feature 1..22: note = First strand RNA sequence targeting antisense C9orf72 intron 1candidate 2
  source 1..22: mol_type = other RNA
tccgagatgacacagacttgct

SEQ ID NO: 167 - RNA, 22 nt, synthetic construct
  misc_feature 1..22: note = First strand RNA sequence targeting antisense C9orf72 intron 1candidate 5
  source 1..22: mol_type = other RNA
acttgcatatgagggcagcaat

SEQ ID NO: 168 - RNA, 22 nt, synthetic construct
  misc_feature 1..22: note = First strand RNA sequence targeting antisense C9orf72 intron 1candidate 6
  source 1..22: mol_type = other RNA
tctagcgactggtggaattgcc

SEQ ID NO: 169 - RNA, 22 nt, synthetic construct
  misc_feature 1..22: note = First strand RNA sequence targeting antisense C9orf72 intron 1candidate 7
  source 1..22: mol_type = other RNA
tttcgcctctagcgactggtgg

SEQ ID NO: 170 - RNA, 22 nt, synthetic construct
  misc_feature 1..22: note = First strand RNA sequence targeting antisense C9orf72 intron 1candidate 8
  source 1..22: mol_type = other RNA
agggtgaacaagaaaagacctg

SEQ ID NO: 171 - RNA, 22 nt, synthetic construct
  misc_feature 1..22: note = First strand RNA sequence targeting antisense C9orf72 intron 1candidate 9
  source 1..22: mol_type = other RNA
tactcgctgagggtgaacaaga

SEQ ID NO: 172 - RNA, 22 nt, synthetic construct
  misc_feature 1..22: note = First strand RNA sequence targeting antisense C9orf72 intron 1candidate 10
  source 1..22: mol_type = other RNA
agtactcgctgagggtgaacaa

SEQ ID NO: 173 - RNA, 22 nt, synthetic construct
  misc_feature 1..22: note = First strand RNA sequence targeting antisense C9orf72 intron 1candidate 11
  source 1..22: mol_type = other RNA
acagtactcgctgagggtgaac

SEQ ID NO: 174 - RNA, 22 nt, synthetic construct
  misc_feature 1..22: note = First strand RNA sequence targeting antisense C9orf72 intron 1candidate 12
  source 1..22: mol_type = other RNA
tgctctcacagtactcgctgag

SEQ ID NO: 175 - RNA, 22 nt, synthetic construct
  misc_feature 1..22: note = First strand RNA sequence targeting antisense C9orf72 intron 1candidate 13
  source 1..22: mol_type = other RNA
tacttgctctcacagtactcgc

SEQ ID NO: 176 - RNA, 22 nt, synthetic construct
  misc_feature 1..22: note = First strand RNA sequence targeting antisense C9orf72 intron 1candidate 14
  source 1..22: mol_type = other RNA
actacttgctctcacagtactc

SEQ ID NO: 177 - RNA, 22 nt, synthetic construct
  misc_feature 1..22: note = First strand RNA sequence targeting antisense C9orf72 intron 1candidate 21
  source 1..22: mol_type = other RNA
ctccgagatgacacagacttgc

SEQ ID NO: 178 - RNA, 22 nt, synthetic construct
  misc_feature 1..22: note = First strand RNA sequence targeting antisense C9orf72 intron 1candidate 22
  source 1..22: mol_type = other RNA
agcgactggtggaattgcctgc

SEQ ID NO: 179 - RNA, 22 nt, synthetic construct
  misc_feature 1..22: note = First strand RNA sequence targeting antisense C9orf72 intron 1candidate 23
  source 1..22: mol_type = other RNA
tagcgactggtggaattgcctg

SEQ ID NO: 180 - RNA, 22 nt, synthetic construct
  misc_feature 1..22: note = First strand RNA sequence targeting antisense C9orf72 intron 1candidate 24
  source 1..22: mol_type = other RNA
ctagcgactggtggaattgcct

SEQ ID NO: 181 - DNA, 86 bp, synthetic construct
  misc_feature 1..86: note = Mutated miR-144 helper hairpin
  source 1..86: mol_type = other DNA
tggggccctggctgggatttcatcatatactgtaagtttgcgatgagacactacagtatagatgatgtactagtccgggcaccccc

SEQ ID NO: 182 - DNA, 174 bp, synthetic construct
  misc_feature 1..174: note = V1S candidate 13 miR451 scaffolds
  source 1..174: mol_type = other DNA
cttgggaatggcaaggcaagcgtcatcttttacgtgggcgtaaaagatgacgcttctcttgctatacccagaaaacgtgccaggaagctgactgccagggcacttgggaatggcaaggacagacttgcttaaaggaagtgtcctttaagcaagtctgctcttgctatacccaga

SEQ ID NO: 183 - DNA, 174 bp, synthetic construct
  misc_feature 1..174: note = V1S candidate 14 miR451 scaffolds
  source 1..174: mol_type = other DNA
cttgggaatggcaaggtcaggtcttttcttgttcacccgaacaagaaaagacctgctcttgctatacccagaaaacgtgccaggaagctgactgccagggcacttgggaatggcaaggacagacttgcttaaaggaagtgtcctttaagcaagtctgctcttgctatacccaga

SEQ ID NO: 184 - DNA, 174 bp, synthetic construct
  misc_feature 1..174: note = V1S candidate 15 miR451 scaffolds
  source 1..174: mol_type = other DNA
cttgggaatggcaaggtctggttaatctttatcaggtctgataaagattaaccagctcttgctatacccagaaaacgtgccaggaagctgactgccagggcacttgggaatggcaaggacagacttgcttaaaggaagtgtcctttaagcaagtctgctcttgctatacccaga

SEQ ID NO: 185 - DNA, 174 bp, synthetic construct
  misc_feature 1..174: note = V1S candidate 16 miR451 scaffolds
  source 1..174: mol_type = other DNA
cttgggaatggcaaggacagacttgcttaaaggaagtgtcctttaagcaagtctgctcttgctatacccagaaaacgtgccaggaagctgactgccagggcacttgggaatggcaaggcaagcgtcatcttttacgtgggcgtaaaagatgacgcttctcttgctatacccaga

SEQ ID NO: 186 - DNA, 174 bp, synthetic construct
  misc_feature 1..174: note = V1S candidate 17 miR451 scaffolds
  source 1..174: mol_type = other DNA
cttgggaatggcaaggacagacttgcttaaaggaagtgtcctttaagcaagtctgctcttgctatacccagaaaacgtgccaggaagctgactgccagggcacttgggaatggcaaggtcaggtcttttcttgttcacccgaacaagaaaagacctgctcttgctatacccaga

SEQ ID NO: 187 - DNA, 174 bp, synthetic construct
  misc_feature 1..174: note = V1S candidate 18 miR451 scaffolds
  source 1..174: mol_type = other DNA
cttgggaatggcaaggacagacttgcttaaaggaagtgtcctttaagcaagtctgctcttgctatacccagaaaacgtgccaggaagctgactgccagggcacttgggaatggcaaggtctggttaatctttatcaggtctgataaagattaaccagctcttgctatacccaga

SEQ ID NO: 188 - DNA, 174 bp, synthetic construct
  misc_feature 1..174: note = V1S candidate 19 miR451 scaffolds
  source 1..174: mol_type = other DNA
cttgggaatggcaagggcgacttgtaacgtcagaggcgtctgacgttacaagtcgatcttgctatacccagaaaacgtgccaggaagctgactgccagggcacttgggaatggcaaggacagacttgcttaaaggaagtgtcctttaagcaagtctgctcttgctatacccaga

SEQ ID NO: 189 - DNA, 174 bp, synthetic construct
  misc_feature 1..174: note = V1S candidate 20 miR451 scaffolds
  source 1..174: mol_type = other DNA
cttgggaatggcaagggtagttctattagcgcttactaaagcgctaatagaactaatcttgctatacccagaaaacgtgccaggaagctgactgccagggcacttgggaatggcaagggcgacttgtaacgtcagaggcgtctgacgttacaagtcgatcttgctatacccaga

SEQ ID NO: 190 - DNA, 174 bp, synthetic construct
  misc_feature 1..174: note = V1S candidate 21 miR451 scaffolds
  source 1..174: mol_type = other DNA
cttgggaatggcaaggacagacttgcttaaaggaagtgtcctttaagcaagtctgctcttgctatacccagaaaacgtgccaggaagctgactgccagggcacttgggaatggcaagggcgacttgtaacgtcagaggcgtctgacgttacaagtcgatcttgctatacccaga

SEQ ID NO: 191 - DNA, 174 bp, synthetic construct
  misc_feature 1..174: note = V1S candidate 22 miR451 scaffolds
  source 1..174: mol_type = other DNA
cttgggaatggcaaggtcaggtcttttcttgttcacccgaacaagaaaagacctgctcttgctatacccagaaaacgtgccaggaagctgactgccagggcacttgggaatggcaagggcgacttgtaacgtcagaggcgtctgacgttacaagtcgatcttgctatacccaga

SEQ ID NO: 192 - DNA, 174 bp, synthetic construct
  misc_feature 1..174: note = V1S candidate 23 miR451 scaffolds
  source 1..174: mol_type = other DNA
cttgggaatggcaagggcgacttgtaacgtcagaggcgtctgacgttacaagtcgatcttgctatacccagaaaacgtgccaggaagctgactgccagggcacttgggaatggcaaggtcaggtcttttcttgttcacccgaacaagaaaagacctgctcttgctatacccaga

SEQ ID NO: 193 - DNA, 72 bp, synthetic construct
  misc_feature 1..72: note = V2S candidate 42 miR451 scaffold
  source 1..72: mol_type = other DNA
cttgggaatggcaaggacagacttgcttaaaggaagtgtcctttaagcaagtctgctcttgctatacccaga

SEQ ID NO: 194 - DNA, 72 bp, synthetic construct; identical to SEQ ID NO: 193
  misc_feature 1..72: note = V2S candidate 43 miR451 scaffold
  source 1..72: mol_type = other DNA

SEQ ID NO: 195 - DNA, 72 bp, synthetic construct; identical to SEQ ID NO: 193
  misc_feature 1..72: note = V2S candidate 44 miR451 scaffold
  source 1..72: mol_type = other DNA

SEQ ID NO: 196 - DNA, 72 bp, synthetic construct; identical to SEQ ID NO: 193
  misc_feature 1..72: note = V2S candidate 45 miR451 scaffold
  source 1..72: mol_type = other DNA

SEQ ID NO: 197 - DNA, 72 bp, synthetic construct; identical to SEQ ID NO: 193
  misc_feature 1..72: note = V2S candidate 46 miR451 scaffold
  source 1..72: mol_type = other DNA

SEQ ID NO: 198 - DNA, 72 bp, synthetic construct; identical to SEQ ID NO: 193
  misc_feature 1..72: note = V2S candidate 47 miR451 scaffold
  source 1..72: mol_type = other DNA

SEQ ID NO: 199 - DNA, 72 bp, synthetic construct; identical to SEQ ID NO: 193
  misc_feature 1..72: note = V2S candidate 48 miR451 scaffold
  source 1..72: mol_type = other DNA

SEQ ID NO: 200 - DNA, 72 bp, synthetic construct; identical to SEQ ID NO: 193
  misc_feature 1..72: note = V2S candidate 49 miR451 scaffold
  source 1..72: mol_type = other DNA

SEQ ID NO: 201 - DNA, 72 bp, synthetic construct; identical to SEQ ID NO: 193
  misc_feature 1..72: note = V2S candidate 50 miR451 scaffold
  source 1..72: mol_type = other DNA

SEQ ID NO: 202 - DNA, 72 bp, synthetic construct
  misc_feature 1..72: note = V2S candidate 51 miR451 scaffold
  source 1..72: mol_type = other DNA
cttgggaatggcaagggtagttctattagcgcttactaaagcgctaatagaactaatcttgctatacccaga

SEQ ID NO: 203 - DNA, 72 bp, synthetic construct; identical to SEQ ID NO: 193
  misc_feature 1..72: note = V2S candidate 52 miR451 scaffold
  source 1..72: mol_type = other DNA

SEQ ID NO: 204 - DNA, 72 bp, synthetic construct; identical to SEQ ID NO: 193
  misc_feature 1..72: note = V2S candidate 53 miR451 scaffold
  source 1..72: mol_type = other DNA

SEQ ID NO: 205 - DNA, 1602 bp, synthetic construct
  misc_feature 1..1602: note = CMV immediate-early enhancer fused to chicken B-actin (CAG)promoter
  source 1..1602: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaagtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgcgccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 206 - DNA, 133 bp, synthetic construct
  misc_feature 1..133: note = simian virus 40 polyadenylation signal (SV40 polyA)
  source 1..133: mol_type = other DNA
tgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 207 - DNA, 777 bp, synthetic construct
  misc_feature 1..777: note = Alternative promoter
  source 1..777: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctga

SEQ ID NO: 208 - DNA, 320 bp, synthetic construct
  misc_feature 1..320: note = Insert dual luciferase reporter for sense miC9O screening
  source 1..320: mol_type = other DNA
acgtaacctacggtgtcccgctaggaaagagaggtgcgtcaaacagcgacaagttccgcccacgtaaaagatgacgcttggtgtgtcagccgtccctgctgcccggttgcttctcttttgggggcggggtctagcaagagcaggtgtgggtttaggaggtgtgtgtttttgtttttcccaccctctctccccactacttgctctcacagtactcgctgagggtgaacaagaaaagacctgataaagattaaccagaagaaaacaaggagggaaacaaccgcagcctgtagcaagctctggaactcaggagtcgcgcgcta

SEQ ID NO: 209 - DNA, 575 bp, synthetic construct
  misc_feature 1..575: note = Insert dual luciferase reporter for antisense miC9O screening
  source 1..575: mol_type = other DNA
gctagccaagtgatgcccaagtcacaatagtcacttcctttaagcaagtctgtgtcatctcggagctgtgaagcaaccaggtcatgtcccacagaatggggagcacaccgacttgcattgctgccctcatatgcaagtcatcaccactctctagaagcttgggctgaaattgtgcaggccgccgccgccgccgccgccgggaagcccggggcccggatgcaggcaattccaccagtcgctagaggcgaaagcccgacacccagcttcggtcagagaaatgagagggaaagttgtttccctccttgttttcttctggttaatctttatcaggtcttttcttgttcaccctcagcgagtactgtgagagcaagtagtggggagagagggtgggaaaaacaaaaacacacacctcctaaacccacacctgctcttgctagaccccgcccccaaaagagaagcaaccgggcagcagggacggctgacacaccaagcgtcatcttttacgtgggcggaacttgtcgctgtttgacgcacctctctttcctagcgggacaccgtaggttacgtgcggccgc

SEQ ID NO: 210 - DNA, 1182 bp, synthetic construct
  misc_feature 1..1182: note = Human elongation Factor-1 alpha (EF-1a) promoter
  source 1..1182: mol_type = other DNA
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 211 - DNA, 1360 bp, synthetic construct
  misc_feature 1..1360: note = Insert for artificial intronic C9orf72 construct
  source 1..1360: mol_type = other DNA
acgtaacctacggtgtcccgctaggaaagagaggtgcgtcaaacagcgacaagttccgcccacgtaaaagatgacgcttggtgtgtcagccgtccctgctgcccggttgcttctcttttgggggcggggtctagcaagagcaggtgtgggtttaggaggtgtgtgtttttgtttttcccaccctctctccccactacttgctctcacagtactcgctgagggtgaacaagaaaagacctgataaagattaaccagaagaaaacaaggagggaaacaaccgcagcctgtagcaagctctggaactcaggagtcgcgcgctaggggccggggccggggccggggcgtggtcggggcgggcccgggggcgggcccggggcggggctgcggttgcggtgcctgcgcccgcggcggcggaggcgcaggcggtggcgagtgggtgagtgaggaggcggcatcctggcgggtggctgtttggggttcggctgccgggaagaggcgcgggtagaagcgggggctctcctcagagctcgacgcatttttactttccctctcatttctctgaccgaagctgggtgtcgggctttcgcctctagcgactggtggaattgcctgcatccgggccccgggcttcccggcggcggcggcggcggcggcggcgcagggacaagggatggggatctggcctcttccttgctttcccgccctcagtacccgagctgtctccttcccggggacccgctgggagcgctgccgctgcgggctcgagaaaagggagcctcgggtactgagaggcctcgcctgggggaaggccggagggtgggcggcgcgcggcttctgcggaccaagtcggggttcgctaggaacccgagacggtccctgccggcgaggagatcatgcgggatgagatgggggtgtggagacgcctgcacaatttcagcccaagcttctagagagtggtgatgacttgcatatgagggcagcaatgcaagtcggtgtgctccccattctgtgggacatgacctggttgcttcacagctccgagatgacacagacttgcttaaaggaagtgactattgtgacttgggcatcacttgactgatggtaatcagttgtctaaagaagtgcacagattacatgtccgtgtgctcattgggtctatctggccgcgttgaacaccaccaggctttgtattcagaaacaggagggaggtcctgcactttcccaggaggggtggccctttcagatgcaatcgagattgttaggctctgggagagtagttgcctggttgtggcagttggtaaatttctattcaaacagttgccatgcaccagttgttcacatttcatatgtcattgtttag

SEQ ID NO: 212 - DNA, 22 bp, synthetic construct
  misc_feature 1..22: note = Forward primer to quantify intronic C9orf72 mRNA
  source 1..22: mol_type = other DNA
tgtgtttttgtttttcccaccc

SEQ ID NO: 213 - DNA, 22 bp, synthetic construct
  misc_feature 1..22: note = Reverse primer to quantify intronic C9orf72 mRNA
  source 1..22: mol_type = other DNA
cggttgtttccctccttgtttt

SEQ ID NO: 214 - DNA, 25 bp, synthetic construct
  misc_feature 1..25: note = Probe to quantify intronic C9orf72 mRNA
  source 1..25: mol_type = other DNA
ctgagggtgaacaagaaaagacctg

SEQ ID NO: 215 - DNA, 24 bp, synthetic construct
  misc_feature 1..24: note = Forward primer to quantify human ?-actin mRNA
  source 1..24: mol_type = other DNA
aggtgcacagtaggtctgaacaga

SEQ ID NO: 216 - DNA, 22 bp, synthetic construct
  misc_feature 1..22: note = Reverse primer to quantify human ?-actin mRNA
  source 1..22: mol_type = other DNA
tgcaaagaacacggctaagtgt

SEQ ID NO: 217 - DNA, 72 bp, synthetic construct
  misc_feature 1..72: note = pre-miRNA miC9026
  source 1..72: mol_type = other DNA
cttgggaatggcaaggcaagcgtcatcttttacgtgggcgtaaaagatgacgcttctcttgctatacccaga

SEQ ID NO: 218 - DNA, 72 bp, synthetic construct
  misc_feature 1..72: note = pri-miRNA miC9O26
  source 1..72: mol_type = other DNA
cttgggaatggcaaggtcaggtcttttcttgttcacccgaacaagaaaagacctgctcttgctatacccaga

SEQ ID NO: 219 - DNA, 72 bp, synthetic construct
  misc_feature 1..72: note = pri-miRNA miC9O55
  source 1..72: mol_type = other DNA
cttgggaatggcaaggtctggttaatctttatcaggtctgataaagattaaccagctcttgctatacccaga

SEQ ID NO: 220 - DNA, 2229 bp, synthetic construct
  misc_feature 1..2229: note = Expression cassette harboring miC9O26
  source 1..2229: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaagtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgcgccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctagcatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggcaagcgtcatcttttacgtgggcgtaaaagatgacgcttctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgctggatcctgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 221 - DNA, 495 bp, synthetic construct
  misc_feature 1..495: note = Expression cassette harboring V2S candidate 46
  source 1..495: mol_type = other DNA
gctagcatcggcgctatgcttcctgtgcccccagtggggccctggctgtctggttaatctttatcaggtctttgcgatgagagtccctgataaaattaaccaaacagtccgggcacccccagctctggagcctgacaaggaggacaggagagatgctgcaagcccaagaagctctctgctcagcctgtcacaacctactgactgccagggcacttgggaatggcaaggacagacttgcttaaaggaagtgtcctttaagcaagtctgctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgctggatcc

SEQ ID NO: 222 - DNA, 22 bp, synthetic construct
  misc_feature 1..22: note = Forward primer to quantify vector genome copies
  source 1..22: mol_type = other DNA
agtaacgccaatagggactttc

SEQ ID NO: 223 - DNA, 22 bp, synthetic construct
  misc_feature 1..22: note = Reverse primer to quantify vector genome copies
  source 1..22: mol_type = other DNA
ggcgtacttggcatatgataca

SEQ ID NO: 224 - DNA, 24 bp, synthetic construct
  misc_feature 1..24: note = Probe to quantify vector genome copies
  source 1..24: mol_type = other DNA
ttacggtaaactgcccacttggca

SEQ ID NO: 225 - DNA, 92 bp, synthetic construct
  misc_feature 1..92: note = 92 bp spacer region separating mir-144-mir-451 hairpins
  source 1..92: mol_type = other DNA
agctctggagcctgacaaggaggacaggagagatgctgcaagcccaagaagctctctgctcagcctgtcacaacctactgactgccagggca

SEQ ID NO: 226 - DNA, 92 bp, synthetic construct
  misc_feature 1..92: note = 92 bp spacer region separating mir-451-144 hairpins
  source 1..92: mol_type = other DNA
acgggaccgtcagtcatccaacactgtccgactcgtctctcgaagaacccgaacgtcgtagagaggacaggaggaacagtccgaggtctcga

SEQ ID NO: 227 - DNA, 30 bp, synthetic construct
  misc_feature 1..30: note = 30 bp spacer sequence separating mir-451-mir-451 hairpins
  source 1..30: mol_type = other DNA
aaacgtgccaggaagctgactgccagggca

SEQ ID NO: 228 - DNA, 86 bp, synthetic construct
  misc_feature 1..86: note = human pri-mir-144 sequence
  source 1..86: mol_type = other DNA
tggggccctggctgggatatcatcatatactgtaagtttgcgatgagacactacagtatagatgatgtactagtccgggcaccccc

SEQ ID NO: 229 - RNA, 72 nt, synthetic construct
  misc_feature 1..72: note = miR-451 scaffold
  misc_feature 17..55: note = n is a, c, g, t or u
  source 1..72: mol_type = other RNA
cttgggaatggcaaggnnnnnnnnnnnnnnnnnnnnnnnnnnnnnnnnnnnnnnnctcttgctatacccaga

SEQ ID NO: 230 - RNA, 86 nt, synthetic construct
  misc_feature 1..86: note = miR-144 scaffold comprising guide sequence
  misc_feature 15..36: note = n is a, c, g, t or u
  misc_feature 52..71: note = n is a, c, g, t or u
  source 1..86: mol_type = other RNA
tggggccctggctgnnnnnnnnnnnnnnnnnnnnnntttgcgatgagacacnnnnnnnnnnnnnnnnnnnnagtccgggcaccccc

SEQ ID NO: 231 - RNA, 16 nt, synthetic construct
  misc_feature 1..16: note = mir-451 hairpin stem 5
  source 1..16: mol_type = other RNA
cttgggaatggcaagg

SEQ ID NO: 232 - RNA, 17 nt, synthetic construct
  misc_feature 1..17: note = mir-451 hairpin stem 3
  source 1..17: mol_type = other RNA
mwcttgctatacccaga

SEQ ID NO: 233-234 - skipped (2 entries)

SEQ ID NO: 235 - RNA, 14 nt, synthetic construct
  misc_feature 1..14: note = mir-144 hairpin step 5
  source 1..14: mol_type = other RNA
tggggccctggctm

SEQ ID NO: 236 - RNA, 15 nt, synthetic construct
  misc_feature 1..15: note = mir-144 apical loop
  source 1..15: mol_type = other RNA
tttgcgatgagawmm

SEQ ID NO: 237 - RNA, 15 nt, synthetic construct
  misc_feature 1..15: note = mir-144 hairpin stem 3
  source 1..15: mol_type = other RNA
agtccgggcaccccc

SEQ ID NO: 238 - DNA, 28 bp, synthetic construct
  misc_feature 1..28: note = mir-144 scaffold
  source 1..28: mol_type = other DNA
atcggcgctatgcttcctgtgcccccag

SEQ ID NO: 239 - DNA, 22 bp, synthetic construct
  source 1..22: mol_type = other DNA
agggtgaacaagaaaagacctg

SEQ ID NO: 240 - DNA, 22 bp, synthetic construct
  source 1..22: mol_type = other DNA
agggtgaacaagaaaagacctg

SEQ ID NO: 241 - DNA, 17 bp, synthetic construct
  source 1..17: mol_type = other DNA
taaagattaaccagaag

SEQ ID NO: 242 - DNA, 20 bp, synthetic construct
  source 1..20: mol_type = other DNA
cggtggcgagtggatatctc

SEQ ID NO: 243 - DNA, 20 bp, synthetic construct
  source 1..20: mol_type = other DNA
tgggcaaagagtcgacatca

SEQ ID NO: 244 - DNA, 20 bp, synthetic construct
  source 1..20: mol_type = other DNA
taatgtgacagttggaatgc

SEQ ID NO: 245 - DNA, 26 bp, synthetic construct
  source 1..26: mol_type = other DNA
caggtccagtttttttttttttttvn

SEQ ID NO: 246 - DNA, 18 bp, synthetic construct
  source 1..18: mol_type = other DNA
aggcgacttgtaacgtca

SEQ ID NO: 247 - DNA, 26 bp, synthetic construct
  source 1..26: mol_type = other DNA
cagtttttttttttttttacgcctct

SEQ ID NO: 248 - DNA, 19 bp, synthetic construct
  source 1..19: mol_type = other DNA
gcaagcgtcatcttttacg

SEQ ID NO: 249 - DNA, 25 bp, synthetic construct
  source 1..25: mol_type = other DNA
gtccagtttttttttttttttccca

SEQ ID NO: 250 - DNA, 22 bp, synthetic construct
  source 1..22: mol_type = other DNA
cagtcaggtcttttcttgttca

SEQ ID NO: 251 - DNA, 24 bp, synthetic construct
  source 1..24: mol_type = other DNA
ccagtttttttttttttttcgggt

SEQ ID NO: 252 - DNA, 24 bp, synthetic construct
  source 1..24: mol_type = other DNA
cgcagtctggttaatctttatcag

SEQ ID NO: 253 - DNA, 25 bp, synthetic construct
  source 1..25: mol_type = other DNA
ggtccagtttttttttttttttgac

SEQ ID NO: 254 - RNA, 23 nt, synthetic construct
  source 1..23: mol_type = other RNA
gcgacttgtaacgtcagaggcgt

SEQ ID NO: 255 - RNA, 22 nt, synthetic construct
  source 1..22: mol_type = other RNA
caagcgtcatcttttacgtggg

SEQ ID NO: 256 - RNA, 23 nt, synthetic construct
  source 1..23: mol_type = other RNA
tcaggtcttttcttgttcacccg

SEQ ID NO: 257 - RNA, 22 nt, synthetic construct
  source 1..22: mol_type = other RNA
tctggttaatctttatcaggtc

SEQ ID NO: 258 - DNA, 20 bp, synthetic construct
  source 1..20: mol_type = other DNA
aatgattaacccgccatgct

SEQ ID NO: 259 - DNA, 18 bp, synthetic construct
  source 1..18: mol_type = other DNA
gggaggtgtgggaggttt

SEQ ID NO: 260 - DNA, 25 bp, synthetic construct
  source 1..25: mol_type = other DNA
acttatctacagatctgcggccgct

SEQ ID NO: 261 - DNA, 22 bp, synthetic construct
  source 1..22: mol_type = other DNA
ggcgtacttggcatatgataca

SEQ ID NO: 262 - DNA, 22 bp, synthetic construct
  source 1..22: mol_type = other DNA
agtaacgccaatagggactttc

SEQ ID NO: 263 - DNA, 24 bp, synthetic construct
  source 1..24: mol_type = other DNA
ttacggtaaactgcccacttggca